(19) United States
(12) Patent Application Publication
(10) Pub. No.: US 2022/0409716 A1
(43) Pub. Date: Dec. 29, 2022

(54) T-CELL COMPOSITIONS AND METHODS OF MAKING AND USING THE SAME

(71) Applicants: Children's National Medical Center, Washington, DC (US); The United States of America, as represented by the Secretary, Dept. of Health and Human Service, Bethesda, MD (US)

(72) Inventors: Michael Keller, Laurel, MD (US); Catherine Mary Bollard, Bethesda, MD (US); Ryo Hanajiri, Bethesda, MD (US); Gelina Sani, Washington, DC (US); Lisbeth Kim Green, Olney, MD (US); Jeff Cohen, Silver Spring, MD (US); Stanislav V. Sosnovtsev, North Potomac, MD (US)

(21) Appl. No.: 17/763,548

(22) PCT Filed: Sep. 24, 2020
(86) PCT No.: PCT/US20/52559

§ 371 Date: Mar. 24, 2022

Related U.S. Application Data
(60) Provisional application No. 62/904,856, filed on Sep. 24, 2019.

(51) Int. Cl. A61K 39/12; A61P 31/14

(52) U.S. Cl. CPC A61K 39/12; A61P 31/14; A61K 2039/572

(57) ABSTRACT

The present disclosure provides adoptive T-cell compositions, therapies, and processes of manufacture that are tailored for treatment or prevention of a subject with a norovirus infection, and, in some embodiments, for those subjects who are candidates for hematopoietic stem cell treatments, subjects who have undergone hematopoietic stem cell treatments, and subjects that have, are diagnosed with or suspected of having primary immunodeficiency disorders.

### CROSS-REFERENCE TO RELATED APPLICATIONS

This application claims priority to and benefit of U.S. Provisional Application No. 62/904,856, filed Sep. 24, 2019, entitled “T-Cell Compositions and Methods of Making and Using the Same,” the content of which is incorporated herein by reference in its entirety.

### GOVERNMENT SUPPORT

This disclosure was made with funding from the US government under US Grant No. K23-HL136783-01 provided from the National Institutes of Health under the Department of Health and Human Services. The United States government may have rights in this disclosure.

### FIELD OF THE DISCLOSURE

The present disclosure provides adoptive T-cell compositions, therapies, and processes of manufacture that are tailored for treatment or prevention of a subject with a norovirus infection, and, in some embodiments, for those subjects who are candidates for hematopoietic stem cell treatments, subjects who have undergone hematopoietic stem cell treatments, and subjects that have, are diagnosed with or suspected of having primary immunodeficiency disorders.

### BACKGROUND

Norovirus is a positive-sense, single-strand RNA virus of the family Caliciviridae, and is a leading worldwide cause of acute gastroenteritis [1,2]. The genome is organized into open reading frames (ORFs) 1, 2, and 3, which encode nonstructural proteins NS1-7, major capsid protein VP1, and minor capsid protein VP2, respectively. Human norovirus has high genetic diversity, and it clusters into three genogroups (GI, GII, and GIV) and over 25 genotypes. Of these, GII.4 is responsible for the majority of human infections and outbreaks. These infections in immunocompetent hosts typically cause self-limiting nausea, vomiting, and diarrhea lasting 1-3 days. However, norovirus infection can result in prolonged symptoms that persist for months to years, particularly in immunocompromised patients, leading to chronic diarrhea and malabsorption [3,4]. The exact prevalence of norovirus infection among immunocompromised hosts is unknown, but norovirus was identified in 13% of immunocompromised patients with diarrhea [5]. The severe impact of chronic norovirus infection has become increasingly evident among patients with primary immunodeficiencies [6,7], solid organ transplants [8,9], and hematopoietic stem cell transplants [10]. Currently, there are no approved antiviral therapies to treat norovirus and no approved vaccines for disease prevention.

While advances in human norovirus therapeutic and vaccine research have been hampered by the absence of animal models and the inability to cultivate norovirus in vitro, efforts to understand protective immunity against norovirus are ongoing. In the animal model of murine norovirus infections, both B-cells and T-cells are necessary to clear infection [11,12]. In human case reports and case series, the return of T-cell function after HIV treatment, stem cell transplantation or gene therapy was necessary to clear norovirus infection [13,14]. Norovirus-specific memory T-cell responses have recently been identified in healthy donors [15]. To date, however, only a single CD8+-restricted epitope in the major structural protein VP1 has been described and the breadth of T-cell responses to human norovirus remains unknown [16].

### SUMMARY

The present disclosure relates to adoptive immunotherapy, compositions comprising isolated and primed T cells to treat norovirus, and compositions that comprise ex vivo expansion of norovirus-specific T-cells (NSTs) that provide a new therapeutic strategy against acute or chronic norovirus infection. The disclosure relates to methods of generating NSTs from peripheral blood of healthy donors using a good manufacturing practice (GMP)-compliant methodology. The disclosure also relates to methods of stimulating an antigenic-specific cellular immune response and to methods of stimulating an immunodominant antigen-specific immune response against novel epitopes within norovirus antigens, including, but not limited to, NS6 and VP1 antigens. The present disclosure relates to a cell comprising a T-cell receptor specific for norovirus antigens, including but not limited to NS6 and VP1 antigens or functional fragments thereof.

Provided herein are compositions and methods for use T-cell therapy to treat acute or chronic norovirus infection. Non-engineered T-cell compositions that include in the same dosage a multiplicity of T-cell subpopulations are provided for administration to a human patient with acute or chronic norovirus infection, wherein each T-cell subpopulation is primed with one or more norovirus antigens disclosed herein, and the T-cell subpopulations comprised in the T-cell composition for administration are chosen specifically based on the norovirus immunodominant antigens detected in a subject infected with norovirus. By using separate activated T-cell subpopulations to form the T-cell composition for administration, the T-cell composition as a whole includes individual T-cell subpopulations targeting norovirus-specific antigens (NSAs), resulting in a highly consistent and activated T-cell composition capable of targeting cells infected with norovirus, chronically or acutely.

The disclosure also relates to a composition comprising T-cell subpopulations that comprise one or a plurality of T-cell receptors capable of binding and/or targeting one or a plurality of norovirus antigens. The generation of each T-cell subpopulation can be accomplished through the ex vivo priming and activation of the T-cell subpopulation with one or more peptides from a norovirus, such as NS6 and VP1 antigens from norovirus or functional fragments thereof. In certain embodiments, if more than one peptide from a single, targeted norovirus antigen is used for priming, the peptide segments can be generated synthetically or recombinantly by making overlapping peptide fragments of the norovirus antigen, as provided for example in commercially available overlapping peptide libraries or “PepMix™.” In particular embodiments, the disclosure relates to a T cell population comprising at least one or a plurality of T cell receptors targeting peptides of the overlapping peptide library are 7, 8, 9, 10, 11, 12, 13, 14, 15, 16, 17, 18, 19, 20, 21, 22, 23, 24, 25, 26, 27, 28, 29, 30, 31, 32, 33, 34, or 35 or more amino acids in length, for example, and there is overlap of 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length. In certain embodiments, if more than one peptide from a single, targeted norovirus antigen is used for priming the cells, the peptide segments can be generated by making overlapping peptide fragments of the norovirus antigen, as provided for example in commercially available overlapping peptide fragments, and further enriched with certain antigenic epitopes of the targeted norovirus that are active through specific cell donor human leukocyte antigen (HLA) alleles, for example, a single specific HLA-restricted epitope or multiple specific HLA-restricted epitopes of the norovirus. In certain embodiments, if more than one peptide from a single, targeted norovirus antigen or a plurality of norovirus antigens are used, the peptide segments can be selected from certain antigenic epitopes of the targeted norovirus antigen that are active through specific HLA alleles, for example, a single specific HLA-restricted epitope or multiple specific HLA-restricted epitopes of norovirus.

In some embodiments, the disclosure relates to a composition comprising a T-cell subpopulation primed with a single norovirus peptide mix, wherein the peptide mix comprises antigenic epitopes derived from a norovirus antigen based on one or more of the donor's HLA phenotypes, for example, the peptides are restricted through one or more of the cell donor's HLA alleles such as, but not limited to, HLA-A, HLA-B, and HLA-DR. By including specifically selected donor HLA-restricted peptides from norovirus in the peptide mix for priming and expanding each T-cell subpopulation, a T-cell subpopulation can be generated that provides greater norovirus targeted activity through one or more donor HLA alleles, improving potential efficacy of the T-cell subpopulation for patients that share at least one HLA allele with the donor. In addition, by generating a T-cell subpopulation with norovirus-specific antigen targeted activity through more than one donor HLA allele, a single donor T-cell subpopulation may be included in a disclosed composition for multiple recipients with different HLA profiles by matching one or more donor HLA alleles showing norovirus activity. In some embodiments, the norovirus peptides used to prime and expand a T-cell subpopulation are generated based on a cell donor's HLA profile, wherein the peptides are HLA-restricted epitopes specific to at least one or more of a donor's HLA-A alleles, HLA-B alleles, or HLA-DR alleles, or a combination thereof. In some embodiments, the HLA-A alleles are selected from a group comprising HLA-A*01, HLA-A*02:01, HLA-A*03, HLA-A*11:01, HLA-A*24:02, HLA-A*26, and HLA-A*68:01. In some embodiments, the HLA-B alleles are selected from a group comprising HLA-B*07:02, HLA-B*08, HLA-B*15:01 (B62), HLA-B*18, HLA-B*27:05, HLA-B*35:01, and HLA-B*58:02. In some embodiments, the HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, and HLA-DRB1*1501 (DR2b). In some embodiments, the master mix of peptides includes both an overlapping peptide library and specifically selected HLA-restricted peptides generated by determining the HLA profile of the donor source.

In some embodiments, the composition comprises one or a plurality of T cells comprising T cell receptors (TCR) capable of binding a cell expressing a norovirus antigen. In some embodiments, the one or a plurality of T cells comprise a subpopulation of T cells that express a TCR capable of binding a cell expressing a norovirus antigen. In some embodiments, the norovirus antigen is a mutated form of a norovirus antigen derived through a single point mutation, a deletion, an insertion, or a frameshift mutation.

In some embodiments, the disclosed composition includes one or more T-cell subpopulations targeting mutant norovirus sequences. In some embodiments, the disclosed composition consists of individual T-cell subpopulations targeting NS1-2, NS3, NS4, NS5, NS6, NS7, VP1 and VP2, wherein the peptides used to generate the T-cell subpopulations are overlapping peptide libraries. In some embodiments, the disclosed composition consists of individual T-cell subpopulations targeting NS1, NS2, NS3, NS4, NS5, NS6, NS7, VP1 and VP2, wherein the peptides used to generate the T-cell subpopulations are functional fragments comprising at least from about 5 to about 20 amino acids of any about 5 to about 20 amino acid sequence from of any one or combination of the aforementioned targeted peptides. In some embodiments, the disclosed composition consists of individual T-cell subpopulations targeting NS1, NS2, NS3, NS4, NS5, NS6, NS7, VP1 and VP2, wherein the peptides used to generate the T-cell subpopulations are overlapping peptide libraries that have been further enriched with one or more specific known or identified epitopes expressed by a subject's virus-infected cell. In some embodiments, the disclosed composition consists of individual T-cell subpopulations targeting NS1, NS2, NS3, NS4, NS5, NS6, NS7, VP1 and VP2, wherein the peptides used to generate the T-cell subpopulations are specifically selected epitopes of the norovirus antigens that are HLA-restricted based on a cell donor's HLA type. In some embodiments, the HLA-restricted epitopes are specific to at least one or more of a cell donor's HLA-A alleles, HLA-B alleles, or HLA-DR alleles. In some embodiments, the HLA-A alleles are selected from a group comprising HLA-A*01, HLA-A*02:01, HLA-A*03, HLA-A*11:01, HLA-A*24:02, HLA-A*26, or HLA-A*68:01. In some embodiments, the HLA-B alleles are selected from a group comprising HLA-B*07:02, HLA-B*08, HLA-B*15:01 (B62), HLA-B*18, HLA-B*27:05, HLA-B*35:01, or HLA-B*58:02. In some embodiments, the HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, or HLA-DRB1*1501 (DR2b).

In some embodiments, a sample of the patient's virus-infected cells are taken by biopsy, or other isolation, and a profile of associated antigenic proteins expressed in the cells is identified and quantified, and the T-cell subpopulations of the disclosed composition target one or more of the expressed virus antigens. In another embodiment, an epitope profile of expressed antigenic proteins corresponding to the patient's infecting norovirus strain is identified, and the T-cell subpopulations of the disclosed composition target one or more of the identified epitopes.

In some embodiments, the T-cell subpopulations of the disclosed compositions are derived from an allogeneic donor, for example, from the peripheral blood, apheresis product, or bone marrow from a healthy donor. In some embodiments, the T-cell subpopulations for inclusion in the disclosed composition are from an allogeneic donor subject, for example, from the peripheral blood, apheresis product, or bone marrow from a healthy subject donor. In some embodiments, the T-cell subpopulations for inclusion in the disclosed composition are from umbilical cord blood.

In one aspect, the disclosure further includes a library of individual T-cell subpopulations, and methods of manufacturing a bank of individual T-cell subpopulations with an associated phenotypic characteristic database. The library includes individual T-cell subpopulations which have been primed and activated to a specific, single norovirus genotype. In some embodiments, the T-cell subpopulations are derived from an allogeneic donor source, for example, the peripheral blood, apheresis product or bone marrow from a healthy donor and/or umbilical cord blood sample. In some embodiments, the T-cell subpopulations are HLA-typed and/or the donor source recorded. The T-cell subpopulations' antigenic recognition response is verified and characterized, for example, via ELISPOT IFN-γ assay, IL-2 assay, TNF-α assay, or multimer assay to quantify the activity of the T-cell population against the specific, targeted norovirus antigen. Alternatively, the diversity of T-cell receptor (TCR) α-chain and β-chain repertoire can be characterized, for example, using TCR ligation-anchored-magnetically captured PCR (TCR-LA-MC PCR) (see, e.g., Ruggiero et al., High-resolution analysis of the human T-cell receptor repertoire, Nat. Comm. 2015 6:8081) or other appropriate characterization techniques. Furthermore, the T-cell subpopulations' antigenic recognition response is further characterized through its corresponding HLA-allele, for example through an HLA restriction assay. The T-cell subpopulations can be cryopreserved and stored. In some embodiments, the T-cell subpopulations are stored based on the donor source. In some embodiments, the T-cell subpopulations are stored by norovirus antigen specificity. In some embodiments, the T-cell subpopulations are stored by HLA subtype and restrictions. In some embodiments, the compositions of the disclosure are stored at −80 or −20 degrees Celsius or frozen in liquid nitrogen.

By characterizing each T-cell subpopulations' reactivity and corresponding HLA-allele, a disclosed composition can be optimized for each patient based on specific T-cell subpopulation reactivity and HLA matching, providing a highly personalized T-cell therapy. Accordingly, if a patient has a norovirus infection that expresses one epitope but not another, or if one norovirus epitope invokes a greater T-cell response, that T-cell subpopulation can be taken from the bank, thawed and used as the subpopulation with therapeutically effective effects in the disclosed composition. In this way, the T-cell therapy can be tailored to evoke a maximal response against the patient's virus infection. In some embodiments, the disclosure therefore relates to a method of priming a T cell population or manufacturing an antigene-specific T cell population against one or a plurality of norovirus antigens from a subject comprising a norovirus infection, the method comprising: (i) identifying a subject's HLA-allele corresponding to immunoreactivity; (ii) identifying at least one or a plurality of norovirus epitopes from the subject corresponding to immunoreactivity; (iii) creating a peptide library specific for the subject comprising one or a plurality of the epitopes; (iv) exposing the peptide library to isolated T cells from the subject or a donor subject for a time period sufficient to stimulate propagation of an epitope-specific T cell subpopulation specific for the one or plurality of norovirus epitopes; and (v) isolating the epitope-specific T cells; and (vi) administering a therapeutically effective amount of the epitope-specific T cells to the subject infected with norovirus. In some embodiments, the immunoreactivity of the HLA allele and the norovirus epitopes are determined by isolating a sample comprising one or a plurality of lymphocytes from a subject and exposing them to one or a plurality of norovirus antigens and/or one or a plurality of antibodies specific for one or a plurality of T cell receptors and/or HLA molecules in vitro. In some embodiments, the immunoreactivity is determined by ELISA and/or flow cytometry.

This disclosure thus acknowledges and accounts for the fact that T-cells from various donors may have variable activity against the same norovirus antigen, or even the same epitope, generating T-cell responses with varying efficiency. This fact is taken into account when producing the comprehensive bank of a wide variety of allogeneic activated T-cells for personalized T-cell therapeutic composition of the disclosure. Derived T-cell subpopulations having shared HLA-alleles that exhibit strong activity to an epitope or virus antigen expressed in virus-infected cells can be selected from the bank for inclusion in the disclosed composition. In some embodiments, one or more of the T-cell subpopulations for consideration for inclusion in the disclosed composition are tested against cells expressing the norovirus antigens prior to administration in vivo by exposing cells infected with the patient's virus to the one or more T-cell subpopulations and determining the T-cell subpopulation's ability to lyse the virally infected cells. In this way, the probability of the disclosed composition inducing a therapeutic response upon administration to the patient is greatly enhanced.

In some embodiments, provided herein is a method of treating a subject with primary immunodeficiency disease and/or norovirus infection comprising:

i) determining the HLA subtype of the subject;

ii) identifying one or a plurality of norovirus antigens associated with the virus type for targeting with norovirus-specific T-cell subpopulations;

iii) selecting one or a plurality of stored T-cell subpopulation having the highest activity against each targeted norovirus antigen through one or more HLA-alleles shared between the subject and the T-cell subpopulations, wherein each T-cell subpopulation is specific for a norovirus antigen, wherein each of the T-cell subpopulations is specific for a different norovirus antigen, wherein each of the T-cell subpopulations is primed and expanded separately from each other, wherein each of the T-cell subpopulations is primed and expanded ex vivo;

iv) combining each selected stored T-cell subpopulation to create a disclosed composition; and

v) administering a therapeutically effective amount of the disclosed composition to the subject.

In some embodiments, provided herein is a method of treating a subject with primary immunodeficiency disease and/or norovirus infection comprising:

i) determining the HLA subtype of the subject;

ii) diagnosing the virus infection of the subject;

iii) identifying one or a plurality of norovirus antigens associated with the virus type for targeting with norovirus-specific T-cell subpopulations;

iv) selecting one or a plurality of stored T-cell subpopulation having the highest activity against each targeted norovirus antigen through one or more HLA-alleles shared between the subject and the T-cell subpopulations, wherein each T-cell subpopulation is specific for a norovirus antigen, wherein each of the T-cell subpopulations is specific for a different norovirus antigen, wherein each of the T-cell subpopulations is primed and expanded separately from each other, wherein each of the T-cell subpopulations is primed and expanded ex vivo;
v) combining each selected stored T-cell subpopulation to create a disclosed composition; and
vi) administering a therapeutically effective amount of the disclosed composition to the patient.

In some embodiments, provided herein is a method of treating a patient with an acute or chronic norovirus infection comprising:
i) determining the HLA subtype of the patient;
ii) determining the viral expression profile of the patient's tissue;
iii) identifying one or a plurality of norovirus antigens expressed by the patient's tissue for targeting with norovirus-specific T-cell subpopulations;
iv) selecting one stored T-cell subpopulation having the highest activity against each targeted norovirus antigen through one or more HLA-alleles shared between the patient and the T-cell subpopulations, wherein each T-cell subpopulation is specific for a single norovirus antigen, wherein each of the T-cell subpopulations is specific for a different norovirus antigen, wherein each of the T-cell subpopulations is primed and expanded separately from each other, wherein each of the T-cell subpopulations is primed and expanded ex vivo;
v) combining each selected stored T-cell subpopulation to create a disclosed composition; and,
vi) administering a therapeutically effective amount of the disclosed composition to the patient.

In some embodiments, the shared HLA alleles are selected from one or more of HLA-A, HLA-B, or HLA-DR.

The disclosure relates to a method of treating a subject in need thereof comprising administering to the subject in need thereof a composition comprising a therapeutically effective amount of a T-cell population comprising a T cell receptor capable of binding a cell of the subject infected with norovirus. In some embodiments, the T cell population is stimulated with one or combination of norovirus antigens chosen from: NS1-2, NS3, NS4, NS5, NS6, NS7, VP1, and VP2, or functional fragments thereof that comprise at least about 70%, 75%, 80%, 85%, 90%, 95%, 96%, 97%, 98%, or 99% sequence identity to NS1-2, NS3, NS4, NS5, NS6, NS7, VP1, or VP2, and wherein the T-cell population comprising a T cell receptor capable of binding a cell of the subject. In some embodiments, the T cell population expresses at least one HLA allele of the subject in need thereof.

In some embodiments, the T-cell subpopulations used to create the disclosed composition are combined in about an equal ratio. In some embodiments, the T-cell subpopulations used to create the disclosed composition are combined in a variable ratio. In some embodiments, the disclosed composition comprises a first T-cell subpopulation and a second T-cell subpopulation, wherein the first T-cell subpopulation is specific for a different norovirus antigen than the second T-cell subpopulation. In some embodiments, the ratio of the first and second T-cell subpopulations is fixed at an equal ratio of about 1:1, wherein the ratio is based on either total cell number or normalized cell activity. In alternative embodiments, the separate T-cell subpopulations are not combined into a single dosage form, but rather administered as separate compositions, wherein the separate compositions are administered concomitantly in a ratio described above.

The ratios of the T-cell subpopulations in the disclosed composition may be selected based on the knowledge of the patient's infecting norovirus characteristics or the health provider's best judgement. In some embodiments, the composition comprises (i) at least about 45%, 50%, 55%, 60%, 65%, 70%, 75%, 80%, or 85% of a first T-cell subpopulation, and (ii) at least about 15%, 20%, 25%, 30%, 35%, 40%, 45%, 50%, or 55% of a second T-cell subpopulation, wherein the percentage adds to 100% by either total cell number or normalized cell activity. In some embodiments, the ratio or percentage of each T-cell subpopulation is normalized based on the measured activity of each T-cell subpopulation against the norovirus antigen as measured by, for example, but not limited to, the ELISpot assay. In some embodiments, the percentage of the first and second T-cell subpopulations is based on the antigen expression profile of a norovirus-infected cell such that the percentage of the first and second T-cell subpopulations correlates with the norovirus antigen expression profile.

In an alternative embodiment, the separate T-cell subpopulations are not combined into a single dosage form, but rather administered as separate compositions, wherein the separate compositions are administered concomitantly.

By monitoring the levels of circulating antigen-specific T-cells and the norovirus expression profile of the patient's virus following administration of the first disclosed composition, the T-cell subpopulations included in any second, third, or subsequently administered disclosed composition can be adjusted, providing a more tailored approach to treatment as norovirus infection progresses. For example, if after an initial administration of a disclosed composition containing for example a T-cell subpopulation to NS6, if high levels of circulating NS6-specific T-cells are measured, then it may not be necessary to include a NS6-specific T-cell subpopulation in the subsequently administered disclosed compositions. Similarly, if after an initial administration of a disclosed composition containing for example a NS6 T-cell subpopulation, mutations in NS6 epitopes are measured in the patient's infecting viral strain that are not recognized by the infused NSTs, then it may not be necessary to include the NS6-specific T-cell subpopulation in subsequent disclosed compositions. Accordingly, the subsequently administered disclosed compositions may be modified to more closely reflect the norovirus antigen expression profile of the patient's infecting norovirus. In addition, the subsequently administered disclosed compositions may be modified based on the ongoing T-cell subpopulation responses in vivo, whereby previously administered T-cell subpopulations showing robust activity in vivo are not included in subsequent disclosed compositions because additional administrations of that specific T-cell subpopulation may be unnecessary. In some embodiments, the first, second, and any subsequent disclosed compositions are comprised of T-cell subpopulations derived from the same donor. In alternative embodiments, the first, second, and subsequent disclosed compositions may be derived from different donors, provided that one of the donors is a non-umbilical cord blood donor.

In some embodiments, the present disclosure includes a method for generating a T-cell subpopulation specific to a norovirus antigen comprising:
i) identifying eligible donors who are negative to the patient's disease, and preferably healthy, and wherein the donor sample can be cord blood or peripheral blood mononuclear cells (PBMCs);
ii) collecting the mononuclear cells from the healthy donor and optionally removing any effector or other memory T-cells optionally based on CD45RA⁻, CD45RO⁺, CCR7⁻, CD62L⁻, CCR7⁺, and/or CD62L⁺ markers;
iii) separating the mononuclear cells into two components;
iv) separating the cells in the first component into non-adherent T-cells and precursors and adherent dendritic cells and precursors, using any method known in the art, for example exposure to a solid medium, separation magnetically, use of antibodies, etc., and if not done already, optionally removing any effector or other memory T-cells optionally based on CD45RA⁻, CD45RO⁺, CCR7⁻, CD62L⁻, CCR7⁺, and/or CD62L⁺ markers;
v) differentiating monocytes and precursors to dendritic cells with IL-4 and GM-C SF, followed by treatment with maturing cytokines such as LPS, TNFα, IL-1β, IL-4, IL-6 and GM-CSF, and then pulsing with one or more peptide(s) and/or epitope(s) from a single selected norovirus antigen; and then irradiating to form dendritic antigen presenting cells (APCs);
vi) treating the non-adherent T-cells and precursors with cytokines IL-7 and IL-15 to polarize (e.g. differentiate) to Th1 cells (and in some embodiments, without the use of IL-12);
vii) mixing the dendritic antigen presenting cells from (v) with the non-adherent T-cells and T-cell precursor cells from (vi) in the presence of cytokines IL-6, optionally in a ratio of between about 5:1 and about 20:1 of (vi) to (v) to produce a T-cell subpopulation specific for the single selected norovirus antigen;
viii) treating the second component of mononuclear cells with a mitogen such as phytohemagglutinin (PHA), T-blast, B-blast, lymphoblastic cell, or CD3/CD28 Blast optionally in the presence of IL-2 to produce activated T-cells; and then irradiating the cells to inhibit growth;
ix) pulsing the PHA blasts in (viii) with selected antigenic peptide(s) and/or epitope(s) from the single selected norovirus antigen and irradiating to inhibit growth;
x) mixing the antigen specific T-cells from (vii) with the activated T-cell subpopulation from (ix) optionally in the presence of K562 accessory cells (preferably HLA-negative, K562 cells expressing CD80, CD83, CD86 and/or 4-IBBL) or LPS, and optionally IL-15 and/or IL-2;
xi) recovering the produced single epitope-specific T-cell subpopulation;
xii) optionally characterizing the resulting T-cell subpopulation for banking; and
xiii) optionally cryopreserving and storing in the bank until use.

In the above process, unless specific steps are taken to remove cell components of the donor blood starting material, for example, removal based on cell surface markers, etc., the final T-cell subpopulation comprise one or a plurality of cells chosen from: Natural Killer T-cells, γδ T-cells, CD4+ T-cells, and CD8+ (cytotoxic) T-cells, among others, and may have naïve, and effector memory or central memory cells. The ratios of these cell types in the disclosed composition will vary according to the donor's blood and processing conditions.

In another aspect, the present disclosure includes a method of manufacturing a T-cell subpopulation of the present disclosure comprising
i) collecting a mononuclear cell product from a healthy donor;
ii) determining the HLA subtype of the mononuclear cell product;
iii) separating the monocytes and the lymphocytes of the mononuclear cell product;
iv) generating and maturing dendritic cells (DCs) from the monocyte fraction;
v) pulsing the DCs with one or more peptides and/or epitopes from a single norovirus antigen or functional fragment thereof;
vi) carrying out a CD45RA+ selection to isolate naïve lymphocytes from the lymphocyte fraction;
vii) stimulating the naïve lymphocytes with the peptide-pulsed DCs in the presence of a cytokine cocktail;
viii) repeating the T-cell stimulation with fresh peptide-pulsed DCs or other peptide-pulsed antigen presenting cells in the presence of a cytokine cocktail;
ix) harvesting the T-cell subpopulation;
x) characterizing the T-cell subpopulation as described herein; and
xi) storing or banking the T-cell subpopulation for future use in a disclosed composition.

In some embodiments, the disclosed norovirus peptides or functional fragments thereof used to prime and expand a T-cell subpopulation in step (v) are from a library of overlapping peptide fragments of the norovirus antigen, which can be customly made commercially (e.g., PepMix™). In some embodiments, the norovirus peptides used to prime and expand a T-cell subpopulation in step (v) include specifically selected HLA-restricted peptides generated by determining the HLA profile of the donor source, and including peptide epitopes derived from the targeted norovirus antigen that are active through the donor's HLA type. In other embodiments, the present disclosure includes a method of manufacturing a T-cell subpopulation of the present disclosure comprising:
i) collecting a mononuclear cell product from a healthy donor;
ii) determining the HLA subtype of the mononuclear cell product;
iii) separating the monocytes and the lymphocytes of the mononuclear cell product;
iv) generating and maturing dendritic cells (DCs) from the monocyte fraction;
v) pulsing the DCs with one or more peptides and/or epitopes from a single norovirus antigen or functional fragment thereof;
vi) carrying out a CD45RA+ selection to isolate naïve T-cells from the lymphocyte fraction;
vii) stimulating the naïve T-cells with the peptide-pulsed DCs in the presence of a cytokine cocktail;
viii) repeating the T-cell stimulation with fresh peptide-pulsed DCs or other peptide-pulsed antigen presenting cells in the presence of a cytokine cocktail creating a primed T-cell subpopulation;
ix) harvesting the primed T-cell subpopulation;
x) characterizing the primed T-cell subpopulation as described herein; and
xi) banking the primed T-cell subpopulation for future use in a disclosed composition.

In a further aspect, the present disclosure includes a library of isolated T-cell subpopulations targeting a norovirus comprising two or more characterized T-cell subpopulations. The T-cell subpopulations are characterized, the characterization is recorded in a database for future use, and the T-cell subpopulations are cryopreserved. The T-cell subpopulation has been characterized by, for example, HLA-phenotype, its specificity to its specific norovirus antigen, the epitope or epitopes each T-cell subpopulation is specific to, which MHC Class I and Class II the T-cell subpopulation is restricted to, antigenic activity through the T-cell's corresponding HLA-allele, and immune effector subtype concentration. In some embodiments, the steps of any of the methods comprising isolating or selecting cell populations are accomplished by flow cytometry specific for one or a plurality of T cell specific biomarkers on the surface of the cell. In some embodiments, the biomarker is presence of detectable levels of TCR, CD45RA, HLA alleles, CD4, CD8 and/or functional fragments thereof

### BRIEF DESCRIPTION OF THE DRAWINGS

FIG. 1A-1D show that norovirus-specific T-cells can be generated from peripheral blood of norovirus-seropositive donors. FIG. 1A: Peripheral blood mononuclear cells from healthy donors were stimulated with norovirus pepmixes on day 0. Fold expansion was measured 10 days after stimulation based on absolute cell counting in total cell numbers (n=20, mean±standard error of the mean [SEM]). FIG. 1B: Phenotype of the expanded cells accessed by flow cytometry for T-cell markers (CD3, CD4, and CD8), natural killer (NK) cell markers (CD16), and memory subsets (CD45RO and CD62L) (n=9, mean±SEM). FIG. 1C: Specificity of the expanded cells with response to norovirus antigens stimulation was assayed by interferon (IFN)-γ enzyme-linked immunospot (ELISpot) assay (n=20, mean±SEM). Unstimulated T cells (CTL only) and stimulation with actin were used as negative controls. Results are presented as spot-forming cells (SFC)/1×105 cells. The number of spots were compared with those from actin control (*, P<0.05 and **, P<0.01; two-tailed Student's t-test). FIG. 1D: Specificity of the expanded cells against norovirus antigens between norovirus seropositive donors (n=17) and seronegative donors (n=3) by IFN-γ ELISpot (mean±SEM). CM, central memory T-cells (CD45RO⁺CD62L⁺); EM, effector memory T-cells (CD45RO⁺CD62L⁻); NKs, natural killer cells (CD3⁻CD16⁺); NKTs, natural killer T cells (CD3⁺CD16⁺); T_{reg}, regulatory T cells (CD3⁺CD4⁺CD25⁺CD127^{dim}).

FIG. 2A-2E show that norovirus-specific T-cells are polyfunctional. Norovirus-specific T-cells were tested for secretion of pro-inflammatory cytokines (FIG. 2A) and regulatory cytokines (FIG. 2B) in response to stimulation with pooled pepmixes. Cytokines were measured in cell supernatant after 24-hour stimulation by Luminex assay. Stimulation with actin was used as a control (n=7, mean±standard error of the mean [SEM]). FIG. 2C: Polycytokine (interferon [IFN]-γ and tumor necrosis factor [TNF]-α) production in response to pooled norovirus pepmixes stimulation as evaluated by intracellular cytokine staining from CD4⁺ and CD8⁺ T-cells in one representative donor (gated on CD3⁺). Stimulation with actin was used as negative control. FIG. 2D: Summary results of double cytokine (IFN-γ and TNF-α) production cells in CD4⁺ and CD8⁺ T-cell populations (n=6, mean±SEM). FIG. 2E: Expression of co-inhibitory markers of norovirus-specific T-cells by CD4⁺ and CD8⁺ T cells after restimulation with pooled norovirus pepmixes (n=9, mean±SEM). GM-CSF, granulocyte-macrophage colony-stimulating factor; IL, interleukin.

FIG. 3A-3F show identification of VP1 CD8-restricted T-cell epitopes. T-cell epitope mapping for VP1 protein was performed and results from one representative donor is shown. FIG. 3A: The breadth of T-cell reactivity was evaluated using a total of 21 mini-peptide pools, each containing 8-12 peptides, spanning the entire VP1 protein. Mini-pools were made in such a way that each peptide was present in two pools. A norovirus-specific T-cell product was stimulated with 21 mini-peptide pools and the T-cell responses were measured by interferon (IFN)-γ enzyme-linked immunospot (ELISpot). Responses to pools 3 and 10, 3 and 15, 3 and 16, 3 and 19, 3 and 21, 4 and 10, 4 and 15, 4 and 16, 4 and 19, 4 and 21, 7 and 10, 7 and 15, 7 and 16, 7 and 19, and 7 and 21 may be induced by individual 15-mer peptides 363, 368, 369, 372, 374, 375, 380, 381, 384, 386, 411, 416, 417, 420, and 422, respectively (cells in gray). FIG. 3B: Testing of these single peptides by IFN-γ ELISpot revealed recognition of the single 15-mer peptides of 374, 375, 380, 381, 386, and 420 (mean±standard error of the mean [SEM]). FIG. 3C: Intracellular IFN-γ staining was used to evaluate the human leukocyte antigen (HLA)-restriction of the single 15-mer peptides (gated on CD3⁺). Results indicating CD8-restricted epitopes are shown. FIG. 3D: Blocking experiments with HLA class I and II blocking antibodies by IFN-γ ELISpot (mean±SEM). FIG. 3E: To identify the minimal 9-mer epitope, recognition against a panel of 9-mers overlapping by 8 amino acids spanning the sequence of 420 was evaluated by IFN-γ ELISpot (mean±SEM). FIG. 3F: Restricted HLA allele was evaluated by IFN-γ ELISpot using allogeneic HLA-partially matched PHA blasts alone or pulsed with GRNTHNVHL (GRN) peptide (mean±SEM). Autologous PHA blasts pulsed with GRN were used as a positive control. SFC, spot-forming cells.

FIG. 4A-4E show cross reactivity of norovirus-specific T-cells to various epitopes. Norovirus-specific T cells were stimulated with CD8-restricted T-cell epitopes GRNTHNVHL (FIG. 4A), YVVIGVHTA (FIG. 4B), FPGEQLLFF (FIG. 4C), and CD4-restricted T-cell epitope PIKQIQIHKSGEFCR (FIG. 4D), as well as altered versions as identified in database or clinical isolates. Responses were measured in IFN-γ ELISpot (mean±SEM). FIG. 4E: Cross-reactivity of norovirus-specific T-cells against mutated epitopes were measured by IFN-γ ELISpot assay. Data are cross-reactivity indices (variant response/GII.4 Sydney2012 response) of 45 NS6 (n=9) and 26 VP1 epitopes (n=11) (mean±SEM). SFC, spot-forming cells.

FIG. 5A-5B show that norovirus-specific T-cells can be generated from peripheral blood. FIG. 5A: PBMCs from healthy donors were stimulated with norovirus NS6 and VP1 pepmixes on day 0. Fold expansion measured 10 days after stimulation based on absolute cell counting (n=15, mean±SEM). FIG. 5B: Specificity of the expanded cells with response to norovirus NS6/VP1 antigens stimulation by IFN-γ ELISpot assay (n=15, mean±SEM). Unstimulated T-cells (CTL only) and stimulation with actin was used as a negative control. Results are presented as SFC/1×10⁵ cells.

FIG. 6A-6F show identification of NS6 CD8+-restricted T-cell epitopes. T-cell epitope mapping for NS6 protein was performed and results from one representative donor is shown. FIG. 6A: The breadth of T-cell reactivity was evaluated using a total of 4 mini-peptide pools, each containing 11-12 peptides, spanning the entire NS6 protein. A norovirus-specific T-cell product was stimulated with 4 mini-peptide pools and the T-cell responses were measured by IFN-γ ELISpot. Responses to pools 2 and 4 may be induced by individual 15mer peptides 211-219, and 229-236, respectively (cells in gray). FIG. 6B: Testing of these single peptides by IFN-γ ELISpot revealed recognition of the single 15-mer peptides of 211, 214, 215, 232, and 233 (mean±SEM). FIG. 6C: Intracellular IFN-γ staining was used to evaluate the HLA-restriction of the single 15-mer peptides (gated on CD3+). Results indicating CD8+-restricted epitopes are shown. Both CD8+ and CD8− subsets of the T-cells released INF-γ with responses to 15-mer peptide 232 GNDYVVIGVHTAAAR (GND), indicating that this peptide contained HLA class I and II-restricted epitopes. FIG. 6D: Blocking experiments with HLA class I and II blocking antibodies by IFN-γ ELISpot (mean±SEM). FIG. 6E: To identify the minimal 9-mer epitope, recognition against a panel of 9-mers overlapping by 8 amino acids spanning the sequence of 232 GND was evaluated by IFN-γ ELISpot (mean±SEM). FIG. 6F: Restricted HLA allele was evaluated by IFN-γ ELISpot using allogeneic HLA-partially matched PHA blasts alone or pulsed with YVV peptide (mean±SEM). Autologous PHA blasts pulsed with YVV were used as a positive control.

### DETAILED DESCRIPTION OF THE DISCLOSURE

Provided herein are improved adoptive T-cell therapies to treat human norovirus infections which include administering to a subject in need thereof a therapeutically effective amount of a T-cell composition that includes in the same dosage form a multiplicity of T-cell subpopulations, wherein each T-cell subpopulation is specific for one or a plurality of norovirus antigens. In some embodiments, the T-cell subpopulations comprised in the T-cell composition for administration are chosen specifically based on the norovirus antigen profile of the virus found in the subject.

Further, importantly, this advantageous T-cell therapy can be optimized for personal efficacy in the subject by maximizing cross-reactivity of included viral epitopes, thus improving the breadth of clinical viral strains targeted by the T-cells.

### Definitions

Unless defined otherwise, all technical and scientific terms used herein have the same meaning as commonly understood by one of ordinary skill in the art to which the disclosure pertains.

The term “a” and “an” refers to one or to more than one (i.e., to at least one) of the grammatical object of the article. By way of example, “an element” means one element or more than one element.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value as such variations are appropriate to perform the disclosed methods. When “about” is present before a series of numbers or a range, it is understood that “about” can modify each of the numbers in the series or range.

The term “at least” prior to a number or series of numbers (e.g. “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range.

The term “allogeneic” as used herein refers to medical therapy in which the donor and recipient are different individuals of the same species.

The term “antigen” as used herein refers to molecules, such as polypeptides, peptides, or glyco- or lipo-peptides that are recognized by the immune system, such as by the cellular or humoral arms of the human immune system. The term “antigen” includes antigenic determinants, such as peptides with lengths of 6, 7, 8, 9, 10, 11, 12, 13, 14, 15, 16, 17, 18, 19, 20, 21, 22 or more amino acid residues that bind to major histocompatibility complex (MHC) molecules, form parts of MHC Class I or II complexes, or that are recognized when complexed with such molecules.

The term “antigen presenting cell (APC)” as used herein refers to a class of cells capable of presenting one or more antigens in the form of peptide-MHC complex recognizable by specific effector cells of the immune system, and thereby inducing an effective cellular immune response against the antigen or antigens being presented. Examples of professional APCs are dendritic cells and macrophages, though any cell expressing MHC Class I or II molecules can potentially present peptide antigen.

The term “autologous” as used herein refers to medical therapy in which the donor and recipient are the same person.

The term “cord blood” as used herein has its normal meaning in the art and refers to blood that remains in the placenta and umbilical cord after birth and contains hematopoietic stem cells. Cord blood may be fresh, cryopreserved, or obtained from a cord blood bank.

The term “cytokine” as used herein has its normal meaning in the art. Nonlimiting examples of cytokines used in the disclosure include interleukin-2 (IL-2), IL-6, IL-7, IL-12, IL-15, and IL-27.

The term “cytotoxic T-cell” or “cytotoxic T lymphocyte” as used herein is a type of immune cell that bears a CD8+ antigen and that can kill certain cells, including foreign cells, tumor cells, and cells infected with a virus. Cytotoxic T-cells can be separated from other blood cells, grown ex vivo, and then given to a patient to kill tumor or viral cells. A cytotoxic T-cell is a type of white blood cell and a type of lymphocyte.

The term “dendritic cell” or “DC” as used herein describes a diverse population of morphologically similar cell types found in a variety of lymphoid and non-lymphoid tissues, see Steinman, Ann. Rev. Immunol. 9:271-296 (1991).

The term “effector cell” as used herein describes a cell that can bind to or otherwise recognize an antigen and mediate an immune response. Tumor-, virus-, or other antigen-specific T-cells and NKT-cells are examples of effector cells.

The term “endogenous” as used herein refers to any material from or produced inside an organism, cell, tissue or system.

The term “epitope” or “antigenic determinant” as used herein refers to the part of an antigen that is recognized by the immune system, specifically by antibodies, B cells, or T cells.

The term “exogenous” as used herein refers to any material introduced from or produced outside an organism, cell, tissue or system.

The term “functional fragment” as used herein refers to a portion of a full-length of the norovirus antigen, such as NS1, NS2, NS3, NS4, NS5, NS6, NS7, VP1 and VP2, that retains some or all of the activity (e.g., biological activity) of the full-length polypeptide. The functional fragment can be any size, provided that the fragment retains some or all of the activity of the full-length polypeptide. For example, a functional fragment of a NS1, NS2, NS3, NS4, NS5, NS6, NS7, VP1 or VP2 can be, for example, about 20, 30, 40, 50, 60, 70, 80, 90, 100, 150, 200, 300, 350, etc., amino acids in length.

The term “HLA” as used herein refers to human leukocyte antigen. There are 7,196 HLA alleles. These are divided into 6 HLA class I and 6 HLA class II alleles for each individual (on two chromosomes). The HLA system or complex is a gene complex encoding the major histocompatibility complex (MHC) proteins in humans. HLAs corresponding to MHC Class I (A, B, or C) present peptides from within the cell and activate CD8-positive (i.e., cytotoxic) T-cells. HLAs corresponding to MHC Class II (DP, DM, DOA, DOB, DQ and DR) stimulate the multiplication of CD4-positive T-cells which stimulate antibody-producing B-cells. In some embodiments, the composition comprises a therapeutically effective amount of one or a plurality of T cell populations, at least one T cell population comprising a T cell receptor (TCR) comprising a structure that associates or targets one or a plurality of norovirus antigens and a MHC that is compatible or identical to the HLA of the subject to whom the T cell composition is administered. In some embodiments, the MHC comprises a MHC DP, DM, DOA, DOB, DQ or DR.

The term “isolated” as used herein means separated from components in which a material is ordinarily associated with, for example, an isolated cord blood mononuclear cell can be separated from red blood cells, plasma, and other components of cord blood.

A “naïve” T-cell or other immune effector cell as used herein is one that has not been exposed to or primed by an antigen or to an antigen-presenting cell presenting a peptide antigen capable of activating that cell.

The term “non-engineered” when referring to the cells of the compositions means a cell that does not contain or express an exogenous nucleic acid or amino acid sequence. For example, the cells of the compositions do not express, for example, a chimeric antigen receptor.

A “peptide library” or “overlapping peptide library” as used herein within the meaning of the application is a complex mixture of peptides which in the aggregate covers the partial or complete sequence of a protein antigen. Successive peptides within the mixture overlap each other, for example, a peptide library may be constituted of peptides 15 amino acids in length which overlapping adjacent peptides in the library by 11 amino acid residues and which span the entire length of a protein antigen. Peptide libraries may be commercially available or may be custom-made for particular antigens.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Percent identity” between a query nucleic acid sequence and a subject nucleic acid sequence is the“Identities” value, expressed as a percentage, that is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off. Importantly, a query nucleic acid sequence may be described by a nucleic acid sequence identified in one or more claims herein.

“Percent identity” between a query amino acid sequence and a subject amino acid sequence is the“Identities” value, expressed as a percentage, that is calculated by the BLASTP algorithm when a subject amino acid sequence has 100% query coverage with a query amino acid sequence after a pair-wise BLASTP alignment is performed. Such pair wise BLASTP alignments between a query amino acid sequence and a subject amino acid sequence are performed by using the default settings of the BLASTP algorithm available on the National Center for Biotechnology Institute's website with the filter for low complexity regions turned off Importantly, a query amino acid sequence may be described by an amino acid sequence identified in one or more claims herein. The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

A “peripheral blood mononuclear cell” or “PBMC” as used herein is any peripheral blood cell having a round nucleus. These cells consist of lymphocytes (T-cells, B-cells, NK cells) and monocytes. In humans, lymphocytes make up the majority of the PBMC population, followed by monocytes, and a small percentage of dendritic cells.

The term “precursor cell” as used herein refers to a cell which can differentiate or otherwise be transformed into a particular kind of cell. For example, a “T-cell precursor cell” can differentiate into a T-cell and a “dendritic precursor cell” can differentiate into a dendritic cell.

A “subject” or “host” as used herein is a vertebrate, preferably a mammal, more preferably a human. Mammals include, but are not limited to humans, simians, equines, bovines, porcines, canines, felines, murines, other farm animals, sport animals, or pets.

A “patient” or “subject in need thereof” as used herein includes those in need of virus- or other antigen-specific T-cells, such as those with lymphocytopenia, those who have undergone immune system ablation, those with primary immunodeficiency disorders, those undergoing transplantation and/or immunosuppressive regimens, those having naïve or developing immune systems, such as neonates, or those undergoing cord blood or stem cell transplantation. In a typical embodiment, the term “patient” or “subject in need thereof” as used herein refers to a human.

A “T-cell population” or “T-cell subpopulation” can include thymocytes, immature T-lymphocytes, mature T-lymphocytes, resting T-lymphocytes and activated T-lymphocytes. The T-cell population or subpopulation can include αβ T-cells, including CD4+ T-cells, CD8+ T cells, γδ T-cells, Natural Killer T-cells, or any other subset of T-cells.

The term “disclosed composition” refers to a multiple single norovirus antigen T-cell composition. In some embodiments, the disclosed composition is comprised of two or more T-cell subpopulations, wherein each T-cell subpopulation targets a single norovirus antigen. For purposes herein, when referring to combining T-cell subpopulations to comprise the disclosed composition, combining is intended to include the situation wherein the T-cells are physically combined into a single dosage form, that is, a single composition. In alternative embodiments, the T-cell subpopulations are kept physically separated but administrated concomitantly and collectively comprise the disclosed composition.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. In some embodiments, the disclosed compositions are administered with at least one pharmaceutically acceptable carrier.

The term “pharmaceutically acceptable carrier” is art recognized and includes a pharmaceutically acceptable material, composition or vehicle, suitable for administering compositions of the present disclosure to subjects. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include, but not limited to, sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Suitable pharmaceutical carriers are described in “Remington's Pharmaceutical Sciences” by E. W. Martin, which is incorporated herein by reference in its entirety. In some embodiments, the pharmaceutically acceptable carrier is sterile and pyrogen-free water. In some embodiments, the pharmaceutically acceptable carrier is Ringer's Lactate, sometimes known as lactated Ringer's solution.

The term“T cell receptor” (“TCR”) as used herein, refers to the receptor present on the surface of T cells which recognizes fragments of antigen as peptides bound to major histocompatibility complex (MHC) molecules. Native TCRs exist in ab and gd forms, which are structurally similar but exist in different locations and are thought to have different functions. The extracellular portion of the TCR has two constant domains and two variable domains. The variable domains contain polymorphic loops which form the binding site of the TCR and are analogous to complementarity determining regions (CDRs) in antibodies. In the context of cell immunotherapies, the TCR is usually genetically modified to change or improve its antigen recognition. For example, WO01/055366 and WO2006/000830, which are herein incorporated by reference in their entireties, describe retrovirus-based methods for transfecting T cells with heterologous TCRs. WO2005/113595, which is herein incorporated by reference in its entirety, describes high affinity NY-ESO T cell receptors.

Suitable TCRs bind specifically to a major histocompatibility complex (MHC) on the surface of cancer cells that displays a peptide fragment of a tumor antigen. An MHC is a set of cell surface proteins which allow the acquired immune system to recognise ‘foreign’ molecules. Proteins are intracellularly degraded and presented on the surface of cells by the MHC. MHCs displaying ‘foreign’ peptides, such a viral or cancer associated peptides, are recognised by T cells with the appropriate TCRs, prompting cell destruction pathways. MHCs on the surface of cancer cells may display peptide fragments of tumor antigen i.e. an antigen which is present on a cancer cell but not the corresponding non-cancerous cell. T cells which recognise these peptide fragments may exert a cytotoxic effect on avirally infected cell. the TCR is not naturally expressed by the T cells (i.e. the TCR is exogenous or heterologous). Heterologous TCRs may include ab TCR heterodimers. Suitable heterologous TCRs may bind specifically to cancer cells that express a tumor antigen. For example, the T cells may be modified to express a heterologous TCR that binds specifically to MHCs displaying peptide fragments of a tumor antigen expressed by the cancer cells in a specific cancer patient. Tumor antigens expressed by cancer cells in the cancer patient may identified using standard techniques. A heterologous TCR may be a synthetic or artificial TCR, i.e., a TCR that does not exist in nature. For example, a heterologous TCR may be engineered to increase its affinity or avidity for a tumor antigen (i.e. an affinity enhanced TCR). The affinity enhanced TCR may comprise one or more mutations relative to a naturally occurring TCR, for example, one or more mutations in the hypervariable complementarity determining regions (CDRs) of the variable regions of the TCR a and b chains. These mutations increase the affinity of the TCR for MHCs that display a peptide fragment of a tumor antigen expressed by cancer cells. Suitable methods of generated affinity enhanced TCRs include screening libraries of TCR mutants using phage or yeast display and are well known in the art (see for example Robbins et al J Immunol (2008) 180(9):6116; San Miguel et al (2015) Cancer Cell 28 (3) 281-283; Schmitt et al (2013) Blood 122 348-256; Jiang et al (2015) Cancer Discovery 5 901), which are incorporated by reference in their entireties.

The terms “binding to or targeting” means the magnitude of association between two amino acid sequences through either non-covalent binding. In some embodiments, the binding or targeting is measured by calculating the disassociation constant as between the amino acid sequences. In the case of this disclosure and in some embodiments, the population of T cells comprises a TCR that binds to a target sequence on the surface of target cells, such as cells infected and expressing viral antigens. The T cell or population of T cells according to the present disclosure may comprise a heterologous TCR which may specifically bind and/or bind with high affinity to the cancer or tumour antigen or peptide thereof, peptide, antigenic peptide, peptide fragment of a cancer or tumour antigen or presented by tumour of cancer cell or tissue and recognised by the heterologous TCR optionally in complex with HLA. According to the invention the heterologous TCR may bind with a dissociation constant of from about 0.01 mM and to about 100 mM, from about 0.01 mM to about 50 pM, from about 0.01 pM to about 20 pM, from about 0.05 pM to about 20 pM or about 0.01, 0.02, 0.03, 0.04, 0.05, 0.06, 0.07, 0.08, 0.09, 0.1 pM, 0.15 pM, 0.2 pM, 0.25 pM, 0.3 pM, 0.35 pM, 0.4 pM, 0.45 pM, 0.5 pM, 0.55 pM, 0.6 pM, 0.65 pM, 0.7 pM, 0.75 pM, 0.8 pM, 0.85 pM, 0.9 pM, 0.95 pM, 1.0 pM, 1.5 pM, 2.0 pM, 2.5 pM, 3.0 mM, 3.5 mM, 4.0 mM, 4.5 mM, 5.0 mM, 5.5 mM, 6.0 mM, 6.5 mM, 7.0 mM, 7.5 mM, 8.0 mM, 8.5 mM, 9.0 mM, 9.5 mM, or about 10.0 mM; from about 10 pM to about 1000 mM, from about 10 mM to about 500 mM, from about 50 mM to about 500 mM or to about 10, 20 30, 40, 50 60, 70, 80, 90 mM, 100 mM, 150 mM, 200 mM, 250 mM, 300 mM, 350 mM, 400 mM, 450 mM, 500 mM; optionally measured with surface plasmon resonance, optionally at 25° C., optionally between a pH of 7.0 and 7.5. The dissociation constant, KD or koff/kon may be determined by experimentally measuring the dissociation rate constant, k0t, and the association rate constant, kon. A dissociation constant may be measured using a soluble form of the TCR, wherein the TCR comprises a TCR alpha chain variable domain and a TCR beta chain variable domain. Accordingly, a heterologous TCR in accordance with the invention is capable of binding efficiently and/or with high affinity to HLA displaying GVYDGREHTV for example in complex with HLA-A*02 or HLA-A*0201 for example with a dissociation constant of from about 0.01 mM to about 100 mM such as 50 mM, 100 mM, 200 mM, 500 mM. IN some embodiments, the dissociation constant is from about 0.05 mM to about 20.0 mM.

According to the invention, the heterologous TCR may selectively bind to a cancer or tumour antigen or peptide thereof, peptide, antigenic peptide or peptide fragment of an antigen preferably a cancer or tumour antigen, optionally presented on HLA (pHLA), preferably expressed by a tumour cell or a cancer cell or tissue. Selective binding denotes that the heterologous TCR binds with greater affinity to one peptide, antigenic peptide or peptide fragment of an antigen preferably a cancer or tumour antigen, optionally presented on HLA (pHLA) in comparison to another. According to the present invention the binding is selective and/or specific for a viral or antigen or peptide thereof which may be a norovirus antigen, NY-ESO-1, MART-1 (melanoma antigen recognized by T cells), WT1 (Wilms tumor 1), gp100 (glycoprotein 100), tyrosinase, PRAME (preferentially expressed antigen in melanoma), p53, HPV-E6/HPV-E7 (human papillomavirus), HBV, TRAIL, DR4, Thyroglobin, TGFBII frameshift antigen, LAGE-1A, KRAS, CMV (cytomegalovirus), CEA (carcinoembryonic antigen), AFP (a-fetoprotein), MAGE-A1, MAGE-A2, MAGE-A3, MAGE-A4, MAGE-A6, MAGE-A8, and MAGE-A9, MAGE-A10, or MAGE-A12. Additionally and/or alternatively the selectivity of binding may be for HLA type i.e. to HLAs corresponding to MHC class I (A, B, and C) which all are the HLA Class1 or specific alleles thereof or HLAs corresponding to MHC class II (DP, DM, DO, DQ, and DR) or specific alleles thereof, preferably the HLA is class 1, preferably the allele is HLA-A2 or HLA-A*02 or an HLA-A2+ or HLA-A*02 positive HLA, preferably HLA-*0201. Suitable TCRs bind specifically to a major histocompatibility complex (MHC) on the surface of tumour or cancer cells that displays a peptide fragment of a tumour antigen. An MHC is a set of cell-surface proteins which allow the acquired immune system to recognise ‘foreign’ molecules. Proteins are intracellularly degraded and presented on the surface of cells by the MHC. MHCs displaying‘foreign’ peptides, such a viral-associated peptides, are recognized by T cells with the appropriate TCRs, prompting cell destruction pathways. MHCs on the surface of virally-infected cells may display peptide fragments of the viral antigen (i.e. an antigen which is present on a virally infected cell but not the corresponding uninfected cell. T cells which recognize these peptide fragments may exert a cytotoxic effect on the tumour or cancer cell.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Norovirus Specific Antigens

T-cell subpopulations targeting a single norovirus antigen can be prepared by pulsing antigen presenting cells or artificial antigen presenting cells with a single peptide or epitope, several peptides or epitopes, or with overlapping peptide libraries of the selected antigen, that for example, include peptides that are about 7, 8, 9, 10, 11, 12, 13, 14, 15, 16 or more amino acids long and overlapping one another by 5, 6, 7, 8, 9, 10, 11 or more amino acids, in certain aspects.

In particular embodiments, the peptides are 7, 8, 9, 10, 11, 12, 13, 14, 15, 16, 17, 18, 19, 20, 21, 22, 23, 24, 25, 26, 27, 28, 29, 30, 31, 32, 33, 34, or 35 or more amino acids in length, for example, and there is overlap of 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length.

In some embodiments, the disclosed composition includes norovirus specific T-cells. Norovirus specific T-cells can be generated as described below using one or more antigenic peptides to norovirus. In some embodiments, the norovirus specific T-cells are generated using one or more antigenic peptides to norovirus, or a modified or heteroclitic peptide derived from a norovirus antigenic peptide. In some embodiments, norovirus specific T-cells are generated using a norovirus antigen library comprising a pool of peptides (for example, amino acid sequences that are from about 10 to about 20 amino acids from one or a combination of sequence identifiers disclosed herein, such as 15mers containing amino acid overlap (for example 11 amino acids of overlap) between each sequence formed by scanning the protein having the amino acid sequence of SEQ ID NO: 1 (non-structural polyprotein of norovirus strain Hu/GII.4/Sydney/NSW0514/2012/AU; GenBank: JX459908):

MKMASNDASAAAVANSNNDIAKSSSDGVLSNMAVTFKRALGARPKQPPP
KEIPPRPPRPPTPELVKKIPPPPPNGEDELVVSYSAKDGVSGLPELTTV
RQPEETNTAFSVPPLNQRESRDAKEPLTGTIIEMWDGEIYHYGLYVERG
LILGVHKPPAAISLAKVELAPLSLFWRPVYTPQYLISPDTLRRLHGESF
PYTAFDNNCYAFCCWVLDLNDSWLSRRMIQRTTGFFRPYQDWNRKPLPT
MDDSKLKKVANIFLCTLSSLFTRPIKDIIGKLKPLNILNILATCDWTFA
GIVESLILLAELFGVFWTPPDVSAMIAPLLGDYELQGPEDLAVELVPIV
MGGIGLVLGFTKEKIGKMLSSAASTLRACKDLGAYGLEILKLVMKWFFP
KKEEANELAMVRSIEDAVLDLEAIENNHMTTLLKDKDSLATYMRTLDLE
EEKARKLSTKSASPDIVGTINSLLARIAAARSLVHRAKEELSSRPRPVV
VMISGKPGIGKTHLARELAKKIAASLTGDQRVGLIPRNGVDHWDAYKGE
RVVLWDDYGMSNPIHDALRLQELADTCPLTLNCDRIENKGKVFDSDAII
ITTNLANPAPLDYVNFEACSRRIDFLVYAEAPEVEKAKRDFPGQPDMWK
NAFSPDFSHIKLSLAPQGGFDKNGNTPHGKGVMKTLTTGSLIARASGLL
HERLDEYELQGPALTTFNFDRNKILAFRQLAAENKYGLMDTMRVGKQLK
DVKTMSDLKQALKNIAIKKCQIVYNGSTYTLEADGKGSVKVDKVQSAIV
QTNNELAGALHHLRCARIRYYVKCVQEALYSIIQIAGAAFVTTRIAKRM
NIQNLWSKPQVEDTEEMANKDGCLKPKDDEEFVVSSDDIKTEGKKGKNK
SGRGKKHTAFSSKGLSDEEYDEYKRIREERNGKYSIEEYLQDRDRYYEE
VAIARATEEDFCEEEEAKIRQRIFRPTRKQRKEERASLGLVTGSEIRKR
NPEDFKPKGKLWADDDRSVDYNEKLNFEAPPSIWSRIVNFGSGWGFWVS
PSLFITSTHVIPQSAKEFFGVPIKQIQIHKSGEFCRLRFPKPIRTDVTG
MILEEGAPEGTVATLLIKRPTGELMPLAARMGTHATMKIQGRTVGGQMG
MLLTGSNAKSMDLGTTPGDCGCPYIYKRGNDYVVIGVHTAAARGGNTVI
CATQGSEGEATLEGGDSKGTYCGAPILGPGSAPKLSTKTKFWRSSTTPL
PPGTYEPAYLGGKDPRVKGGPSLQQVMRDQLKPFTEPRGKPPRPNVLEA
AKKTIINVLEQTIDPPQKWSFAQACASLDKTTSSGHPHHMRKNDCWNGE
SFTGKLADQASKANLMFEEGKSMTPVYTGALKDELVKTDKVYGKVKKRL
LWGSDLATMIRCARAFGGLMDELKAHCVTLPVRVGMNMNEDGPIIFEKH
SRYRYHYDADYSRWDSTQQRDVLAAALEIMVKFSPEPHLAQIVAEDLLS
PSVMDVGDFQISISEGLPSGVPCTSQWNSIAHWLLTLCALSEVTDLSPD
IIQANSLFSFYGDDEIVSTDIKLDPEKLTAKLKEYGLKPTRPDKTEGPL
VISEDLDGLTFLRRTVTRDPAGWFGKLEQSSILRQMYWTRGPNHEDPFE
TMIPHSQRPIQLMSLLGEAALHGPAFYSKISKLVIAELKEGGMDFYVPR
QEPMFRWMRFSDLSTWEGDRNLAPSFVNEDGVE

In some embodiments, norovirus specific T-cells are generated using a norovirus antigen library comprising a pool of peptides (for example 15mers) containing amino acid overlap (for example 11 amino acids of overlap) between each sequence formed by scanning the protein having the amino acid sequence of SEQ ID NO: 2 (norovirus protease NS1-2):

RESRDAKEPLTGTIIEMWDGEIYHYGLYVERGLILGVHKPPAAISLAKV
ELAPLSLFWRPVYTPQYLISPDTLRRLHGESFPYTAFDNNCYAFCCWVL
DLNDSWLSRRMIQRTTGFFRPYQDWNRKPLPTMDDSKLKKVANIFLCTL
SSLFTRPIKDIIGKLKPLNILNILATCDWTFAGIVESLILLAELFGVFW
TPPDVSAMIAPLLGDYELQGPEDLAVELVPIVMGGIGLVLGFTKEKIGK
MLSSAASTLRACKDLGAYGLEILKLVMKWFFPKKEEANELAMVRSIEDA
VLDLEAIENNHMTTLLKDKDSLATYMRTLDLEEEKARKLSTKSASPDIV
GTINSLLARIAAARS

In some embodiments, norovirus specific T-cells are generated using a norovirus antigen library comprising a pool of peptides (for example 15mers) containing amino acid overlap (for example 11 amino acids of overlap) between each sequence formed by scanning the protein having the amino acid sequence of SEQ ID NO: 3 (norovirus protease NS3):

VMISGKPGIGKTHLARELAKKIAASLTGDQRVGLIPRNGVDHWDAYKG
ERVVLWDDYGMSNPIHDALRLQELADTCPLTLNCDRIENKGKVFDSDA
IIITTN

In some embodiments, norovirus specific T-cells are generated using a norovirus antigen library comprising a pool of peptides (for example 15mers) containing amino acid overlap (for example 11 amino acids of overlap) between each sequence formed by scanning the protein having the amino acid sequence of SEQ ID NO: 4 (norovirus protease NS5):

SPDFSHIKLSLAPQGGFDKNGNTPHGKGVMKTLTTGSLIARASGLLHER
LDEYELQGPALTTFNFDRNKILAFRQLAAENKYGLMDTMRVGKQLKDVK
TMSDLKQALKNIAIKKCQIVYNGSTYTLEADGKGSVKVDKVQSAIVQTN
NELAGALHHLRCARIRYYVKCVQEALYSIIQIAGAAFVTTRIAKRMNIQ
NLWSKPQVEDTEEMANKDGCLKPKDDEEFVVSSDDIKTEGKKGKNKSGR
GKKHTAFSSKGLSDEEYDEYKRIREERNGKYSIEEYLQDRDRYYEEVAI
ARATEEDFCEEEEAKIRQRIFRPTRKQRKEERASLGLVTGSEIRKRNPE
DFKPKGKLWADDDRSVDYNEKLNFE

In some embodiments, norovirus specific T-cells are generated using a norovirus antigen library comprising a pool of peptides (for example 15mers) containing amino acid overlap (for example 11 amino acids of overlap) between each sequence formed by scanning the protein having the amino acid sequence of SEQ ID NO: 5 (norovirus protease NS6):

APPSIWSRIVNFGSGWGFWVSPSLFITSTHVIPQSAKEFFGVPIKQIQI
HKSGEFCRLRFPKPIRTDVTGMILEEGAPEGTVATLLIKRPTGELMPLA
ARMGTHATMKIQGRTVGGQMGMLLTGSNAKSMDLGTTPGDCGCPYIYKR
GNDYVVIGVHTAAARGGNTVICATQGSEGEATLE

In some embodiments, norovirus specific T-cells are generated using a norovirus antigen library comprising a pool of peptides (for example 15mers) containing amino acid overlap (for example 11 amino acids of overlap) between each sequence formed by scanning the protein having the amino acid sequence of SEQ ID NO: 6 (norovirus protease NS7):

PKLSTKTKFWRSSTTPLPPGTYEPAYLGGKDPRVKGGPSLQQVMRDQLK
PFTEPRGKPPRPNVLEAAKKTIINVLEQTIDPPQKWSFAQACASLDKTT
SSGHPHHMRKNDCWNGESFTGKLADQASKANLMFEEGKSMTPVYTGALK
DELVKTDKVYGKVKKRLLWGSDLATMIRCARAFGGLMDELKAHCVTLPV
RVGMNMNEDGPIIFEKHSRYRYHYDADYSRWDSTQQRDVLAAALEIMVK
FSPEPHLAQIVAEDLLSPSVMDVGDFQISISEGLPSGVPCTSQWNSIAH
WLLTLCALSEVTDLSPDIIQANSLFSFYGDDEIVSTDIKLDPEKLTAKL
KEYGLKPTRPDKTEGPLVISEDLDGLTFLRRTVTRDPAGWFGKLEQSSI
LRQMYWTRGPNHEDPFETMIPHSQRPIQLMSLLGEAALHGPAFYSKISK
LVIAELKEGGMDFYVPRQEPMFRWMR

In some embodiments, norovirus specific T-cells are generated using a norovirus antigen library comprising a pool of peptides (for example 15mers) containing amino acid overlap (for example 11 amino acids of overlap) between each sequence formed by scanning the protein having the amino acid sequence of SEQ ID NO: 7 (norovirus major capsid protein VP1):

MKMASSDANPSDGSAANLVPEVNNEVMALEPVVGAAIAAPVAGQQNVID
PWIRNNFVQAPGGEFTVSPRNAPGEILWSAPLGPDLNPYLSHLARMYNG
YAGGFEVQVILAGNAFTAGKVIFAAVPPNFPTEGLSPSQVTMFPHIVVD
VRQLEPVLIPLPDVRNNFYHYNQSNDPTIKLIAMLYTPLRANNAGDDVF
TVSCRVLTRPSPDFDFIFLVPPTVESRTKPFSVPVLTVEEMTNSRFPIP
LEKLFTGPSSAFVVQPQNGRCTTDGVLLGTTQLSPVNICTFRGDVTHIT
GSRNYTMNLASQNWNDYDPTEEIPAPLGTPDFVGKIQGVLTQTTRTDGS
TRGHKATVYTGSADFAPKLGRVQFETDTDRDFEANQNTKFTPVGVIQDG
GTTHRNEPQQWVLPSYSGRNTHNVHLAPAVAPTFPGEQLLFFRSTMPGC
SGYPNMDLDCLLPQEWVQYFYQEAAPAQSDVALLRFVNPDTGRVLFECK
LHKSGYVTVAHTGQHDLVIPPNGYFRFDSWVNQFYTLAPMGNGTGRRRA
V

In some embodiments, norovirus specific T-cells are generated using a norovirus antigen library comprising a pool of peptides (for example 15mers) containing amino acid overlap (for example 11 amino acids of overlap) between each sequence formed by scanning the protein having the amino acid sequence of SEQ ID NO: 8 (norovirus minor capsid protein VP2):

MAGAFFAGLASDVLGSGLGSLINAGAGAINQKVEFENNRKLQQASFQFS
SNLQQASFQHDKEMLQAQIEATKKLQQEMMKVKQAMLLEGGFFETDAAR
GAINAPMTKALDWSGTRYWAPDARTTTYNAGRFSTPQPSGALPGRANLR
DAVPARGSSSKSSNSSTATSVYSNQTTSTRLGSTAVSGTSVSSFPSTAR
TRSWVEDQSRNLSPSMRGAHNISFVTPPSSRSSSQGTVSTVPKEVLDSW
TGAFNTRRQPLFAHIRKRGESRA

The antigenic peptide library can be obtained commercially, for example, by customly ordering from A&A Labs (San Diego, Calif.). In some embodiments, the norovirus specific T-cells are generated using an overlapping antigenic library made up of the entire norovirus polyprotein. In some embodiments, the norovirus specific T-cells are generated using an overlapping antigenic library made up of less than the entire norovirus polyprotein.

In some embodiments, the norovirus specific T-cells are generated using one or more antigenic peptides of a norovirus polyprotein, or a modified or heteroclitic peptide derived from a norovirus peptide. In some embodiments, the norovirus specific T-cells are generated with peptides that recognize class I MEW molecules. In some embodiments, the norovirus specific T-cells are generated with peptides that recognize class II MHC molecules. In some embodiments, the norovirus specific T-cells are generated with peptides that recognize both class I and class II MEW molecules.

In some embodiments, the norovirus peptides used to prime and expand a T-cell subpopulation includes specifically selected HLA-restricted peptides generated by determining the HLA profile of the donor source, and including peptides derived from norovirus that best match the donor's HLA. In some embodiments, the norovirus peptides used to prime and expand a T-cell subpopulation are derived from HLA-restricted peptides selected from at least one or more of an HLA-A restricted peptide, HLA-B restricted peptide, or HLA-DR restricted peptide. Suitable methods for generating HLA-restricted peptides from an antigen have been described in, for example, Rammensee, H G., Bachmann, J., Emmerich, N. et al., SYFPEITHI: database for MHC ligands and peptide motifs. Immunogenetics (1999) 50: 213 (available at doi.org/10.1007/s002510050595).

As provided herein, the HLA profile of a donor cell source can be determined, and T-cell subpopulations targeting norovirus derived, wherein the T-cell subpopulation is primed and expanded using a group of peptides that are HLA-restricted to the donor's HLA profile. In certain embodiments, the T-cell subpopulation is exposed to a peptide mix that includes one or more HLA-A restricted, HLA-B restricted, and HLA-DR restricted peptides. In certain embodiments, the T-cell subpopulation is exposed to a peptide mix that includes HLA-A restricted, HLA-B restricted, and HLA-DR restricted peptides. For example, if the donor cell source has an HLA profile that is HLA-A*01/*02:01; HLA-B*15:01/*18; and HLA-DRB1*0101/*0301, then the norovirus peptides used to prime and expand the norovirus specific T-cell subpopulation are restricted to the specific HLA profile, and may include the peptides for HLA-A*01, the peptides for HLA-A*02:01, the peptides for HLA-B*15:01/*18, and the peptides for HLA-DRB1*0101/*0301. In some embodiments, the mastermix of peptides includes both an overlapping peptide library and specifically selected HLA-restricted peptides generated by determining the HLA profile of the donor source.

### T-Cell Compositions

The T-cell composition according to the present disclosure can comprise one or a plurality of T-cell subpopulations and each T-cell subpopulation is specific for one or a plurality of norovirus antigens. In some embodiments, the disclosed composition comprises only one T-cell subpopulation specific for a norovirus antigen. In some embodiments, the disclosed composition comprises a first T-cell subpopulation and a second T-cell subpopulation, wherein each T-cell subpopulation is specific for a different norovirus antigen. In some embodiments, the disclosed composition comprises a first T-cell subpopulation, a second T-cell subpopulation, and a third T-cell subpopulation, wherein each T-cell subpopulation is specific for a different norovirus antigen. In some embodiments, the disclosed composition comprises a first T-cell subpopulation, a second T-cell subpopulation, a third T-cell subpopulation, and a fourth T-cell subpopulation, wherein each T-cell subpopulation is specific for a different norovirus antigen. In some embodiments, the disclosed composition comprises up to about five T-cell subpopulations and each T-cell subpopulation is specific for a different norovirus antigen. In some embodiments, the disclosure relates to a library of T-cells specific for one or a plurality of norovirus antigens, such library being stored frozen for no less than about 1, 10, 15, 30, 45, 90, 120, 150, 180 or more days.

The ratio of the T-cell subpopulations in the disclosed composition is selected, in some embodiments, based on the knowledge of the patient's viral load or the healthcare provider's best judgment. In some embodiments, each of the T-cell subpopulations in the disclosed composition is in a defined ratio based on either total cell number or normalized cell activity. In some embodiments, each of the T-cell subpopulations in the disclosed composition is in about an equal ratio. In some embodiments, the ratio or percentage of each T-cell subpopulation is normalized based on the measured activity of each T-cell subpopulation against the norovirus as measured by, for example, but not limited to, the Eli Spot assay.

In some embodiments, the disclosed composition comprises a first T-cell subpopulation and a second T-cell subpopulation, wherein the first and second T-cell subpopulations are in about an equal ratio based on either total cell number or normalized cell activity. In some embodiments, the disclosed composition comprises a first T-cell subpopulation and a second T-cell subpopulation, wherein the percentage of the first subpopulation in the disclosed composition is from about 40% to about 60% of the disclosed composition based on either total cell number or normalized cell activity. In some embodiments, the disclosed composition comprises three T-cell subpopulations, wherein the disclosed composition comprises (i) at least about 45%, 50%, 55%, 60%, 65%, 70%, 75%, 80%, or 85% of a first T-cell subpopulation, (ii) at least about 5%, 10%, 15%, 20%, or 25% of a second T-cell subpopulation, and (iii) at least about 10%, 15%, 20%, 25%, 30%, or 35% of a third T-cell subpopulation, wherein the percentage adds to 100% based on either total cell number or normalized cell activity. The ratios of the T-cell subpopulations in the disclosed composition may be selected based on the knowledge of the patient's infecting norovirus or the healthcare provider's best judgement. In some embodiments, the disclosed composition comprises four T-cell subpopulations, wherein the disclosed composition comprises (i) at least about 45%, 50%, 55%, 60%, 65%, 70%, 75%, 80%, or 85% of a first T-cell subpopulation, (ii) at least about 5%, 10%, 15%, 20%, 25%, 30%, 35%, 40% or 45% of a second T-cell subpopulation, (iii) at least about 5%, 10%, 15%, 20%, 25%, 30%, 35%, 40% or 45% of a third T-cell subpopulation, and (iv) at least about 5%, 10%, 15%, 20%, 25%, 30%, 35%, 40% or 45% of a fourth T-cell subpopulation, wherein the percentage adds to 100% based on either total cell number or normalized cell activity. In some embodiments, the disclosed composition comprises five T-cell subpopulations, wherein the disclosed composition comprises (i) at least about 45%, 50%, 55%, 60%, 65%, 70%, 75%, 80% of a first T-cell subpopulation, (ii) at least about 5%, 10%, 15%, 20%, 25%, 30%, 35%, or 40% of a second T-cell subpopulation, (iii) at least about 5%, 10%, 15%, 20%, 25%, 30%, 35%, or 40% of a third T-cell subpopulation, (iv) at least about 5%, 10%, 15%, 20%, 25%, 30%, 35%, or 40% of a fourth T-cell subpopulation, and (v) at least about 5%, 10%, 15%, 20%, 25%, 30%, 35%, or 40% of a fifth T-cell subpopulation, wherein the percentage adds to 100% based on either total cell number or normalized cell activity.

In some embodiments, the ratio or percentage of each T-cell subpopulation is normalized based on the measured activity of each T-cell subpopulation against the norovirus antigen as measured by, for example, but not limited to, the ELISpot assay. In some embodiments, the percentage of the T-cell subpopulations is based on the norovirus antigen profile of the norovirus strain found in the patient such that the percentage of each T-cell subpopulation correlates with the norovirus antigen profile of the norovirus strain found in the patient. In some embodiments, each of the T-cell subpopulations is specific to one or a combination of: NS1-2, NS3, NS4, NS5, NS6, NS7, VP1, and VP2, or a functional fragment thereof.

In some embodiments, the disclosed composition comprises at least two T-cell subpopulations, wherein the first T-cell subpopulation is specific to NS1-2, NS-1, NS2 or a functional fragment thereof, and the second T-cell subpopulation is specific to any one of NS3, NS4, NS5, NS6, NS7, VP1 and VP2, or a functional fragment thereof. In some embodiments, the disclosed composition comprises at least two T-cell subpopulations, wherein the first T-cell subpopulation is specific to NS3, or a functional fragment thereof, and the second T-cell subpopulation is specific to any one of NS4, NS5, NS6, NS7, VP1 and VP2, or a functional fragment thereof. In some embodiments, the disclosed composition comprises at least two T-cell subpopulations, wherein the first T-cell subpopulation is specific to NS4, or a functional fragment thereof, and the second T-cell subpopulation is specific to any one of NS5, NS6, NS7, VP1 and VP2, or a functional fragment thereof. In some embodiments, the disclosed composition comprises at least two T-cell subpopulations, wherein the first T-cell subpopulation is specific to NS5, or a functional fragment thereof, and the second T-cell subpopulation is specific to any one of NS6, NS7, VP1 and VP2, or a functional fragment thereof. In some embodiments, the disclosed composition comprises at least two T-cell subpopulations, wherein the first T-cell subpopulation is specific to NS6, or a functional fragment thereof, and the second T-cell subpopulation is specific to any one of NS7, VP1 and VP2, or a functional fragment thereof. In some embodiments, the disclosed composition comprises at least two T-cell subpopulations, wherein the first T-cell subpopulation is specific to NS7, or a functional fragment thereof, and the second T-cell subpopulation is specific to any one of VP1 and VP2, or a functional fragment thereof. In some embodiments, the disclosed composition comprises at least two T-cell subpopulations, wherein the first T-cell subpopulation is specific to VP1, or a functional fragment thereof, and the second T-cell subpopulation is specific to VP2, or a functional fragment thereof.

In some embodiments, the disclosed composition comprises at least three T-cell subpopulations, wherein the first T-cell subpopulation is specific to NS1-2, NS1, NS2 or a functional fragment thereof, the second T-cell subpopulation is specific to NS3, or a functional fragment thereof, and the third T-cell subpopulation is specific to any one of NS4, NS5, NS6, NS7, VP1 and VP2, or a functional fragment thereof. In some embodiments, the disclosed composition comprises at least three T-cell subpopulations, wherein the first T-cell subpopulation is specific to NS1-2, or a functional fragment thereof, the second T-cell subpopulation is specific to NS4, or a functional fragment thereof, and the third T-cell subpopulation is specific to any one of NS5, NS6, NS7, VP1 and VP2, or a functional fragment thereof. In some embodiments, the disclosed composition comprises at least three T-cell subpopulations, wherein the first T-cell subpopulation is specific to NS1-2, or a functional fragment thereof, the second T-cell subpopulation is specific to NS5, or a functional fragment thereof, and the third T-cell subpopulation is specific to any one of NS6, NS7, VP1 and VP2, or a functional fragment thereof. In some embodiments, the disclosed composition comprises at least three T-cell subpopulations, wherein the first T-cell subpopulation is specific to NS1-2, or a functional fragment thereof, the second T-cell subpopulation is specific to NS6, or a functional fragment thereof, and the third T-cell subpopulation is specific to any one of NS7, VP1 and VP2, or a functional fragment thereof. In some embodiments, the disclosed composition comprises at least three T-cell subpopulations, wherein the first T-cell subpopulation is specific to NS1-2, or a functional fragment thereof, the second T-cell subpopulation is specific to NS7, or a functional fragment thereof, and the third T-cell subpopulation is specific to any one of VP1 and VP2, or a functional fragment thereof. In some embodiments, the disclosed composition comprises at least three T-cell subpopulations, wherein the first T-cell subpopulation is specific to NS1-2, or a functional fragment thereof, the second T-cell subpopulation is specific to VP1, or a functional fragment thereof, and the third T-cell subpopulation is specific to VP2, or a functional fragment thereof. In some embodiments, the disclosed composition comprises at least three T-cell subpopulations, wherein the first T-cell subpopulation is specific to NS3, or a functional fragment thereof, the second T-cell subpopulation is specific to NS4, or a functional fragment thereof, and the third T-cell subpopulation is specific to any one of NS5, NS6, NS7, VP1 and VP2, or a functional fragment thereof. In some embodiments, the disclosed composition comprises at least three T-cell subpopulations, wherein the first T-cell subpopulation is specific to NS3, or a functional fragment thereof, the second T-cell subpopulation is specific to NS5, or a functional fragment thereof, and the third T-cell subpopulation is specific to any one of NS6, NS7, VP1 and VP2, or a functional fragment thereof. In some embodiments, the disclosed composition comprises at least three T-cell subpopulations, wherein the first T-cell subpopulation is specific to NS3, or a functional fragment thereof, the second T-cell subpopulation is specific to NS6, or a functional fragment thereof, and the third T-cell subpopulation is specific to any one of NS7, VP1 and VP2, or a functional fragment thereof. In some embodiments, the disclosed composition comprises at least three T-cell subpopulations, wherein the first T-cell subpopulation is specific to NS3, or a functional fragment thereof, the second T-cell subpopulation is specific to NS7, or a functional fragment thereof, and the third T-cell subpopulation is specific to any one of VP1 and VP2, or a functional fragment thereof. In some embodiments, the disclosed composition comprises at least three T-cell subpopulations, wherein the first T-cell subpopulation is specific to NS3, or a functional fragment thereof, the second T-cell subpopulation is specific to VP1, or a functional fragment thereof, and the third T-cell subpopulation is specific to VP2, or a functional fragment thereof. In some embodiments, the disclosed composition comprises at least three T-cell subpopulations, wherein the first T-cell subpopulation is specific to NS4, or a functional fragment thereof, the second T-cell subpopulation is specific to NS5, or a functional fragment thereof, and the third T-cell subpopulation is specific to any one of NS6, NS7, VP1 and VP2, or a functional fragment thereof. In some embodiments, the disclosed composition comprises at least three T-cell subpopulations, wherein the first T-cell subpopulation is specific to NS4, or a functional fragment thereof, the second T-cell subpopulation is specific to NS6, or a functional fragment thereof, and the third T-cell subpopulation is specific to any one of NS7, VP1 and VP2, or a functional fragment thereof. In some embodiments, the disclosed composition comprises at least three T-cell subpopulations, wherein the first T-cell subpopulation is specific to NS4, or a functional fragment thereof, the second T-cell subpopulation is specific to NS7, or a functional fragment thereof, and the third T-cell subpopulation is specific to any one of VP1 and VP2, or a functional fragment thereof. In some embodiments, the disclosed composition comprises at least three T-cell subpopulations, wherein the first T-cell subpopulation is specific to NS4, or a functional fragment thereof, the second T-cell subpopulation is specific to VP1, or a functional fragment thereof, and the third T-cell subpopulation is specific to VP2, or a functional fragment thereof. In some embodiments, the disclosed composition comprises at least three T-cell subpopulations, wherein the first T-cell subpopulation is specific to NS5, or a functional fragment thereof, the second T-cell subpopulation is specific to NS6, or a functional fragment thereof, and the third T-cell subpopulation is specific to any one of NS7, VP1 and VP2, or a functional fragment thereof. In some embodiments, the disclosed composition comprises at least three T-cell subpopulations, wherein the first T-cell subpopulation is specific to NS5, or a functional fragment thereof, the second T-cell subpopulation is specific to NS7, or a functional fragment thereof, and the third T-cell subpopulation is specific to any one of VP1 and VP2, or a functional fragment thereof. In some embodiments, the disclosed composition comprises at least three T-cell subpopulations, wherein the first T-cell subpopulation is specific to NS5, or a functional fragment thereof, the second T-cell subpopulation is specific to VP1, or a functional fragment thereof, and the third T-cell subpopulation is specific to any one of VP2, or a functional fragment thereof. In some embodiments, the disclosed composition comprises at least three T-cell subpopulations, wherein the first T-cell subpopulation is specific to NS6, or a functional fragment thereof, the second T-cell subpopulation is specific to NS7, or a functional fragment thereof, and the third T-cell subpopulation is specific to any one of VP1 and VP2, or a functional fragment thereof. In some embodiments, the disclosed composition comprises at least three T-cell subpopulations, wherein the first T-cell subpopulation is specific to NS6, or a functional fragment thereof, the second T-cell subpopulation is specific to VP1, or a functional fragment thereof, and the third T-cell subpopulation is specific to VP2 or a functional fragment thereof. In some embodiments, the disclosed composition comprises at least three T-cell subpopulations, wherein the first T-cell subpopulation is specific to NS7, or a functional fragment thereof, the second T-cell subpopulation is specific to VP1, or a functional fragment thereof, and the third T-cell subpopulation is specific to VP2, or a functional fragment thereof.

In some embodiments, the disclosed composition comprises at least four T-cell subpopulations, wherein the first T-cell subpopulation is specific to NS1-2, or a functional fragment thereof, the second T-cell subpopulation is specific to NS3, or a functional fragment thereof, the third T-cell subpopulation is specific to NS4, or a functional fragment thereof, and the fourth T-cell subpopulation is specific to any one of NS5, NS6, NS7, VP1 and VP2, or a functional fragment thereof. In some embodiments, the disclosed composition comprises at least four T-cell subpopulations, wherein the first T-cell subpopulation is specific to NS1-2, or a functional fragment thereof, the second T-cell subpopulation is specific to NS3, or a functional fragment thereof, the third T-cell subpopulation is specific to NS5, or a functional fragment thereof, and the fourth T-cell subpopulation is specific to any one of NS6, NS7, VP1 and VP2, or a functional fragment thereof. In some embodiments, the disclosed composition comprises at least four T-cell subpopulations, wherein the first T-cell subpopulation is specific to NS1-2, or a functional fragment thereof, the second T-cell subpopulation is specific to NS3, or a functional fragment thereof, the third T-cell subpopulation is specific to NS6, or a functional fragment thereof, and the fourth T-cell subpopulation is specific to any one of NS7, VP1 and VP2, or a functional fragment thereof. In some embodiments, the disclosed composition comprises at least four T-cell subpopulations, wherein the first T-cell subpopulation is specific to NS1-2, or a functional fragment thereof, the second T-cell subpopulation is specific to NS3, or a functional fragment thereof, the third T-cell subpopulation is specific to NS7, or a functional fragment thereof, and the fourth T-cell subpopulation is specific to any one of VP1 and VP2, or a functional fragment thereof. In some embodiments, the disclosed composition comprises at least four T-cell subpopulations, wherein the first T-cell subpopulation is specific to NS1-2, or a functional fragment thereof, the second T-cell subpopulation is specific to NS3, or a functional fragment thereof, the third T-cell subpopulation is specific to VP1, or a functional fragment thereof, and the fourth T-cell subpopulation is specific to VP2, or a functional fragment thereof. In some embodiments, the disclosed composition comprises at least four T-cell subpopulations, wherein the first T-cell subpopulation is specific to NS1-2, or a functional fragment thereof, the second T-cell subpopulation is specific to NS4, or a functional fragment thereof, the third T-cell subpopulation is specific to NS5, or a functional fragment thereof, and the fourth T-cell subpopulation is specific to any one of NS6, NS7, VP1 and VP2, or a functional fragment thereof In some embodiments, the disclosed composition comprises at least four T-cell subpopulations, wherein the first T-cell subpopulation is specific to NS1-2, or a functional fragment thereof, the second T-cell subpopulation is specific to NS4, or a functional fragment thereof, the third T-cell subpopulation is specific to NS6, or a functional fragment thereof, and the fourth T-cell subpopulation is specific to any one of NS7, VP1 and VP2, or a functional fragment thereof. In some embodiments, the disclosed composition comprises at least four T-cell subpopulations, wherein the first T-cell subpopulation is specific to NS1-2, or a functional fragment thereof, the second T-cell subpopulation is specific to NS4, or a functional fragment thereof, the third T-cell subpopulation is specific to NS7, or a functional fragment thereof, and the fourth T-cell subpopulation is specific to any one of VP1 and VP2, or a functional fragment thereof. In some embodiments, the disclosed composition comprises at least four T-cell subpopulations, wherein the first T-cell subpopulation is specific to NS1-2, or a functional fragment thereof, the second T-cell subpopulation is specific to NS4, or a functional fragment thereof, the third T-cell subpopulation is specific to VP1, or a functional fragment thereof, and the fourth T-cell subpopulation is specific to VP2, or a functional fragment thereof. In some embodiments, the disclosed composition comprises at least four T-cell subpopulations, wherein the first T-cell subpopulation is specific to NS1-2, or a functional fragment thereof, the second T-cell subpopulation is specific to NS5, or a functional fragment thereof, the third T-cell subpopulation is specific to NS6, or a functional fragment thereof, and the fourth T-cell subpopulation is specific to any one of NS7, VP1 and VP2, or a functional fragment thereof. In some embodiments, the disclosed composition comprises at least four T-cell subpopulations, wherein the first T-cell subpopulation is specific to NS1-2, or a functional fragment thereof, the second T-cell subpopulation is specific to NS5, or a functional fragment thereof, the third T-cell subpopulation is specific to NS7, or a functional fragment thereof, and the fourth T-cell subpopulation is specific to any one of VP1 and VP2, or a functional fragment thereof. In some embodiments, the disclosed composition comprises at least four T-cell subpopulations, wherein the first T-cell subpopulation is specific to NS1-2, or a functional fragment thereof, the second T-cell subpopulation is specific to NS5, or a functional fragment thereof, the third T-cell subpopulation is specific to VP1, or a functional fragment thereof, and the fourth T-cell subpopulation is specific to VP2, or a functional fragment thereof. In some embodiments, the disclosed composition comprises at least four T-cell subpopulations, wherein the first T-cell subpopulation is specific to NS1-2, or a functional fragment thereof, the second T-cell subpopulation is specific to NS6, or a functional fragment thereof, the third T-cell subpopulation is specific to NS7, or a functional fragment thereof, and the fourth T-cell subpopulation is specific to any one of VP1 and VP2, or a functional fragment thereof. In some embodiments, the disclosed composition comprises at least four T-cell subpopulations, wherein the first T-cell subpopulation is specific to NS1-2, or a functional fragment thereof, the second T-cell subpopulation is specific to NS6, or a functional fragment thereof, the third T-cell subpopulation is specific to VP1, or a functional fragment thereof, and the fourth T-cell subpopulation is specific to VP2, or a functional fragment thereof. In some embodiments, the disclosed composition comprises at least four T-cell subpopulations, wherein the first T-cell subpopulation is specific to NS1-2, or a functional fragment thereof, the second T-cell subpopulation is specific to NS7, or a functional fragment thereof, the third T-cell subpopulation is specific to VP1, or a functional fragment thereof, and the fourth T-cell subpopulation is specific to VP2, or a functional fragment thereof. In some embodiments, the disclosed composition comprises at least four T-cell subpopulations, wherein the first T-cell subpopulation is specific to NS3, or a functional fragment thereof, the second T-cell subpopulation is specific to NS4, or a functional fragment thereof, the third T-cell subpopulation is specific to NS5, or a functional fragment thereof, and the fourth T-cell subpopulation is specific to any one of NS6, NS7, VP1 and VP2, or a functional fragment thereof. In some embodiments, the disclosed composition comprises at least four T-cell subpopulations, wherein the first T-cell subpopulation is specific to NS3, or a functional fragment thereof, the second T-cell subpopulation is specific to NS4, or a functional fragment thereof, the third T-cell subpopulation is specific to NS6, or a functional fragment thereof, and the fourth T-cell subpopulation is specific to any one of NS7, VP1 and VP2, or a functional fragment thereof. In some embodiments, the disclosed composition comprises at least four T-cell subpopulations, wherein the first T-cell subpopulation is specific to NS3, or a functional fragment thereof, the second T-cell subpopulation is specific to NS4, or a functional fragment thereof, the third T-cell subpopulation is specific to NS7, or a functional fragment thereof, and the fourth T-cell subpopulation is specific to any one of VP1 and VP2, or a functional fragment thereof. In some embodiments, the disclosed composition comprises at least four T-cell subpopulations, wherein the first T-cell subpopulation is specific to NS3, or a functional fragment thereof, the second T-cell subpopulation is specific to NS4, or a functional fragment thereof, the third T-cell subpopulation is specific to VP1, or a functional fragment thereof, and the fourth T-cell subpopulation is specific to VP2, or a functional fragment thereof. In some embodiments, the disclosed composition comprises at least four T-cell subpopulations, wherein the first T-cell subpopulation is specific to NS3, or a functional fragment thereof, the second T-cell subpopulation is specific to NS5, or a functional fragment thereof, the third T-cell subpopulation is specific to NS6, or a functional fragment thereof, and the fourth T-cell subpopulation is specific to any one of NS7, VP1 and VP2, or a functional fragment thereof. In some embodiments, the disclosed composition comprises at least four T-cell subpopulations, wherein the first T-cell subpopulation is specific to NS3, or a functional fragment thereof, the second T-cell subpopulation is specific to NS5, or a functional fragment thereof, the third T-cell subpopulation is specific to NS7, or a functional fragment thereof, and the fourth T-cell subpopulation is specific to any one of VP1 and VP2, or a functional fragment thereof. In some embodiments, the disclosed composition comprises at least four T-cell subpopulations, wherein the first T-cell subpopulation is specific to NS3, or a functional fragment thereof, the second T-cell subpopulation is specific to NS5, or a functional fragment thereof, the third T-cell subpopulation is specific to VP1, or a functional fragment thereof, and the fourth T-cell subpopulation is specific to VP2, or a functional fragment thereof. In some embodiments, the disclosed composition comprises at least four T-cell subpopulations, wherein the first T-cell subpopulation is specific to NS3, or a functional fragment thereof, the second T-cell subpopulation is specific to NS6, or a functional fragment thereof, the third T-cell subpopulation is specific to NS7, or a functional fragment thereof, and the fourth T-cell subpopulation is specific to any one of VP1 and VP2, or a functional fragment thereof. In some embodiments, the disclosed composition comprises at least four T-cell subpopulations, wherein the first T-cell subpopulation is specific to NS3, or a functional fragment thereof, the second T-cell subpopulation is specific to NS6, or a functional fragment thereof, the third T-cell subpopulation is specific to VP1, or a functional fragment thereof, and the fourth T-cell subpopulation is specific to VP2, or a functional fragment thereof. In some embodiments, the disclosed composition comprises at least four T-cell subpopulations, wherein the first T-cell subpopulation is specific to NS3, or a functional fragment thereof, the second T-cell subpopulation is specific to NS7, or a functional fragment thereof, the third T-cell subpopulation is specific to VP1, or a functional fragment thereof, and the fourth T-cell subpopulation is specific to VP2, or a functional fragment thereof. In some embodiments, the disclosed composition comprises at least four T-cell subpopulations, wherein the first T-cell subpopulation is specific to NS4, or a functional fragment thereof, the second T-cell subpopulation is specific to NS5, or a functional fragment thereof, the third T-cell subpopulation is specific to NS6, or a functional fragment thereof, and the fourth T-cell subpopulation is specific to any one of NS7, VP1 and VP2, or a functional fragment thereof. In some embodiments, the disclosed composition comprises at least four T-cell subpopulations, wherein the first T-cell subpopulation is specific to NS4, or a functional fragment thereof, the second T-cell subpopulation is specific to NS5, or a functional fragment thereof, the third T-cell subpopulation is specific to NS7, or a functional fragment thereof, and the fourth T-cell subpopulation is specific to any one of VP1 and VP2, or a functional fragment thereof. In some embodiments, the disclosed composition comprises at least four T-cell subpopulations, wherein the first T-cell subpopulation is specific to NS4, or a functional fragment thereof, the second T-cell subpopulation is specific to NS5, or a functional fragment thereof, the third T-cell subpopulation is specific to VP1, or a functional fragment thereof, and the fourth T-cell subpopulation is specific to VP2, or a functional fragment thereof. In some embodiments, the disclosed composition comprises at least four T-cell subpopulations, wherein the first T-cell subpopulation is specific to NS4, or a functional fragment thereof, the second T-cell subpopulation is specific to NS6, or a functional fragment thereof, the third T-cell subpopulation is specific to NS7, or a functional fragment thereof, and the fourth T-cell subpopulation is specific to any one of VP1 and VP2, or a functional fragment thereof. In some embodiments, the disclosed composition comprises at least four T-cell subpopulations, wherein the first T-cell subpopulation is specific to NS4, or a functional fragment thereof, the second T-cell subpopulation is specific to NS6, or a functional fragment thereof, the third T-cell subpopulation is specific to VP1, or a functional fragment thereof, and the fourth T-cell subpopulation is specific to VP2, or a functional fragment thereof. In some embodiments, the disclosed composition comprises at least four T-cell subpopulations, wherein the first T-cell subpopulation is specific to NS4, or a functional fragment thereof, the second T-cell subpopulation is specific to NS7, or a functional fragment thereof, the third T-cell subpopulation is specific to VP1, or a functional fragment thereof, and the fourth T-cell subpopulation is specific to VP2, or a functional fragment thereof. In some embodiments, the disclosed composition comprises at least four T-cell subpopulations, wherein the first T-cell subpopulation is specific to NS5, or a functional fragment thereof, the second T-cell subpopulation is specific to NS6, or a functional fragment thereof, the third T-cell subpopulation is specific to NS7, or a functional fragment thereof, and the fourth T-cell subpopulation is specific to any one of VP1 and VP2, or a functional fragment thereof. In some embodiments, the disclosed composition comprises at least four T-cell subpopulations, wherein the first T-cell subpopulation is specific to NS5, or a functional fragment thereof, the second T-cell subpopulation is specific to NS6, or a functional fragment thereof, the third T-cell subpopulation is specific to VP1, or a functional fragment thereof, and the fourth T-cell subpopulation is specific to VP2, or a functional fragment thereof. In some embodiments, the disclosed composition comprises at least four T-cell subpopulations, wherein the first T-cell subpopulation is specific to NS6, or a functional fragment thereof, the second T-cell subpopulation is specific to NS7, or a functional fragment thereof, the third T-cell subpopulation is specific to VP1, or a functional fragment thereof, and the fourth T-cell subpopulation is specific to VP2, or a functional fragment thereof.

In some embodiments, the disclosed composition comprises at least five T-cell subpopulations, wherein the first T-cell subpopulation is specific to NS1-2, or a functional fragment thereof, the second T-cell subpopulation is specific to NS3, or a functional fragment thereof, the third T-cell subpopulation is specific to NS4, or a functional fragment thereof, the fourth T-cell subpopulation is specific to NS5, or a functional fragment thereof, and the fifth T-cell subpopulation is specific to any one of NS6, NS7, VP1 and VP2, or a functional fragment thereof. In some embodiments, the disclosed composition comprises at least five T-cell subpopulations, wherein the first T-cell subpopulation is specific to NS1-2, or a functional fragment thereof, the second T-cell subpopulation is specific to NS3, or a functional fragment thereof, the third T-cell subpopulation is specific to NS4, or a functional fragment thereof, the fourth T-cell subpopulation is specific to NS6, or a functional fragment thereof, and the fifth T-cell subpopulation is specific to any one of NS7, VP1 and VP2, or a functional fragment thereof. In some embodiments, the disclosed composition comprises at least five T-cell subpopulations, wherein the first T-cell subpopulation is specific to NS1-2, or a functional fragment thereof, the second T-cell subpopulation is specific to NS3, or a functional fragment thereof, the third T-cell subpopulation is specific to NS4, or a functional fragment thereof, the fourth T-cell subpopulation is specific to NS7, or a functional fragment thereof, and the fifth T-cell subpopulation is specific to any one of VP1 and VP2, or a functional fragment thereof. In some embodiments, the disclosed composition comprises at least five T-cell subpopulations, wherein the first T-cell subpopulation is specific to NS1-2, or a functional fragment thereof, the second T-cell subpopulation is specific to NS3, or a functional fragment thereof, the third T-cell subpopulation is specific to NS4, or a functional fragment thereof, the fourth T-cell subpopulation is specific to VP1, or a functional fragment thereof, and the fifth T-cell subpopulation is specific to VP2, or a functional fragment thereof. In some embodiments, the disclosed composition comprises at least five T-cell subpopulations, wherein the first T-cell subpopulation is specific to NS1-2, or a functional fragment thereof, the second T-cell subpopulation is specific to NS3, or a functional fragment thereof, the third T-cell subpopulation is specific to NS5, or a functional fragment thereof, the fourth T-cell subpopulation is specific to NS6, or a functional fragment thereof, and the fifth T-cell subpopulation is specific to any one of NS7, VP1 and VP2, or a functional fragment thereof. In some embodiments, the disclosed composition comprises at least five T-cell subpopulations, wherein the first T-cell subpopulation is specific to NS1-2, or a functional fragment thereof, the second T-cell subpopulation is specific to NS3, or a functional fragment thereof, the third T-cell subpopulation is specific to NS5, or a functional fragment thereof, the fourth T-cell subpopulation is specific to NS7, or a functional fragment thereof, and the fifth T-cell subpopulation is specific to any one of VP1 and VP2, or a functional fragment thereof. In some embodiments, the disclosed composition comprises at least five T-cell subpopulations, wherein the first T-cell subpopulation is specific to NS1-2, or a functional fragment thereof, the second T-cell subpopulation is specific to NS3, or a functional fragment thereof, the third T-cell subpopulation is specific to NS5, or a functional fragment thereof, the fourth T-cell subpopulation is specific to VP1, or a functional fragment thereof, and the fifth T-cell subpopulation is specific to VP2, or a functional fragment thereof. In some embodiments, the disclosed composition comprises at least five T-cell subpopulations, wherein the first T-cell subpopulation is specific to NS1-2, or a functional fragment thereof, the second T-cell subpopulation is specific to NS3, or a functional fragment thereof, the third T-cell subpopulation is specific to NS6, or a functional fragment thereof, the fourth T-cell subpopulation is specific to NS7, or a functional fragment thereof, and the fifth T-cell subpopulation is specific to any one of VP1 and VP2, or a functional fragment thereof. In some embodiments, the disclosed composition comprises at least five T-cell subpopulations, wherein the first T-cell subpopulation is specific to NS1-2, or a functional fragment thereof, the second T-cell subpopulation is specific to NS3, or a functional fragment thereof, the third T-cell subpopulation is specific to NS6, or a functional fragment thereof, the fourth T-cell subpopulation is specific to VP1, or a functional fragment thereof, and the fifth T-cell subpopulation is specific to VP2, or a functional fragment thereof. In some embodiments, the disclosed composition comprises at least five T-cell subpopulations, wherein the first T-cell subpopulation is specific to NS1-2, or a functional fragment thereof, the second T-cell subpopulation is specific to NS3, or a functional fragment thereof, the third T-cell subpopulation is specific to NS7, or a functional fragment thereof, the fourth T-cell subpopulation is specific to VP1, or a functional fragment thereof, and the fifth T-cell subpopulation is specific to VP2, or a functional fragment thereof. In some embodiments, the disclosed composition comprises at least five T-cell subpopulations, wherein the first T-cell subpopulation is specific to NS1-2, or a functional fragment thereof, the second T-cell subpopulation is specific to NS4, or a functional fragment thereof, the third T-cell subpopulation is specific to NS5, or a functional fragment thereof, the fourth T-cell subpopulation is specific to NS6, or a functional fragment thereof, and the fifth T-cell subpopulation is specific to any one of NS7, VP1 and VP2, or a functional fragment thereof. In some embodiments, the disclosed composition comprises at least five T-cell subpopulations, wherein the first T-cell subpopulation is specific to NS1-2, or a functional fragment thereof, the second T-cell subpopulation is specific to NS4, or a functional fragment thereof, the third T-cell subpopulation is specific to NS5, or a functional fragment thereof, the fourth T-cell subpopulation is specific to NS7, or a functional fragment thereof, and the fifth T-cell subpopulation is specific to any one of VP1 and VP2, or a functional fragment thereof. In some embodiments, the disclosed composition comprises at least five T-cell subpopulations, wherein the first T-cell subpopulation is specific to NS1-2, or a functional fragment thereof, the second T-cell subpopulation is specific to NS4, or a functional fragment thereof, the third T-cell subpopulation is specific to NS5, or a functional fragment thereof, the fourth T-cell subpopulation is specific to VP1, or a functional fragment thereof, and the fifth T-cell subpopulation is specific to VP2, or a functional fragment thereof. In some embodiments, the disclosed composition comprises at least five T-cell subpopulations, wherein the first T-cell subpopulation is specific to NS1-2, or a functional fragment thereof, the second T-cell subpopulation is specific to NS4, or a functional fragment thereof, the third T-cell subpopulation is specific to NS6, or a functional fragment thereof, the fourth T-cell subpopulation is specific to NS7, or a functional fragment thereof, and the fifth T-cell subpopulation is specific to any one of VP1 and VP2, or a functional fragment thereof. In some embodiments, the disclosed composition comprises at least five T-cell subpopulations, wherein the first T-cell subpopulation is specific to NS1-2, or a functional fragment thereof, the second T-cell subpopulation is specific to NS4, or a functional fragment thereof, the third T-cell subpopulation is specific to NS6, or a functional fragment thereof, the fourth T-cell subpopulation is specific to VP1, or a functional fragment thereof, and the fifth T-cell subpopulation is specific to VP2, or a functional fragment thereof. In some embodiments, the disclosed composition comprises at least five T-cell subpopulations, wherein the first T-cell subpopulation is specific to NS1-2, or a functional fragment thereof, the second T-cell subpopulation is specific to NS4, or a functional fragment thereof, the third T-cell subpopulation is specific to NS7, or a functional fragment thereof, the fourth T-cell subpopulation is specific to VP1, or a functional fragment thereof, and the fifth T-cell subpopulation is specific to VP2, or a functional fragment thereof. In some embodiments, the disclosed composition comprises at least five T-cell subpopulations, wherein the first T-cell subpopulation is specific to NS1-2, or a functional fragment thereof, the second T-cell subpopulation is specific to NS5, or a functional fragment thereof, the third T-cell subpopulation is specific to NS6, or a functional fragment thereof, the fourth T-cell subpopulation is specific to NS7, or a functional fragment thereof, and the fifth T-cell subpopulation is specific to any one of VP1 and VP2, or a functional fragment thereof. In some embodiments, the disclosed composition comprises at least five T-cell subpopulations, wherein the first T-cell subpopulation is specific to NS1-2, or a functional fragment thereof, the second T-cell subpopulation is specific to NS5, or a functional fragment thereof, the third T-cell subpopulation is specific to NS6, or a functional fragment thereof, the fourth T-cell subpopulation is specific to VP1, or a functional fragment thereof, and the fifth T-cell subpopulation is specific to VP2, or a functional fragment thereof. In some embodiments, the disclosed composition comprises at least five T-cell subpopulations, wherein the first T-cell subpopulation is specific to NS1-2, or a functional fragment thereof, the second T-cell subpopulation is specific to NS6, or a functional fragment thereof, the third T-cell subpopulation is specific to NS7, or a functional fragment thereof, the fourth T-cell subpopulation is specific to VP1, or a functional fragment thereof, and the fifth T-cell subpopulation is specific to VP2, or a functional fragment thereof. In some embodiments, the disclosed composition comprises at least five T-cell subpopulations, wherein the first T-cell subpopulation is specific to NS3, or a functional fragment thereof, the second T-cell subpopulation is specific to NS4, or a functional fragment thereof, the third T-cell subpopulation is specific to NS5, or a functional fragment thereof, the fourth T-cell subpopulation is specific to NS6, or a functional fragment thereof, and the fifth T-cell subpopulation is specific to any one of NS7, VP1 and VP2, or a functional fragment thereof. In some embodiments, the disclosed composition comprises at least five T-cell subpopulations, wherein the first T-cell subpopulation is specific to NS3, or a functional fragment thereof, the second T-cell subpopulation is specific to NS4, or a functional fragment thereof, the third T-cell subpopulation is specific to NS5, or a functional fragment thereof, the fourth T-cell subpopulation is specific to NS7, or a functional fragment thereof, and the fifth T-cell subpopulation is specific to any one of VP1 and VP2, or a functional fragment thereof. In some embodiments, the disclosed composition comprises at least five T-cell subpopulations, wherein the first T-cell subpopulation is specific to NS3, or a functional fragment thereof, the second T-cell subpopulation is specific to NS4, or a functional fragment thereof, the third T-cell subpopulation is specific to NS5, or a functional fragment thereof, the fourth T-cell subpopulation is specific to VP1, or a functional fragment thereof, and the fifth T-cell subpopulation is specific to VP2, or a functional fragment thereof. In some embodiments, the disclosed composition comprises at least five T-cell subpopulations, wherein the first T-cell subpopulation is specific to NS3, or a functional fragment thereof, the second T-cell subpopulation is specific to NS4, or a functional fragment thereof, the third T-cell subpopulation is specific to NS6, or a functional fragment thereof, the fourth T-cell subpopulation is specific to NS7, or a functional fragment thereof, and the fifth T-cell subpopulation is specific to any one of VP1 and VP2, or a functional fragment thereof. In some embodiments, the disclosed composition comprises at least five T-cell subpopulations, wherein the first T-cell subpopulation is specific to NS3, or a functional fragment thereof, the second T-cell subpopulation is specific to NS4, or a functional fragment thereof, the third T-cell subpopulation is specific to NS6, or a functional fragment thereof, the fourth T-cell subpopulation is specific to VP1, or a functional fragment thereof, and the fifth T-cell subpopulation is specific to VP2, or a functional fragment thereof. In some embodiments, the disclosed composition comprises at least five T-cell subpopulations, wherein the first T-cell subpopulation is specific to NS3, or a functional fragment thereof, the second T-cell subpopulation is specific to NS4, or a functional fragment thereof, the third T-cell subpopulation is specific to NS7, or a functional fragment thereof, the fourth T-cell subpopulation is specific to VP1, or a functional fragment thereof, and the fifth T-cell subpopulation is specific to VP2, or a functional fragment thereof. In some embodiments, the disclosed composition comprises at least five T-cell subpopulations, wherein the first T-cell subpopulation is specific to NS3, or a functional fragment thereof, the second T-cell subpopulation is specific to NS5, or a functional fragment thereof, the third T-cell subpopulation is specific to NS6, or a functional fragment thereof, the fourth T-cell subpopulation is specific to NS7, or a functional fragment thereof, and the fifth T-cell subpopulation is specific to any one of VP1 and VP2, or a functional fragment thereof. In some embodiments, the disclosed composition comprises at least five T-cell subpopulations, wherein the first T-cell subpopulation is specific to NS3, or a functional fragment thereof, the second T-cell subpopulation is specific to NS5, or a functional fragment thereof, the third T-cell subpopulation is specific to NS6, or a functional fragment thereof, the fourth T-cell subpopulation is specific to VP1, or a functional fragment thereof, and the fifth T-cell subpopulation is specific to VP2, or a functional fragment thereof. In some embodiments, the disclosed composition comprises at least five T-cell subpopulations, wherein the first T-cell subpopulation is specific to NS3, or a functional fragment thereof, the second T-cell subpopulation is specific to NS6, or a functional fragment thereof, the third T-cell subpopulation is specific to NS7, or a functional fragment thereof, the fourth T-cell subpopulation is specific to VP1, or a functional fragment thereof, and the fifth T-cell subpopulation is specific to VP2, or a functional fragment thereof.

In some embodiments, the mononuclear cell sample from which the T-cell subpopulations are isolated is derived from the same human subject to which the composition is also administered (autologous). In some embodiments, the mononuclear cell sample from which the T-cell subpopulations are isolated is derived from a cell donor (allogeneic). In certain embodiments, the allogeneic T-cell subpopulation composition has at least one HLA allele or HLA allele combination in common with the patient. In certain embodiments, the allogeneic T-cell subpopulation composition has more than one HLA allele or HLA allele combination in common with the patient. In certain embodiments, the norovirus antigen activity of the disclosed composition is through at least one HLA allele or HLA allele combination in common with the patient. In certain embodiments, the allogeneic T-cell subpopulations comprising the disclosed composition are recognized through the same shared HLA restriction. In certain embodiments, the allogeneic T-cell subpopulations comprising the disclosed composition are recognized through different shared HLA restrictions.

In another aspect, the present disclosure provides a method of treating a disease or disorder comprising administering an effective amount of the T-cell composition disclosed herein to a patient, typically a human in need thereof.

In some embodiments, the method further comprises isolating a mononuclear cell sample from the patient, typically a human to which the disclosed composition is administered (autologous), wherein the disclosed composition comprises T-cell subpopulations made from the mononuclear cell sample.

In some embodiments, the method further comprises isolating a mononuclear cell sample from a cell donor (allogeneic), wherein the disclosed composition comprises T-cell subpopulations made from the mononuclear cell sample. In certain embodiments, the allogeneic disclosed composition has at least one HLA allele or HLA allele combination in common with the patient. In certain embodiments, the allogeneic disclosed composition has more than one HLA allele or HLA allele combination in common with the patient. In certain embodiments, the norovirus antigen activity of the disclosed composition is through at least one HLA allele or HLA allele combination in common with the patient. In certain embodiments, the norovirus antigen activity of the disclosed composition is through more than one HLA allele or HLA allele combination in common with the patient. In certain embodiments, the allogeneic T-cell subpopulations comprising the disclosed composition are recognized through the same shared HLA restriction. In certain embodiments, the allogeneic T-cell subpopulations comprising the disclosed composition are recognized through different shared HLA restrictions. In certain embodiments, the disclosed composition selected has the most shared HLA alleles or allele combinations and the highest norovirus antigen specificity.

In certain embodiments, the method further comprises selecting the disclosed composition based on the levels of circulating norovirus-specific T-cells present in the patient after administration of a disclosed composition. Methods of measuring the levels of circulating norovirus-specific T-cells present in the patient are known in the art and non-limiting exemplary methods include Elispot assay, TCR sequencing, intracellular cytokine staining, and through the uses of MHC-peptide multimers.

### Method of Treating a Patient by Administering a Disclosed Composition

In another aspect, the present disclosure provides a method of treating a subject having virus infection by administering one of the disclosed compositions. In some embodiments, the patient is infected by norovirus and at least one of cytomegalovirus (CMV), Epstein-Barr virus (EBV), Adenovirus, human herpesvirus 6 (HHV-6). In some embodiments, the patient is infected by norovirus. In some embodiments, the present disclosure includes a method to treat a patient with an acute or chronic norovirus infection, typically a human, by administering an effective amount of a disclosed composition described herein.

The dose administered may vary. In some embodiments, the disclosed composition is administered to a patient, such as a human, in a dose ranging from about 1×10⁶ cells/m² to about 1×10⁸ cells/m². The dose can be a single dose, for example, comprising the combination of all of the T-cell subpopulations comprising the disclosed composition, or multiple separate doses, wherein each dose comprises a separate T-cell subpopulation and the collective separate doses of T-cell subpopulations comprise the total disclosed composition. In some embodiments, the disclosed composition dosage is about 1×10⁶ cells/m², about 2×10⁶ cells/m², about 3×10⁶ cells/m², about 4×10⁶ cells/m², about 5×10⁶ cells/m², about 6×10⁶ cells/m², about 7×10⁶ cells/m², about 8×10⁶ cells/m², about 9×10⁶ cells/m², about 1×10′ cells/m², about 2×cells/m², about 3×10′ cells/m², about 4×cells/m², about 5×10′ cells/m², about 6×cells/m², about 7×10′ cells/m², about 8×cells/m², about 9×cells/m², or about 1×10⁸ cell s/m².

The disclosed composition may be administered by any suitable method. In some embodiments, the disclosed composition is administered to a subject, such as a human, as an infusion, and in a particular embodiment, an infusion with a total volume of about 1 to about 10 cc. In some embodiments, the disclosed composition is administered to a subject as 1, 2, 3, 4, 5, 6, 7, 8, 9, or 10 cc infusion. In some embodiments, the disclosed composition when present as an infusion is administered to a subject over about 10, 20, 30, 40, 50, 60 or more minutes to the subject in need thereof.

In some embodiments, a subject receiving an infusion has vital signs monitored before, during, and about 1-hour post infusion of the disclosed composition. In certain embodiments, patients with stable disease (SD), partial response (PR), or complete response (CR) up to about 6 weeks after initial infusion may be eligible to receive additional infusions, for example, 1, 2, 3, 4, 5, 6, 7, 8, 9 or 10 additional infusions several weeks apart, for example, up to about 2, 3, 4, 5, 6, 7, 8, 9 or 10 weeks apart.

In some embodiments the TCR comprises a sequence comprising at least about 75%, 80%, 85%, 90%, 91%, 92%, 93%, 94%, 95%, 96%, 97%, 98%, 99% or 100% to:

RMSIGLLCCAALSLLWAGPVNAGVTGTPKFGVLKTGGSMTLGCAGDMNH
EYMSWYRGDPGMGLRLIHYSVGAGITDGGEVPNGYNVSRSTTEDFPLRL
LSAAPSGTSVYFCASSYVGNTGELFFGEGSRLTVLEDLKNVFPPEVAVF
EPSEAEISHTCKATLVCLATGFYPDHVELSWWVNGKEVHSGVSTDPGPL
KEGPALNDSRYCLSSRLRVSATFWGNPRNHFRCGVGFYGLSENDEWTOD
RAKPVTOIVSAEAWGRADCGFTSESYOOGVLSATILYEILLGKATLYAV
LVSALVLMAMVKRKDSRG (b chain)
or
METLLGLLILWLGLGWVSSKGEVTGIPAALSVPEGENLVLNCSFTDSAI
YNLGWFRGDPGKGLTSLLLIGSSGREGTSGRLNASLDKSSGRSTLYIAA
SGPGDSATYLCAVRPLYGGSYIPTFGRGTSLIVHPYIONPDPAVYOLRD
SKSSDKSVCLFTDFDSOTNVSOSKDSDVYITDKTVLDMRSMDFKSNSAV
AWSNKSDFACANAFNNSIIPEDTFFPSPESSCDVKLVEKSFETDTNLNF
CNLSVIGFRILLLKVAGFNLLMTLRLWSS (a chain).

In another embodiment, the TCR in the nucleic acid construct is affinity matured.

In another embodiment, the TCR comprises an a chain and/or a b chain. In another embodiment, the TCR is a NY-ESO-1 TCR. In another embodiment, the nucleic acid sequence of the NY-ESO-1 TCR comprises a nucleic acid sequence having at least 80%, 85%, 90%, 95%, 96%, 97%, 98%, 99%, or 100% sequence identity to TCR a chain. In another embodiment, the nucleic acid sequence of the NY-ESO-1 TCR comprises a nucleic acid sequence having at least 80%, 85%, 90%, 95%, 96%, 97%, 98%, 99%, or 100% sequence identity to TCR b chain. In some embodiments, the nucleic acid sequence of the NY-ESO-1 TCR a chain comprises a nucleic acid sequence having at least 80%, 85%, 90%, 95%, 96%, 97%, 98%, 99%, or 100% sequence identity to both a and b chains.

### Administration of Disclosed Compositions

Methods for administration of cells for adoptive cell therapy are known and may be used in connection with the provided methods and disclosed compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

The administration of the disclosed composition may vary. In one aspect, the disclosed composition may be administered to a patient such as a human at an interval selected from about once every 1 week, 2 weeks, 3 weeks, 4 weeks, 5 weeks, 6 weeks, 7 weeks, 8 weeks, or more after the initial administration of the disclosed composition. In a typical embodiment, the disclosed composition is administered in an initial dose then at every 4 weeks thereafter. In some embodiments, the disclosed composition may be administered repetitively to 1, 2, 3, 4, 5, 6, or more times after the initial administration of the composition. In a typical embodiment, the disclosed composition is administered repetitively up to 10 more times after the initial administration of the disclosed composition. In an alternative embodiment, the disclosed composition is administered more than 10 times after the initial administration of the disclosed composition.

The disclosure relates to pharmaceutical compositions comprising therapeutically effective amounts of disclosed T-cells and a pharmaceutically acceptable carrier. In some embodiments, the disclosed compositions are administered to a subject in the form of a pharmaceutical composition, such as a composition comprising the cells or cell populations and a pharmaceutically acceptable carrier or excipient. The pharmaceutical compositions in some embodiments additionally comprise other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc., or immunosuppressive agents, e.g., cyclosporin A, tacrolimus, mycophenolate, rapamycin, corticosteroids, etc. In some embodiments, the agents are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The choice of carrier in the pharmaceutical composition may be determined in part by the particular method used to administer the cell composition. Accordingly, there is a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition.

In addition, buffering agents in some aspects are included in the composition. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins 21st ed. (May 1, 2005).

In some embodiments, the pharmaceutical composition comprises the disclosed composition in an amount that is effective to treat or prevent the disease or condition, such as a therapeutically effective or prophylactically effective amount. Thus, in some embodiments, the methods of administration include administration of the disclosed composition at effective amounts. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, the disclosed composition is administered at a desired dosage, which in some aspects includes a desired dose or number of cells and/or a desired ratio of T-cell subpopulations. Thus, the dosage of cells in some embodiments is based on a total number of cells (or number per m² body surface area or per kg body weight) and a desired ratio of the individual populations or sub-types. In some embodiments, the dosage of cells is based on a desired total number (or number per m² body surface area or per kg of body weight) of cells in the individual populations or of individual cell types. In some embodiments, the dosage is based on a combination of such features, such as a desired number of total cells, desired ratio, and desired total number of cells in the individual populations.

In some embodiments, the disclosed composition is administered at or within a tolerated difference of a desired dose of total cells, such as a desired dose of T cells. In some aspects, the desired dose is a desired number of cells, a desired number of cells per unit of body surface area or a desired number of cells per unit of body weight of the subject to whom the cells are administered, e.g., cells/m² or cells/kg. In some aspects, the desired dose is at or above a minimum number of cells or minimum number of cells per unit of body surface area or body weight. In some aspects, among the total cells, administered at the desired dose, the individual populations or sub-types are present at or near a desired output ratio as described herein, e.g., within a certain tolerated difference or error of such a ratio.

In some embodiments, the cells are administered at or within a tolerated difference of a desired dose. In some aspects, the desired dose is a desired number of cells, or a desired number of such cells per unit of body surface area or body weight of the subject to whom the cells are administered, e.g., cells/m² or cells/kg. In some aspects, the desired dose is at or above a minimum number of cells of the population, or minimum number of cells of the population per unit of body surface area or body weight.

Thus, in some embodiments, the dosage is based on a desired fixed dose of total cells and a desired ratio, and/or based on a desired fixed dose of two or more, e.g., each, of the individual T-cell subpopulations. Thus, in some embodiments, the dosage is based on a desired fixed or minimum dose of T-cell subpopulations and a desired ratio thereof.

In certain embodiments, the disclosed composition is administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual T-cell subpopulations of cells is within a range of between at or about 10⁴ and at or about 10⁹ cells/meter² (m²) body surface area, such as between 10⁵ and 10⁶ cells/m² body surface area, for example, at or about 1×10⁵ cells/m², 1.5×10⁵ cells/m², 2×10⁵ cells/m², or 1×10⁶ cells/m² body surface area. For example, in some embodiments, the cells are administered at, or within a certain range of error of, between at or about 10⁴ and at or about 10⁹ T cells/meter² (m²) body surface area, such as between 10⁵ and 10⁶ T cells/m² body surface area, for example, at or about 1×10⁵ T cells/m², 1.5×10⁵ T cells/m², 2×10⁵ T cells/m², or 1×10⁶ T cells/m² body surface area.

In some embodiments, the cells are administered at or within a certain range of error of between at or about 10⁴ and at or about 10⁹ cells/meter² (m²) body weight, such as between 10⁵ and 10⁶ cells/m²body weight, for example, at or about 1×10⁵ cells/m², 1.5×10⁵ cells/m², 2×10⁵ cells/kg, or 1×10⁶ cells/m² body surface area. In some embodiments, the cells are administered at or within a certain range of error of between at or about 10′ and at or about 5×cells/m² body weight.

Product Release Testing and Characterization of T-cell subpopulations

Prior to infusion, the disclosed composition may be characterized for safety and release testing. Product release testing, also known as lot or batch release testing, is an important step in the quality control process of drug substances and drug products. This testing verifies that a T-cell subpopulation and/or disclosed composition meets a pre-determined set of specifications. Pre-determined release specifications for T-cell subpopulations and disclosed compositions include confirmation that the cell product is >70% viable, has <5.0 EU/ml of endotoxin, is negative for aerobic, anaerobic, fungal pathogens and mycoplasma, and lacks reactivity to allogeneic PHA blasts, for example, with less than about 10% lysis to PHA blasts. The phenotype of the disclosed composition may be determined with requirements for clearance to contain, in one non-limiting embodiment, <2% dendritic cells and <2% B cells. The HLA identity between the disclosed composition and the donor is also confirmed.

Antigen specificity of the T-cell subpopulations can be tested via an Interferon-γ Enzyme-Linked Immunospot (IFN-γ ELISpot) assay. Other cytokines can also be utilized to measure antigen specificity, including but not limited to, TNFα and IL-4. Pre-stimulated effector cells and target cells pulsed with the norovirus antigen of interest are incubated in a 96-well plate (pre-incubated with anti-INFγ antibody) at an E/T ratio of about 1:2. They are compared with no-norovirus antigen control, an irrelevant peptide not used for T-cell generation, and SEB as a positive control. After washing, the plates are incubated with a biotinylated anti-IFNγ antibody. Spots are detected by incubating with streptavidin-coupled alkaline phosphatase and substrate. Spot forming cells (SFCs) are counted and evaluated using an automated plate reader.

The phenotype of the disclosed composition can be determined by extracellular antibody staining with anti-CD3, CD4, CD8, CD45, CD19, CD16, CD56, CD14, CD45, CD83, HLA-DR, TCRαβ, TCRγδ and analyzed on a flow cytometer. Annexin-V and PI antibodies can be used as viability controls, and data analyzed with FlowJo Flow Cytometry software (Treestar, Ashland, Oreg., USA).

The lytic capacity of T-cell subpopulations may be evaluated via ⁵¹Chromium (⁵¹Cr) and Europium (Eu)-release cytotoxicity assays to test recognition and lysis of target cells pulsed with viral peptides by the T-cell subpopulations and disclosed compositions.

Typically, activated primed T-cells (effector cells) may be tested against ⁵¹Cr-labeled target cells at effector-to-target ratios of, for example, about 40:1, about 20:1, about 10:1, and about 5:1. Cytolytic activity can be determined by measuring ⁵¹Cr release into the supernatant on a gamma-counter. Spontaneous release is assessed by incubating target cells alone, and maximum lysis by adding 1% Triton X-100. Specific lysis was calculated as: specific lysis (%)=(experimental release−spontaneous release)/(maximum release−spontaneous release)×100.

Europium-release assays may also be utilized to measure the lytic capacity of T-cell subpopulations and disclosed compositions. This is a non-radioactive alternative to the conventional Chromium-51 (⁵¹Cr) release assay and works on the same principle as the radioactive assay. Target cells are first loaded with an acetoxymethyl ester of BATDA. The ligand penetrates the cell membrane quickly. Within the cell, the ester bonds are hydrolyzed to form a hydrophilic ligand (TDA), which no longer passes through the cell membrane. If cells are lysed by an effector cell, TDA is released outside the cell into the supernatant. Upon addition of Europium solution to the supernatant, Europium can form a highly fluorescent and stable chelate with the released TDA (EuTDA). The measured fluorescence signal correlates directly with the number of lysed cells in the cytotoxicity assay. Specific lysis was calculated as: specific lysis (%)=(experimental release−spontaneous release)/(maximum release−spontaneous release)×100.

### Monitoring

Following administration of the cells, the biological activity of the administered cell populations in some embodiments is measured, e.g., by any of a number of known methods. Parameters to assess include specific binding of a T-cell or other immune cell to antigen, in vivo, e.g., by imaging, or ex vivo, e.g., by ELISA or flow cytometry. In certain embodiments, the ability of the administered cells to destroy target cells may be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004), all incorporated herein by reference. In certain embodiments, the biological activity of the cells is measured by assaying expression and/or secretion of one or more cytokines, such as IFNγ, IL-2, and TNF. In some aspects, the biological activity is measured by assessing clinical outcome, such as reduction in disease symptoms or viral load.

### Combination Therapies

In one aspect of the disclosure, the T-cell compositions disclosed herein can be beneficially administered in combination with another therapeutic regimen for beneficial, additive, or synergistic effects. In such combination therapies, the disclosed composition provides a norovirus therapy to clear the norovirus infecting the patient.

In some embodiments, the disclosed composition is administered in combination with another therapy to treat an underlying hematological malignancy. In some embodiments, the disclosed composition is administered in combination with another therapy to treat an underlying solid tumor. The second therapy can be a pharmaceutical or a biologic agent (for example an antibody) to increase the efficacy of treatment with a combined or synergistic approach.

In some embodiments, the additional therapy is a monoclonal antibody (MAb). Some MAbs stimulate an immune response that destroys tumor cells. Similar to the antibodies produced naturally by B cells, these MAbs “coat” the tumor cell surface, triggering its destruction by the immune system. FDA-approved MAbs of this type include rituximab, which targets the CD20 antigen found on non-Hodgkin lymphoma cells, and alemtuzumab, which targets the CD52 antigen found on B-cell chronic lymphocytic leukemia (CLL) cells. Rituximab may also trigger cell death (apoptosis) directly. Another group of MAbs stimulates an antitumor immune response by binding to receptors on the surface of immune cells and inhibiting signals that prevent immune cells from attacking the body's own tissues, including tumor cells. Other MAbs interfere with the action of proteins that are necessary for tumor growth. For example, bevacizumab targets vascular endothelial growth factor(VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels. Similarly, cetuximab and panitumumab target the epidermal growth factor receptor (EGFR). MAbs that bind to cell surface growth factor receptors prevent the targeted receptors from sending their normal growth-promoting signals. They may also trigger apoptosis and activate the immune system to destroy tumor cells. Another group of tumor therapeutic MAbs are the immunoconjugates. These MAbs, which are sometimes called immunotoxins or antibody-drug conjugates, consist of an antibody attached to a cell-killing substance, such as a plant or bacterial toxin, a chemotherapy drug, or a radioactive molecule. The antibody latches onto its specific antigen on the surface of a tumor cell, and the cell-killing substance is taken up by the cell. FDA-approved conjugated MAbs that work this way include 90Y-ibritumomab tiuxetan, which targets the CD20 antigen to deliver radioactive yttrium-90 to B-cell non-Hodgkin lymphoma cells; ¹³¹I-tositumomab, which targets the CD20 antigen to deliver radioactive ¹³¹I to non-Hodgkin lymphoma cells.

In some embodiments, the additional agent is an immune checkpoint inhibitor (ICI), for example, but not limited to PD-1 inhibitors, PD-L1 inhibitors, PD-L2 inhibitors, CTLA-4 inhibitors, LAG-3 inhibitors, TIM-3 inhibitors, and V-domain Ig suppressor of T-cell activation (VISTA) inhibitors, or combinations thereof.

In some embodiments, the immune checkpoint inhibitor is a PD-1 inhibitor that blocks the interaction of PD-1 and PD-L1 by binding to the PD-1 receptor, and in turn inhibits immune suppression. In some embodiments, the immune checkpoint inhibitor is a PD-1 immune checkpoint inhibitor selected from nivolumab (Opdivo®), pembrolizumab (Keytruda®), pidilizumab, AMP-224 (AstraZeneca and MedImmune), PF-06801591 (Pfizer), MEDI0680 (AstraZeneca), PDR001 (Novartis), REGN2810 (Regeneron), MGA012 (MacroGenics), BGB-A3 17 (BeiGene) SHR-12-1 (Jiangsu Hengrui Medicine Company and Incyte Corporation), TSR-042 (Tesaro), and the PD-L1/VISTA inhibitor CA-170 (Curis Inc.).

In some embodiments, the immune checkpoint inhibitor is the PD-1 immune checkpoint inhibitor nivolumab (Opdivo®) administered in an effective amount for the treatment of Hodgkin's lymphoma. In another aspect of this embodiment, the immune checkpoint inhibitor is the PD-1 immune checkpoint inhibitor pembrolizumab (Keytruda®) administered in an effective amount. In an additional aspect of this embodiment, the immune checkpoint inhibitor is the PD-1 immune checkpoint inhibitor pidilizumab (Medivation) administered in an effective amount for refractory diffuse large B-cell lymphoma (DLBCL).

In some embodiments, the immune checkpoint inhibitor is a PD-L1 inhibitor that blocks the interaction of PD-1 and PD-L1 by binding to the PD-L1 receptor, and in turn inhibits immune suppression. PD-L1 inhibitors include, but are not limited to, atezolizumab, durvalumab, KNO35CA-170 (Curis Inc.), and LY3300054 (Eli Lilly).

In some embodiments, the immune checkpoint inhibitor is the PD-L1 immune checkpoint inhibitor atezolizumab (Tecentriq®) administered in an effective amount. In another aspect of this embodiment, the immune checkpoint inhibitor is durvalumab (AstraZeneca and MedImmune) administered in an effective. In yet another aspect of the embodiment, the immune checkpoint inhibitor is KN035 (Alphamab). An additional example of a PD-L1 immune checkpoint inhibitor is BMS-936559 (Bristol-Myers Squibb), although clinical trials with this inhibitor have been suspended as of 2015.

In one aspect of this embodiment, the immune checkpoint inhibitor is a CTLA-4 immune checkpoint inhibitor that binds to CTLA-4 and inhibits immune suppression. CTLA-4 inhibitors include, but are not limited to, ipilimumab, tremelimumab (AstraZeneca and MedImmune), AGEN1884 and AGEN2041 (Agenus).

In some embodiments, the CTLA-4 immune checkpoint inhibitor is ipilimumab (Yervoy®) administered in an effective amount

In another embodiment, the immune checkpoint inhibitor is a LAG-3 immune checkpoint inhibitor. Examples of LAG-3 immune checkpoint inhibitors include, but are not limited to, BMS-986016 (Bristol-Myers Squibb), GSK2831781 (GlaxoSmithKline), IMP321 (Prima BioMed), LAG525 (Novartis), and the dual PD-1 and LAG-3 inhibitor MGD013 (MacroGenics). In yet another aspect of this embodiment, the immune checkpoint inhibitor is a TIM-3 immune checkpoint inhibitor. A specific TIM-3 inhibitor includes, but is not limited to, TSR-022 (Tesaro). Other immune checkpoint inhibitors for use in combination with the disclosure described herein include, but are not limited to, B7-H3/CD276 immune checkpoint inhibitors such as MGA217, indoleamine 2,3-dioxygenase (IDO) immune checkpoint inhibitors such as Indoximod and INCB024360, killer immunoglobulin-like receptors (KIRs) immune checkpoint inhibitors such as Lirilumab (BMS-986015), carcinoembryonic antigen cell adhesion molecule (CEACAM) inhibitors (e.g., CEACAM-1, -3 and/or -5). Exemplary anti-CEACAM-1 antibodies are described in WO 2010/125571, WO 2013/082366 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In other embodiments, the anti-CEACAM antibody binds to CEACAM-5 as described in, e.g., Zheng et al. PLoS One. 2010 Sep. 2; 5(9). pii: e12529 (DOI:10: 1371/journal.pone.0021146), or cross-reacts with CEACAM-1 and CEACAM-5 as described in, e.g., WO 2013/054331 and US 2014/0271618. Still other checkpoint inhibitors can be molecules directed to B and T lymphocyte attenuator molecule (BTLA), for example as described in Zhang et al., Monoclonal antibodies to B and T lymphocyte attenuator (BTLA) have no effect on in vitro B cell proliferation and act to inhibit in vitro T cell proliferation when presented in a cis, but not trans, format relative to the activating stimulus, Clin Exp Immunol. 2011 January; 163(1): 77-87.

Current chemotherapeutic drugs that may be used in combination with the disclosed composition described herein include those used to treat AML including cytarabine (cytosine arabinoside or ara-C) and the anthracycline drugs (such as daunorubicin/daunomycin, idarubicin, and mitoxantrone). Some of the other chemo drugs that may be used to treat AML include: Cladribine (Leustatin®, 2-CdA), Fludarabine (Fludara®), Topotecan, Etoposide (VP-16), 6-thioguanine (6-TG), Hydroxyurea (Hydrea®), Corticosteroid drugs, such as prednisone or dexamethasone (Decadron®), Methotrexate (MTX), 6-mercaptopurine (6-MP), Azacitidine (Vidaza®), Decitabine (Dacogen®). Additional drugs include dasatinib and checkpoint inhibitors such as novolumab, Pembrolizumab, and atezolizumab.

Current chemotherapeutic drugs that may be used in combination with the Disclosed composition described herein include those used for CLL and other lymphomas including: purine analogs such as fludarabine (Fludara®), pentostatin (Nipent®), and cladribine (2-CdA, Leustatin®), and alkylating agents, which include chlorambucil (Leukeran®) and cyclophosphamide (Cytoxan®) and bendamustine (Treanda®). Other drugs sometimes used for CLL include doxorubicin (Adriamycin®), methotrexate, oxaliplatin, vincristine (Oncovin®), etoposide (VP-16), and cytarabine (ara-C). Other drugs include Rituximab (Rituxan), Obinutuzumab (Gazyva™), Ofatumumab (Arzerra®), Alemtuzumab (Campath®) and Ibrutinib (Imbruvica™).

Current chemotherapeutic drugs that may be used in combination with the Disclosed composition described herein include those used for CML including: Interferon, imatinib (Gleevec), the chemo drug hydroxyurea (Hydrea®), cytarabine (Ara-C), busulfan, cyclophosphamide (Cytoxan®), and vincristine (Oncovin®). Omacetaxine (Synribo®) is a chemo drug that was approved to treat CML that is resistant to some of the TKIs now in use.

Current chemotherapeutic drugs that may be used in combination with the Disclosed composition described herein include those used for CMML, for example, Deferasirox (Exjade®), cytarabine with idarubicin, cytarabine with topotecan, and cytarabine with fludarabine, Hydroxyurea (hydroxycarbamate, Hydrea®), azacytidine (Vidaza®) and decitabine (Dacogen®).

Current chemotherapeutic drugs that may be used in combination with the Disclosed composition described herein include those used for multiple myeloma include Pomalidomide (Pomalyst®), Carfilzomib (Kyprolis™), Everolimus (Afinitor®), dexamethasone (Decadron), prednisone and methylprednisolone (Solu-medrol®) and hydrocortisone.

Current chemotherapeutic drugs that may be used in combination with the Disclosed composition described herein include those used for Hodgkin's disease include Brentuximab vedotin (Adcetris™): anti-CD-30, Rituximab, Adriamycin® (doxorubicin), Bleomycin, Vinblastine, Dacarbazine (DTIC).

Current chemotherapeutic drugs that may be used in combination with the Disclosed composition described herein include those used for Non-Hodgkin's disease include Rituximab (Rituxan®), Ibritumomab (Zevalin®), tositumomab (Bexxar®), Alemtuzumab (Campath®) (CD52 antigen), Ofatumumab (Arzerra®), Brentuximab vedotin (Adcetris®) and Lenalidomide (Revlimid®).

Additional therapeutic agents that can be administered in combination with the disclosed compositions disclosed herein can include bevacizumab, sutinib, sorafenib, 2-methoxyestradiol, finasunate, vatalanib, vandetanib, aflibercept, volociximab, etaracizumab, cilengitide, erlotinib, cetuximab, panitumumab, gefitinib, trastuzumab, atacicept, rituximab, alemtuzumab, aldesleukine, atlizumab, tocilizumab, temsirolimus, everolimus, lucatumumab, dacetuzumab, atiprimod, natalizumab, bortezomib, carfilzomib, marizomib, tanespimycin, saquinavir mesylate, ritonavir, nelfinavir mesylate, indinavir sulfate, belinostat, panobinostat, mapatumumab, lexatumumab, oblimersen, plitidepsin, talmapimod, enzastaurin, tipifarnib, perifosine, imatinib, dasatinib, lenalidomide, thalidomide, simvastatin, and celecoxib.

In one aspect of the present disclosure, the T-cell compositions disclosed herein are administered in combination with at least one immunosuppressive agent. The immunosuppressive agent may be selected from the group consisting of a calcineurin inhibitor, e.g. a cyclosporin or an ascomycin, e.g. Cyclosporin A (NEORAL®), tacrolimus, a mTOR inhibitor, e.g. rapamycin or a derivative thereof, e.g. Sirolimus (RAPAMUNE®), Everolimus (Certican®), temsirolimus, biolimus-7, biolimus-9, a rapalog, e.g. azathioprine, campath 1H, a S1P receptor modulator, e.g. fingolimod or an analogue thereof, an anti-IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3®), Prednisone, ATGAM®, THYMOGLOBULIN®, Brequinar Sodium, 15-deoxyspergualin, tresperimus, Leflunomide ARAVA®, anti-CD25, anti-IL2R, Basiliximab (SIMULECT®), Daclizumab (ZENAPAX®), mizorbine, methotrexate, dexamethasone, pimecrolimus (Elidel®), abatacept, belatacept, etanercept (Enbrel®), adalimumab (Humira®), infliximab (Remicade®), an anti-LFA-1 antibody, natalizumab (Antegren®), Enlimomab, ABX-CBL, antithymocyte immunoglobulin, siplizumab, and efalizumab. Non-limiting examples of immunosuppressive agents that may be used in a combination therapy with the T-cell composition disclosed herein are provided in the table below.

| Medication | Typical dosage | Notes |
|---|---|---|
| Cyclosporin A | 3-5 mg/kg/day | |
| Tacrolimus | 0.15-0.4 mg/kg/day | |
| Mycophenolate | 500-1000 mg twice daily | |
| Rapamycin | 1-5 mg/day | |
| Corticosteroids | 0.25-2 mg/kg/day | Doses >0.5 mg/kg/day are |
| | (prednisone or | likely to inactivate VSTs |
| | equivalents) | |

In some aspects of the present disclosure, the disclosed composition described herein can be administered in combination with at least one anti-inflammatory agent. The anti-inflammatory agent can be a steroidal anti-inflammatory agent, a nonsteroidal anti-inflammatory agent, or a combination thereof. In some embodiments, anti-inflammatory drugs include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide di sodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof.

In one aspect of the present disclosure, the disclosed composition described herein can be administered in combination with at least one immunomodulatory agent.

In one aspect of the present disclosure, the disclosed composition described here can be administered in combination with at least one agent or drug that is currently used for treating the same viral infection. For example, the disclosed composition described herein can be administered in combination with at least one agent or drug that is currently used for treating CMV infection, e.g., valganciclovir, ganciclovir, cidofovir, foscarnet, etc. Likewise, when used to treat norovirus, the disclosed composition described herein can be administered in combination with at least one agent or drug that is currently used for treating norovirus infection, e.g., nitazoxnide, oral immunoglobulin, and favipiravir, with the typical dosage exemplified in the table below.

| Medication | Typical dosage | Notes |
|---|---|---|
| Nitazoxanide | 500 mg twice daily | |
| Oral immunoglobulin | 200-300 mg/kg/dose | |
| Favipiravir | 1200 mg twice daily | Investigational agent |

### Methods of Manufacturing Disclosed Compositions

T-cell subpopulations specific for a single norovirus antigen to be combined into the disclosed compositions for therapeutic administration described herein can be generated using any known method in the art or as described herein. Activated T-cell subpopulations that recognize at least one epitope of a norovirus antigen can be generated by any method known in the art or as described herein. Non-limiting exemplary methods of generating activated T-cell subpopulations that recognize at least one epitope of a norovirus antigen can be found in, for example Shafer et al., Leuk Lymphoma (2010) 51(5):870-880; Cruz et al., Clin Cancer Res., (2011) 17(22): 7058-7066; Quintarelli et al., Blood (2011) 117(12): 3353-3362; and Chapuis et al., Sci Transl Med (2013) 5(174):174ra27, all incorporated herein by reference.

Generally, generating the T-cell subpopulations of the disclosed compositions of the present disclosure may involve (i) collecting a peripheral blood mononuclear cell product from a donor; (ii) determining the HLA subtype of the mononuclear cell product; (iii) separating the monocytes and the lymphocytes of the mononuclear cell product; (iv) generating and maturing dendritic cells (DCs) from the monocytes; (v) pulsing the DCs with a norovirus antigen; (vi) optionally carrying out a CD45RA+ selection to isolate naïve lymphocytes; (vii) stimulating the naïve lymphocytes with the peptide-pulsed DCs in the presence of a cytokine cocktail; (viii) repeating the T-cell stimulation with fresh peptide-pulsed DCs or other peptide-pulsed antigen presenting cells in the presence of a cytokine cocktail; (ix) harvesting the T-cells and cryopreserving for future use.

In some aspects, generating the T-cell subpopulations of the disclosed compositions of the present disclosure may involve (i) collecting a peripheral blood mononuclear cell product from a donor; (ii) determining the HLA subtype of the mononuclear cell product; (iii) separating the monocytes and the lymphocytes of the mononuclear cell product; (iv) generating and maturing dendritic cells (DCs) from the monocytes; (v) pulsing the DCs with a norovirus antigen; (vi) optionally carrying out a CD45RA+ selection to isolate naïve T-cells; (vii) stimulating the naïve T-cells with the peptide-pulsed DCs in the presence of a cytokine cocktail; (viii) repeating the T-cell stimulation with fresh peptide-pulsed DCs or other peptide-pulsed antigen presenting cells in the presence of a cytokine cocktail; (ix) harvesting the T-cells and cryopreserving for future use.

In some embodiments, the T-cell subpopulation of the disclosed composition according to the present disclosure is primed and expanded for at least about 3, 5, 6, 7, 8, 9, 10, 11, 12, 13, 14, 15, 16, 17, 18, 19, or 20 days. In some embodiments, the T-cell subpopulation of the disclosed composition according to the present disclosure is primed and expanded for at least about 5-15 days. In some embodiments, the T-cell subpopulation of the disclosed composition according to the present disclosure is primed and expanded for at least about 10-12 days.

Collecting a Peripheral Blood Mononuclear Cell Product from a Donor

The generation of T-cell subpopulations to be specific to a single norovirus antigen generally requires a peripheral blood mononuclear cell (PBMC) product from a donor, either an allogeneic or autologous donor, as a starting material. Isolation of PBMCs is well known in the art. Non-limiting exemplary methods of isolating PBMCs are provided in Grievink, H. W., et al. (2016) “Comparison of three isolation techniques for human peripheral blood mononuclear cells: Cell recovery and viability, population composition, and cell functionality,” Biopreservation and BioBanking, which is incorporated herein by reference. The PBMC product can be isolated from whole blood, an apheresis sample, a leukapheresis sample, or a bone marrow sample provided by a donor. In some embodiments, the starting material is an apheresis sample, which provides a large number of initially starting mononuclear cells, potentially allowing a large number of different T-cell subpopulations to be generated. In some embodiments, the PBMC product is isolated from a sample containing peripheral blood mononuclear cells (PBMCs) provided by a donor. In some embodiments, the donor is a healthy donor. In some embodiments, the PBMC product is derived from cord blood. In some embodiments, the donor is the same donor providing stem cells for a hematopoietic stem cell transplant (HSCT).

### Determining HLA Subtype

When the T-cell subpopulations are generated from an allogeneic, healthy donor, the HLA subtype profile of the donor source is determined and characterized. Determining HLA subtype (i.e., typing the HLA loci) can be performed by any method known in the art. Non-limiting exemplary methods for determining HLA subtype can be found in Lange, V., et al., BMC Genomics (2014)15: 63; Erlich, H., Tissue Antigens (2012) 80:1-11; Bontadini, A., Methods (2012) 56:471-476; Dunn, P. P., Int J Immunogenet (2011) 38:463-473; and Hurley, C. K., “DNA-based typing of HLA for transplantation.” in Leffell, M. S., et al., eds., Handbook of Human Immunology, 1997. Boca Raton: CRC Press, each independently incorporated herein by reference. Preferably, the HLA-subtyping of each donor source is as complete as possible.

In some embodiments, the determined HLA subtypes include at least 4 HLA loci, preferably HLA-A, HLA-B, HLA-C, and HLA-DRB1. In some embodiments, the determined HLA subtypes include at least 6 HLA loci. In some embodiments, the determined HLA subtypes include at least 6 HLA loci. In some embodiments, the determined HLA subtypes include all of the known HLA loci. In general, typing more HLA loci is preferable for practicing the disclosure, since the more HLA loci that are typed, the more likely the allogeneic T-cell subpopulations selected will have highest activity relative to other allogeneic T-cell subpopulations that have HLA alleles or HLA allele combinations in common with the patient or the diseased cells in the patient.

### Separating the Monocytes and the Lymphocytes of the Peripheral Blood Mononuclear Cell Product

In general, the PBMC product may be separated into various cell-types, for example, into platelets, red blood cells, lymphocytes, and monocytes, and the lymphocytes and monocytes retained for initial generation of the T-cell subpopulations. The separation of PBMCs is known in the art. Non-limiting exemplary methods of separating monocytes and lymphocytes include Vissers et al., J Immunol Methods. 1988 Jun. 13; 110(2):203-7 and Wahl et al., Current Protocols in Immunology (2005) 7.6A.1-7.6A.10, which are incorporated herein by reference. For example, the separation of the monocytes can occur by plate adherence, by CD14+ selection, or other known methods. The monocyte fraction is generally retained in order to generate dendritic cells used as an antigen presenting cell in the T-cell subpopulation manufacture. The lymphocyte fraction of the PBMC product can be cryopreserved until needed, for example, aliquots of the lymphocyte fraction (˜5×10⁷ cells) can be cryopreserved separately for both Phytohemagglutinin (PHA) Blast expansion and T-cell subpopulation generation.

### Generating Dendritic Cells

The generation of mature dendritic cells used for antigen presentation to prime T-cells is well known in the art. Non-limiting exemplary methods are included in Nair et al., “Isolation and generation of human dendritic cells.” Current protocols in immunology (2012) 0 7: Unit7.32. doi:10.1002/0471142735.im0732s99 and Castiello et al., Cancer Immunol Immunother, 2011 April; 60(4):457-66, which are incorporated herein by reference. For example, the monocyte fraction can be plated into a closed system bioreactor such as the Quantum Cell Expansion System, and the cells allowed to adhere for about 2-4 hours at which point 1,000 U/mL of IL-4 and 800 U/mL GM-CSF can be added. The concentration of GM-C SF and IL-4 can be maintained. The dendritic cells can be matured using a cytokine cocktail. In some embodiments the cytokine cocktail consists of LPS (30 ng/mL), IL-4 (1,000 U/mL), GM-CSF (800 U/mL), TNF-Alpha (10 ng/mL), IL-6 (100 ng/mL), and IL-1beta (10 ng/mL). The dendritic cell maturation generally occurs in 2 to 5 days. In some embodiments, the adherent DCs are harvested and counted using a hemocytometer. In some embodiments, a portion of the DCs are cryopreserved for additional further stimulations.

### Pulsing the Dendritic Cells

The non-mature and mature dendritic cells are pulsed with one or more peptides, of a single norovirus antigen.

In some embodiments, the norovirus antigenic peptides used to pulse the non-mature and mature dendritic cells are from a pool or library of overlapping peptide fragments of the norovirus antigen, as provided for example, in commercially available overlapping peptide libraries. In some embodiments, the pool or library of overlapping peptide fragments of the norovirus antigen comprises peptide fragments of from about 10 to about 20 amino acids in length, for example, 15mers peptide fragments containing 9, 10 or 11 amino acids of overlap between each sequence formed. In some embodiments, the norovirus peptides used to pulse the non-mature and mature dendritic cells are from specifically selected HLA-restricted peptides generated by determining the HLA profile of the donor source, and including peptide epitopes derived from the targeted norovirus peptide that are active through the donor's HLA type. Methods of pulsing a dendritic cell with a norovirus peptides are known. For example, about 100 ng of one or more norovirus peptides, for example a peptide library, can be added per 10 million dendritic cells and incubated for about 30 to 120 minutes.

### Naïve T-cell Selection of Lymphocytes

In order to increase the potential number of specific norovirus antigen activated T-cells and reduce T-cells that target other antigens, it is preferable to utilize naïve T-cells as a starting material. To isolate naïve T-cells, the lymphocytes can undergo a selection, for example, CD45RA+ cells selection. CD45RA+ cell selection methods are generally known in the art. Non-limiting exemplary methods are found in Richards et al., Immune memory in CD4+ CD45RA+ T cells. Immunology. 1997; 91(3):331-339 and McBreen et al., J Virol. 2001 May; 75(9): 4091-4102, which are incorporated herein by reference. For example, to select for CD45RA⁺ cells, the cells can be labeled using 1 vial of CD45RA microbeads from Miltenyi Biotec per 1×10¹¹ cells after 5-30 minutes of incubation with 100 mL of CliniMACS buffer and approximately 3 mL of 10% human IVIG, 10 ug/mL DNAase I, and 200 mg/mL of magnesium chloride. After 30 minutes, cells will be washed sufficiently and resuspended in 20 mL of CliniMACS buffer. The bag will then be set up on the CLINIMACS Plus device and the selection program can be run according to manufacturer's recommendations. After the program is completed, cells can be counted, washed and resuspended in “CTL Media” consisting of 44.5% EHAA Click's, 44.5% Advanced RPMI, 10% Human Serum, and 1% GlutaMAX.

Stimulating Naïve T cells with Peptide-Pulsed Dendritic Cells

Prior to stimulating naïve T-cells with the dendritic cells, it may be preferable to irradiate the DCs, for example, at 25 Gy. The DCs and naïve T-cells are then co-cultured. The naïve T-cells can be co-cultured in a ratio range of DCs to T-cells of about 1:5-1:50, for example, about 1:5; about 1:10, about 1:15, about 1:20, about 1:25, about 1:30, about 1:35, about 1:40, about 1:45, or about 1:50. The DCs and T-cells are generally co-cultured with cytokines. In some embodiments, the cytokines are selected from a group consisting of IL-6 (100 ng/mL), IL-7 (10 ng/mL), IL-15 (5 ng/mL), IL-12 (10 ng/mL), and IL-21 (10 ng/mL).

### Second T-Cell Stimulation

In general, it is preferable to further stimulate the T-cell subpopulations with one or additional stimulation procedures. The additional stimulation can be performed with, for example, fresh DCs pulsed with the same peptides as used in the first stimulation, similarly to as described above. In some embodiments, the cytokines used during the second stimulation are selected from a group consisting of IL-7 (10 ng/mL) and IL-2 (100 U/mL).

Alternatively, peptide-pulsed PHA blasts can be used as the antigen presenting cell. The use of peptide-pulsed PHA blasts to stimulate and expand T-cells are well known in the art. Non-limiting exemplary methods can be found in Weber et al., Clin Cancer Res. 2013 Sep. 15; 19(18): 5079-5091 and Ngo et al., J. Immunother. 2014 May; 37(4): 193-203, which are incorporated herein by reference. The peptide-pulsed PHA blasts can be used to expand the T-cell subpopulation in a ratio range of PHA blasts to expanded T cells of about 10:1-1:10. For example, the ratio of PHA blasts to T-cells can be about 10:1, between about 10:1 and about 9:1, between about 9:1 and about 8:1, between about 8:1 and about 7:1, between about 7:1 and about 6:1, between about 6:1 and about 5:1, between about 5:1 and about 4:1, between about 4:1 and about 3:1, between about 3:1 and about 2:1, between about 2:1 and about 1:1, between about 1:1 and about 1:2, between about 1:2 and about 1:3, between about 1:3 and about 1:4, between about 1:4 and about 1:5, between about 1:5 and about 1:6, between about 1:6 and about 1:7, between about 1:7 and about 1:8, between about 1:8 and about 1:9, between about 1:9 and about 1:10. In general, cytokines are included in the co-culture, and are selected from the group consisting of IL-7 (10 ng/mL) and IL-2 (100 U/mL).

### Additional T-Cell Expansion and T-Cell Subpopulation Harvest

Additional T-cell stimulations may be necessary to generate the necessary number of T-cell subpopulations for use in the disclosed composition. Following any stimulation and expansion, the T-cell subpopulations are harvested, washed, and concentrated. In some embodiments, a solution containing a final concentration of 10% dimethyl sulfoxide (DMSO), 50% human serum albumin (HSA), and 40% Hank's Balanced Salt Solution (HBSS) will then be added to the cryopreservation bag. In some embodiments, the T-cell subpopulation will be cryopreserved in liquid nitrogen.

### Further Characterization of the T-cell Subpopulation

The T-cell subpopulations for use in the disclosed composition of the present disclosure are HLA-typed and can be further characterized prior to use or inclusion in the disclosed composition. For example, each of the T-cell subpopulations may be further characterized by, for example, one or more of i) determining the norovirus specificity of the T-cell subpopulation; ii) identifying the norovirus antigen epitope(s) the T-cell subpopulation is specific to; iii) determining whether the T-cell subpopulation includes MHC Class I or Class II restricted subsets or a combination of both; iv) correlating antigenic activity through the T-cell's corresponding HLA-allele; and v) characterizing the T-cell subpopulation's immune effector subtype concentration, for example, the population of effector memory cells, central memory cells, γδ T-cells, CD8+, CD4+, NKT-cell.

### Determining the Norovirus Antigen Specificity of the T-Cell Subpopulation

The T-cell subpopulations of the disclosed composition can be further characterized by determining each T-cell subpopulation's specificity for its targeted norovirus antigen. Specificity can be determined using any known procedure, for example, an ELISA based immunospot assay (ELISpot). In some embodiments, norovirus antigen specificity of the T-cell subpopulation is determined by ELISpot assay. ELISpot assays are widely used to monitor adaptive immune responses in both humans and animals. The method was originally developed from the standard ELISA assay to measure antibody secretion from B cells (Czerkinsky C. et al. (1983) A solid-phase enzyme-linked immunospot (ELISPOT) assay for enumeration of specific antibody-secreting cells. J. Immunol Methods 65: 109-21), which is incorporated herein by reference. The assay has since been adapted to detect secreted cytokines from T cells, for example IFN-γ, and is an essential tool for understanding the helper T cell response.

A T-cell ELISpot assay generally comprises the following steps:

i) a capture antibody specific for the chosen analyte, for example IFN-γ, is coated onto a PVDF plate;

ii) the plate is blocked, usually with a serum;

iii) the T-cell subpopulation is added along with the specific, targeted norovirus antigen;

iv) plates are incubated and secreted cytokines, for example IFN-γ, are captured by the immobilized antibody on the PVDF surface;

v) after washing, a biotinylated detection antibody is added to allow detection of the captured cytokine; and

vi) the secreted cytokine is visualized using an avidin-HRP or avidin-ALP conjugate and a colored precipitating substrate.

Each colored spot represents a cytokine secreting cell. The spots can be counted by eye or by using an automated plate-reader. Many different cytokines can be detected using this method including IL-2, IL-4, IL-17, IFN γ, TNFα, and granzyme B. The size of the spot is an indication of the per cell productivity and the avidity of the binding. The higher the avidity of the T cell recognition the higher the productivity resulting in large, well-defined spots.

### Identifying the Norovirus Epitope(s) the T-Cell Subpopulation is Specific to a Subject

The T-cell subpopulations of the disclosed composition can be further characterized by identifying the norovirus epitope(s) the T-cell subpopulation that is specific to a subject. General methods for identifying epitope(s) to which a T-cell population recognizes may include testing of overlapping peptide libraries encompassing norovirus antigens, in which the test utilized could include IFN-γ ELISpot, intracellular flow cytometry, or cytotoxicity assays via CD107a expression or 51-chromium release assay.

### Determining the T-cell Subpopulation's MHC-Class I or Class II Restricted Subsets

The T-cell subpopulations of the disclosed composition can be further characterized by determining the subpopulation's MHC Class I or Class II subset restriction response. This is done to determine whether epitope recognition is mediated by CD8+ (class I) or CD4+ (class II) T-cells. General methods for determining the MHC Class I or Class II response are generally known in the art. A non-limiting exemplary method is found in Weber et al., Clin Cancer Res. 2013 Sep. 15; 19(18): 5079-5091, which is incorporated herein by reference. For example, to determine HLA restriction response, T-cells can be pre-incubated with class I or II blocking antibodies for 1 hour before the addition of antigen peptides in an ELISPOT assay using autologous peptide-pulsed PHA blasts as targets with unpulsed PHA blasts as a control. IFNγ-secretion is measured in the presence of each blocking antibody. If, when pre-incubated with a class I blocking antibody, IFNγ-secretion is reduced to background levels then this is indicative of a class I restriction and the epitope recognition is mediated by CD8+ T cells. If, when pre-incubated with a class II blocking antibody, IFNγ-secretion is reduced to background levels then this is indicative of a class II restriction and the epitope recognition is mediated by CD4+ T cells.

The direct detection of antigen-specific T cells using tetramers of soluble peptide-major histocompatibilty complex (pMHC) molecules is widely used in both basic and clinical immunology. Tetrameric complexes of HLA molecules can be used to stain antigen-specific T cells in FACS analysis. In vitro synthesized soluble HLA-peptide complexes are used as tetrameric complexes to stain antigen specific T-cells in FACS analysis (Altman et al., Science 274: 94-96, 1996). T-cell subpopulations specific for norovirus antigens are stained with CD8 fluorescein isothiocyanate (FITC) and with phycoerythrin (PE)-labeled MHC pentamers at various timepoints during in vitro stimulation. Antigen specificity is measured by flow cytometry.

Correlating Antigenic Activity through the T-Cell's Corresponding HLA-Allele

The T-cell subpopulation can be further characterized by correlating antigenic activity through the T-cell subpopulation's corresponding HLA-allele. Correlating antigenic activity through the corresponding HLA-allele can be done using any known method. For example, in some embodiments, a HLA restriction assay is used to determine antigen activity through a corresponding allele. Methods to determine T-cell restriction are known in the art and involve inhibition with locus specific antibodies, followed by antigen presentation assays (ELISPOT) with panels of cell lines matched or mismatched at the various loci of interest (see, e.g., (Oseroff et al., J Immunol (2010) 185(2): 943-955; Oseroff et al., J. Immunol (2012) 189(2): 679-688; Wang Curr Protocols in immunol (2009) Chap. 20, page 10; Wilson et al., J. Virol. (2001) 75(9): 4195-4207), each independently incorporated herein by reference. Because epitope binding to HLA class II molecules is absolutely necessary (but not sufficient) for T cell activation, data from in vitro HLA binding assays has also been useful to narrow down the possible restrictions (Arlehamn et al., J Immunol (2012b) 188(10):5020-5031). This is usually accomplished by testing a given epitope for binding to the specific HLA molecules expressed in a specific donor and eliminating from further consideration HLA molecules to which the epitope does not bind. To determine the HLA restriction of the identified epitope, T cells can be plated in an IFN-γ ELISPOT assay with norovirus antigen peptide pulsed PHA blasts that match at a single allele, measuring the strongest antigen activity, and identifying the corresponding allele.

### Characterizing the T-cell Subpopulation's Immune Effector Subtype Concentration

The T-cell subpopulation is likely to be made up of different lymphocytic cell subsets, for example, a combination of CD4+ T-cells, CD8+ T-cells, CD3+/CD56+ Natural Killer T-cells (CD3+ NKT), and TCR γδ T-cells (γδ T-cells). In particular, the T-cell subpopulation likely include at least CD4+ T-cells and CD8+ T-cells that have been primed and are capable of targeting a single specific norovirus antigen for infected cell killing and/or cross presentation. The T-cell subpopulation may further comprise activated γδ T-cells and/or activated CD3+/CD56+ NKT cells capable of mediating anti-noroviral responses. Accordingly, the T-cell subpopulation may be further characterized by determining the population of various lymphocytic subtypes, and the further classification of such subtypes, for example, by determining the presence or absence of certain clusters of differentiation (CD) markers, or other cell surface markers, expressed by the cells and determinative of cell subtype.

In some embodiments, the T-cell subpopulation may be analyzed to determine CD8+ T-cell population, CD4+, T-cell population, γδ T-cell population, NKT-cell population, and other populations of lymphocytic subtypes. For example, the population of CD4+ T-cells within the T-cell subpopulation may be determined, and the CD4+ T-cell subtypes further determined. For example, the CD4+ T-cell population may be determined, and then further defined, for example, by identifying the population of T-helper 1 (Th₁), T-helper 2 (Th₂), T-helper 17 (Th₁₇), regulatory T cell (T_{reg}), follicular helper T-cell (T_{fh}), and T-helper 9 (Th₉). Likewise, the other lymphocytic subtypes comprising the T-cell subpopulation can be determined and further characterized.

In addition, the T-cell subpopulation can be further characterized, for example, for the presence, or lack thereof, of one or more markers associated with, for example, maturation or exhaustion. T cell exhaustion (Tₑₓ) is a state of dysfunction that results from persistent antigen and inflammation. Tₑₓ cell populations can be analyzed using multiple phenotypic parameters, either alone or in combination. Hallmarks commonly used to monitor T cell exhaustion are known in the art and include, but are not limited to, programmed cell death-1 (PD-1), CTLA-4/CD152 (Cytotoxic T-Lymphocyte Antigen 4), LAG-3 (Lymphocyte activation gene-3; CD223), TIM-3 (T cell immunoglobulin and mucin domain-3), 2B4/CD244/SLAMF4, CD160, and TIGIT (T cell Immunoreceptor with Ig and ITIM domains).

The T-cell subpopulations of the described compositions described herein can be subjected to further selection, if desired. For example, a particular T-cell subpopulation for inclusion in a disclosed composition described herein can undergo further selection through depletion or enriching for a sub-population. For example, following priming, expansion, and selection, the cells can be further selected for other cluster of differentiation (CD) markers, either positively or negatively. For example, following selection of for example CD4+ T-cells, the CD4+ T-cells can be further subjected to selection for, for example, a central memory T-cells (T_{cm}). For example, the enrichment for CD4+ T_{cm} cells comprises negative selection for cells expression a surface marker present on naïve T cells, such as CD45RA, or positive selection for cells expressing a surface marker present on T_{c}m cells and not present on naïve T-cells, for example CD45RO, CD62L, CCR7, CD27, CD127, and/or CD44. In addition, the T-cell subpopulations described herein can be further selected to eliminate cells expressing certain exhaustion markers, for example, programmed cell death-1 (PD-1), CTLA-4/CD152 (Cytotoxic T-Lymphocyte Antigen 4), LAG-3 (Lymphocyte activation gene-3; CD223), TIM-3 (T cell immunoglobulin and mucin domain-3), 2B4/CD244/SLAMF4, CD160, and TIGIT (T cell Immunoreceptor with Ig and ITIM domains).

Methods for characterizing lymphocytic cell subtypes are well known in the art, for example flow cytometry, which is described in Pockley et al., Curr Protoc Toxicol. 2015 Nov. 2; 66:18.8.1-34, which is incorporated herein by reference.

Identifying the disclosed composition Most Suitable for Administration

Characterization of each T-cell subpopulation composition allows for the selection of the most appropriate T-cell subpopulations for inclusion in the disclosed composition for any given patient. The goal is to match the product with the patient that has the both the highest HLA match and greatest norovirus activity through the greatest number of shared alleles. In some embodiments, the T-cell subpopulation has at least one shared allele or allele combination with norovirus activity through that allele or allele combination. In some embodiments, the T-cell subpopulation has greater than 1 shared allele or allele combination with norovirus activity through that allele or allele combination. In some embodiments, the T-cell subpopulation with the most shared alleles or allele combinations and highest specificity through those shared alleles and allele combinations is provided to a human in need thereof. For example, if T-cell subpopulation 1 is about 5/8 HLA match with the patient with norovirus activity through 3 shared alleles or allele combinations while T-cell subpopulation 2 is about 6/8 HLA match with the patient with norovirus activity through 1 shared allele the skilled practitioner would select T-cell subpopulation 1 as it has norovirus activity through a greater number of shared alleles.

### Banked T-Cell Subpopulations Directed to Norovirus Antigens

The establishment of a T-cell subpopulation bank comprising discrete, characterized T-cell subpopulations for selection and inclusion in a disclosed composition bypasses the need for an immediately available donor and eliminates the wait required for autologous T cell production. Preparing T-cell subpopulations directed to specific, norovirus antigens by using donors, for example healthy volunteers or umbilical cord blood, allows the production and banking of T-cell subpopulations readily available for administration. Because the T-cell subpopulations are characterized, the selection of suitable T-cell subpopulations can be quickly determined based on minimal information from the patient, for example HLA-subtype and, optionally norovirus expression profile.

From a single donor a T cell composition can be generated for use in multiple patients who share HLA alleles that have activity towards a specific norovirus antigen. The T-cell subpopulation bank of the present disclosure includes a population of T-cell subpopulations which have been characterized as described herein. For example, the T-cell subpopulations of the bank are characterized as to HLA-subtype and one or more of i) norovirus specificity of the T-cell subpopulation; ii) norovirus epitope(s) the T-cell subpopulation is specific to; iii) T-cell subpopulation MHC Class I and Class II restricted subsets; iv) antigenic activity through the T-cell' s corresponding HLA-allele; and v) immune effector subtype concentration, for example, the population of effector memory cells, central memory cells, γδ T-cells, CD8+, CD4+, NKT-cell.

In some embodiments, the present disclosure provides a method of generating a T-cell subpopulation bank comprising: (i) obtaining eligible donor samples; (ii) generating T-cell subpopulations specific to a single norovirus antigen; (iii) characterizing the T-cell subpopulation; (iv) cryopreserving the T-cell subpopulation; and (v) generating a database of T-cell subpopulation composition characterization data. In some embodiments, the T-cell subpopulations are stored according to their donor source. In some embodiments, the T-cell subpopulations are stored by norovirus antigen specificity. In some embodiments, the T-cell subpopulations are stored by human leukocyte antigen (HLA) subtype and restrictions.

The banked T-cell subpopulations described herein are used to comprise a disclosed composition for administration to a norovirus-infected patient following the determination of the patient's HLA subtype and, optionally, norovirus antigen expression profile of the infected norovirus strain.

Aspects and embodiments of the present disclosure will now be illustrated, by way of example, with reference to the accompanying tables and figures. Further aspects and embodiments will be apparent to those skilled in the art. All documents mentioned in this text are incorporated herein by reference.

### EXAMPLES

### Example 1. Generation of T-cell Subpopulations from Peripheral Blood Using Norovirus Antigens

### Methods

### Donors

Peripheral blood mononuclear cells (PBMCs) from healthy volunteers were obtained from Children's National Medical Center (Washington, DC) and the National Institutes of Health (Bethesda, Md.) under informed consent approved at each institution in accordance with the Declaration of Helsinki. Plasma samples were screened for the presence or absence of antibodies against norovirus by using an enzyme-linked immunosorbent assay based on several virus-like particles as previously described [24]. Stools from patients with chronic norovirus infection were obtained from patients at the NIH Clinical Center after patients signed informed consent.

### Peptides

For stimulation, custom-ordered pepmixes (peptide libraries of 15mers overlapping by 10 amino acids) were used that encompassed all entire norovirus proteins NS1-2, NS3, NS4, NS5, NS6, NS7, VP₁, and VP2 (A&A Labs, San Diego, Calif.). Protein consensus sequence was obtained from Hu/GII/Au/2012/GII.Pe_Sydney2012/NSW0514 (GenBank: JX459908).

### Generation of NSTs

PBMCs were pulsed with norovirus pepmixes pooled or individually (200 ng/peptide/15×10⁶ PBMCs) for 30 minutes at 37° C. After incubation, cells were resuspended with IL-4 (400 IU/ml; R&D Systems, Minneapolis, Minn.) and IL-7 (10 ng/ml; R&D Systems) in CTL media consisting of 45% RPMI (GE Healthcare, Logan, Utah), 45% Click's medium (Irvine Scientific, Santa Ana, Calif.), 10% human AB serum (Gemini BioProducts, West Sacramento, Calif.) and supplemented with 2 mmol/1 GlutaMax (Gibco, Grand Island, N.Y.), and transferred to a G-Rex 10 device (Wilson Wolf, Minneapolis, Minn.). Media and cytokines were replenished on day 7. On day 10, cells were harvested and evaluated for antigen specificity and functionality.

### IFN-γ Enzyme-Linked Immunospot (ELISpot) Assay

Antigen specificity of T-cells was measured with IFN-γ ELISpot (Millipore, Burlington, Mass.). T-cells were plated at 1×10⁵/well with no peptide, actin (control), or each of the individual norovirus pepmixes (200 ng/peptide/well). IFN-γ spots forming cells (SFC) were quantified by Zellnet Consulting (Fort Lee, NJ).

### Multiplex Cytokine Assay

NSTs were plated on 1×10⁵/well in 96-well plate, stimulated with pooled pepmixes (200 ng/peptide/well) or control actin peptide, and incubated overnight. Supernatants were harvested and the cytokine profile analysis was performed using the Bio-plex Pro Human 17-plex Cytokine Assay kit (Bio-Rad, Hercules, Calif.).

### Immunophenotyping

NSTs were stained with fluorophore-conjugated antibodies against CD3, CD4, CD8, CD14, CD16, CD19, CD25, CD45RO, CD62L, CD127, CCR7, PD-1, LAG-3, TIM-3, and CTLA-4 (Miltenyi Biotec, Bergisch Gladbach, Germany; BioLegend, San Diego, Calif.). All samples were acquired on a CytoFLEX cytometer (Beckman Coulter, Brea, Calif.) and the data was analyzed with FlowJo X (FlowJo LLC, Ashland, Oreg.).

### Intracellular Cytokine Staining

A total of 1×10⁶ NSTs were plated in a 96-well plate and stimulated with pooled pepmixes or individual peptide (200 ng/peptide/well) or actin (control) in the presence of Brefeldin A (Golgiplug; BD Biosciences, San Jose, Calif.) and CD28/CD49d (BD Biosciences) for 6 hours. T-cells were fixed, permeabilized with Cytofix/Cytoperm solution (BD Biosciences) and stained with IFN-γ and TNF-α antibodies (Miltenyi Biotec).

### Epitope Mapping

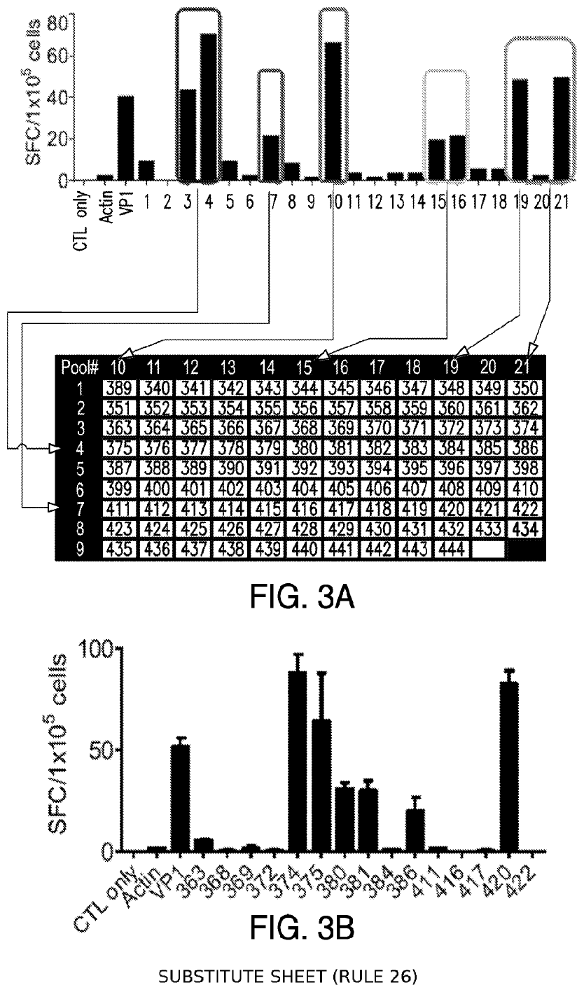

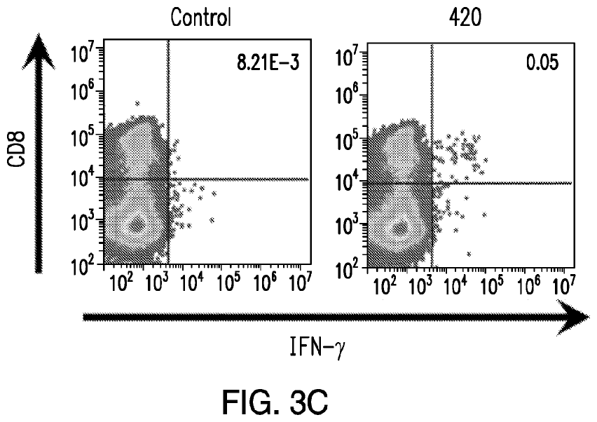

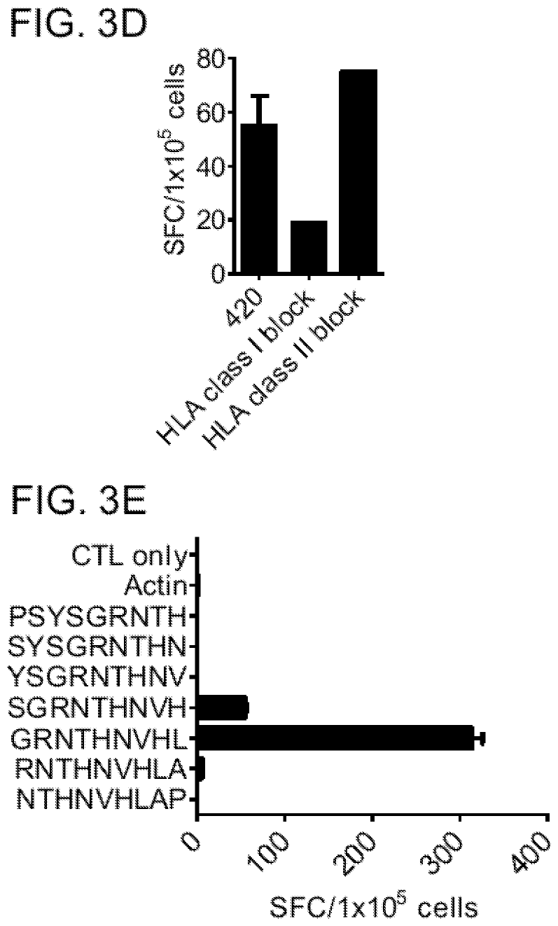

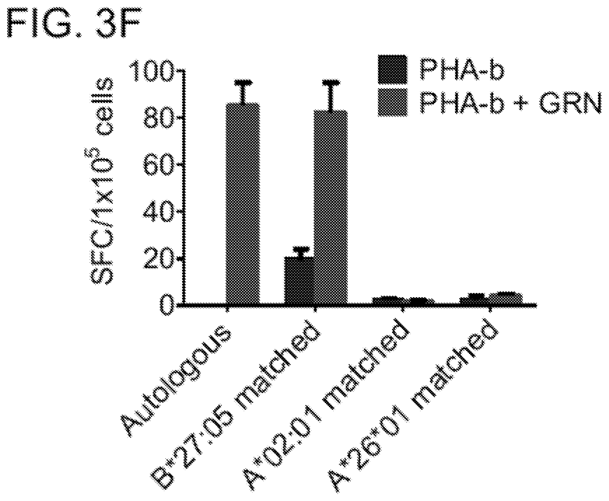

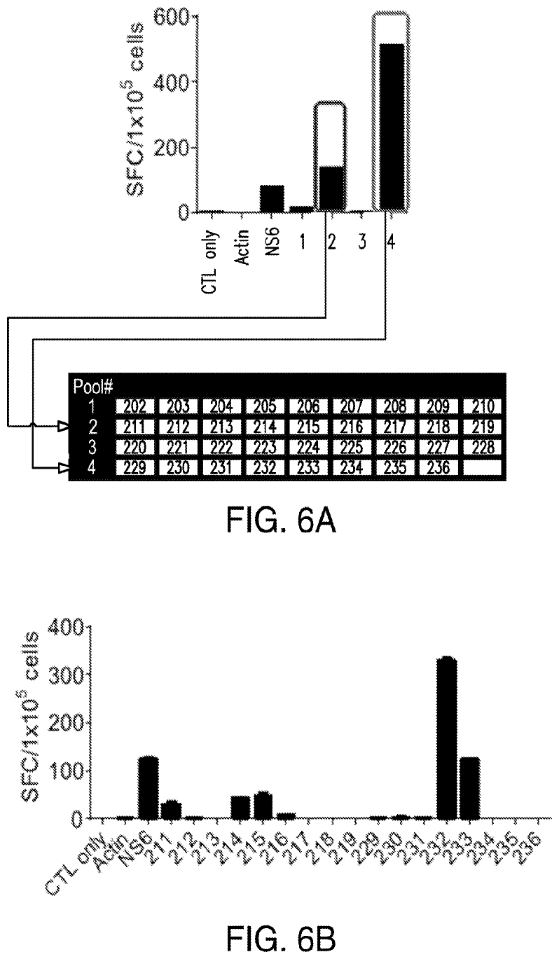

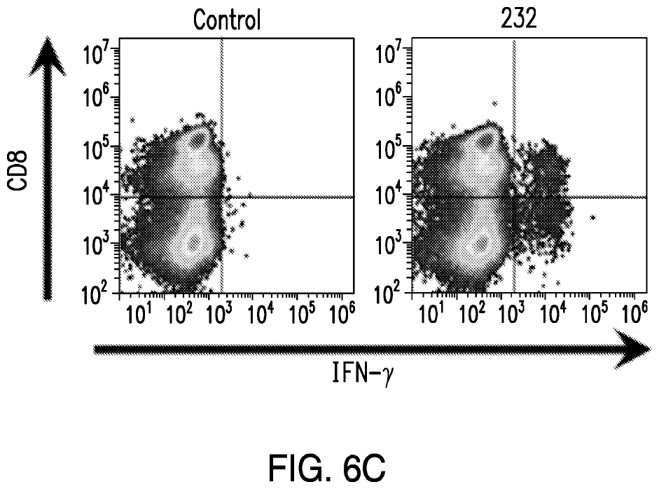

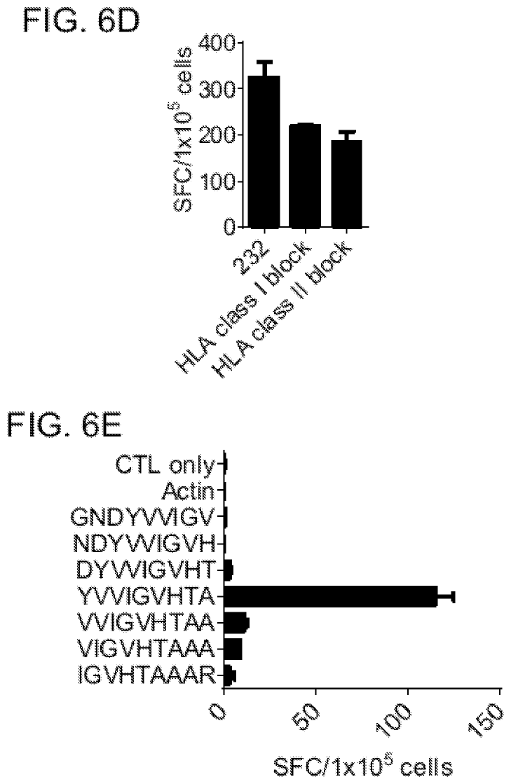

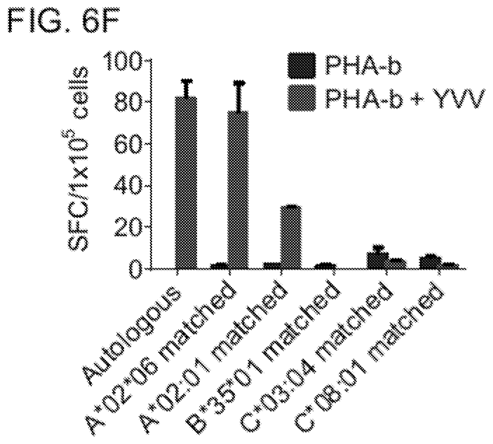

NSTs were tested for specificity to NS6 and VP1 individual peptides in IFN-γ ELISpot. The NS6 and VP1 15mer peptides were pooled according to the matrix as shown in FIG. 6A and FIG. 3A, respectively. Cross-reactive pools were analyzed and individual peptides were tested to confirm epitope specificity. To identify minimal epitopes, a series of 9mer peptides overlapping by 8 amino acids spanning immunogenic 15mer peptides were generated. The HLA-restriction of antigen recognition was tested using blocking antibodies against HLA class I and II (Dako Agilent, Santa Clara, Calif.). To determine the restricted HLA allele, NSTs were plated at 1×10⁵/well with partially HLA-matched phytohemagglutinin (PHA)-treated lymphoblasts (PHA-blasts, 25 Gy irradiated) either alone or pulsed with peptide (10 μg/ml).

### Data Analysis

Results were evaluated using descriptive statistics (means, standard error of the mean [SEM], and ranges). Two-tailed Student's t test was used to compare the 2 groups. Data analysis was performed in Graphpad Prism (GraphPad Software, La Jolla, Calif.).

### Results

NSTs can be Expanded from Healthy Donors

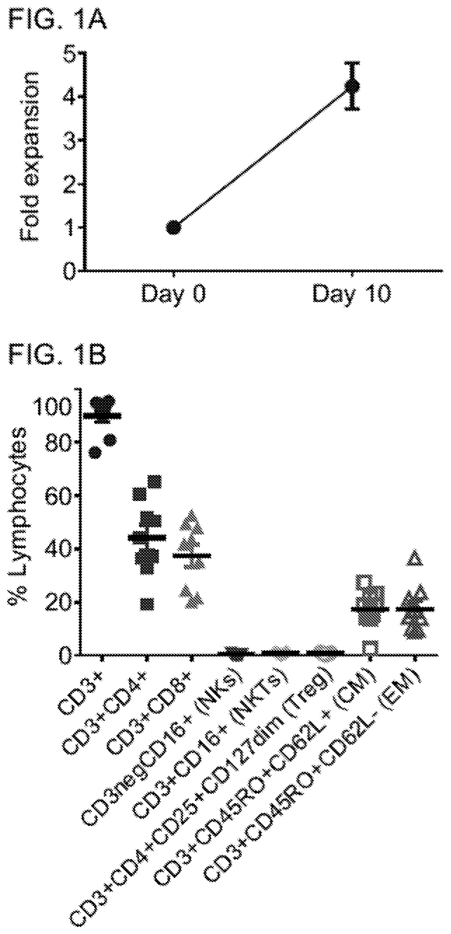

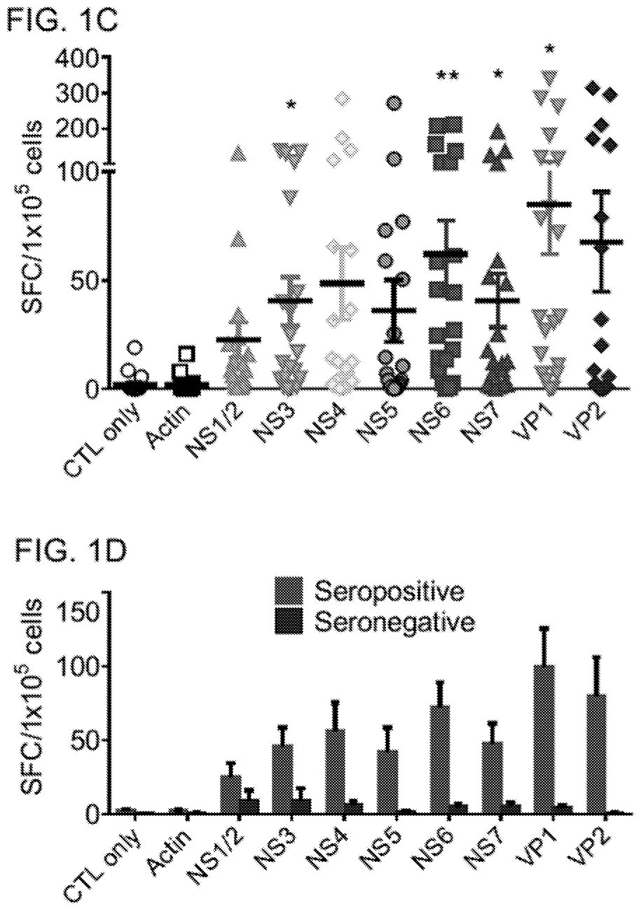

To evaluate whether NSTs can be expanded from healthy donors, PBMCs were stimulated with norovirus pepmixes spanning all antigens (NS1-2, NS3, NS4, NS5, NS6, NS7, VP1, and VP2) and cultured for 10 days in the presence of IL-4 and IL-7. After stimulation, a mean 4.2±0.5 fold increase (range, 1.0-8.8) was achieved in total cell numbers (FIG. 1A). Phenotyping analysis revealed that these expanded cells comprised CD3⁺ T-cells (mean±SEM, 90.0±2.3%) with a mixture of CD4⁺ T-cells (44.2±4.8%) and CD8⁺ T-cells (37.3±4.1%) (FIG. 1B). There was no outgrowth of NK cells and regulatory T-cells. The expanded T-cells expressed both central memory (17.2±2.3%) and effector memory (17.4±2.9%) markers. The specificity of the expanded T-cells was next determined against norovirus antigens by IFN-γ ELISpot assay. T-cells specifically released IFN-γ against NS1/2 (22.7±8.0 SFC/1×10⁵ cells; P=0.10), NS3 (40.4±11.1; P=0.01), NS4 (48.6±16.7; P=0.06), NS5 (35.9±14.4; P=0.14), NS6 (62.1±15.3; P<0.01), NS7 (40.7±12.3; P=0.03), VP1 (85.2±23.0; P=0.01), and VP2 (67.8±23.0; P=0.06), compared to actin control (1.6±0.8) (FIG. 1C). An association between serostatus against norovirus and the quality of T-cell responses was examined. Importantly, NSTs were only detected in T-cell products derived from seropositive donors while the T-cells from seronegative donors did not demonstrate specificity (FIG. 1D). Together, these data suggest that T-cells targeting multiple norovirus antigens can be readily ex vivo expanded from seropositive donors to clinically relevant numbers.

### Characterization of NSTs

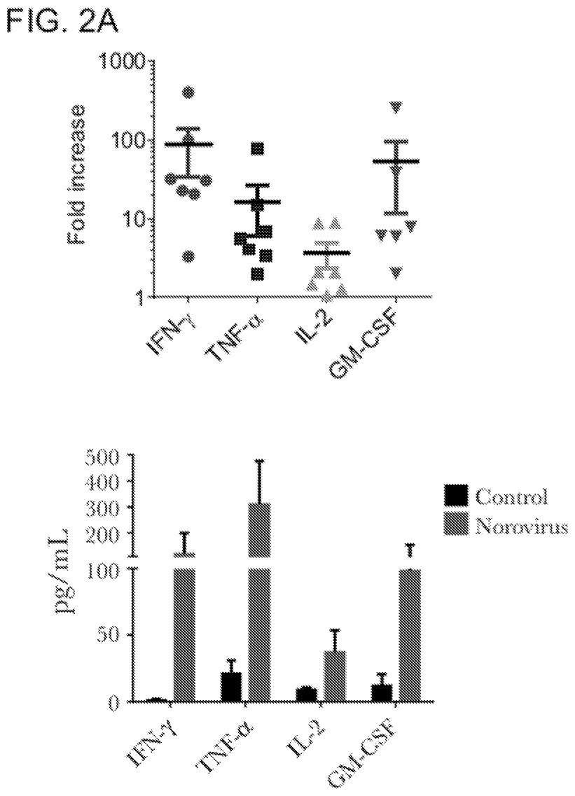

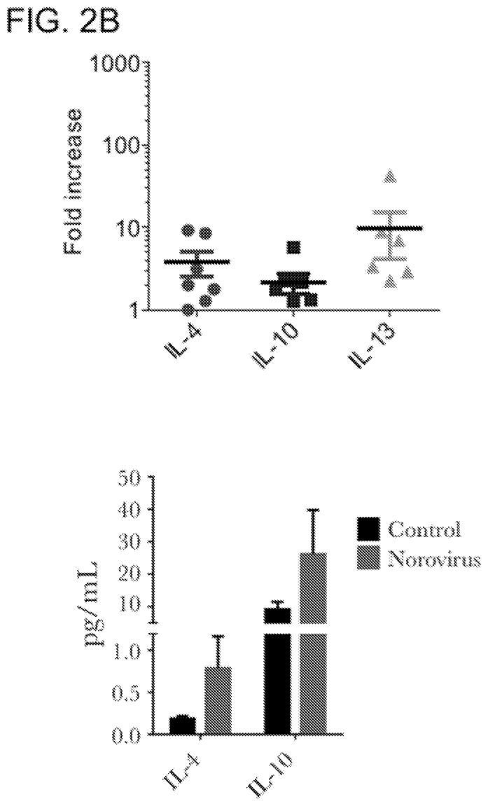

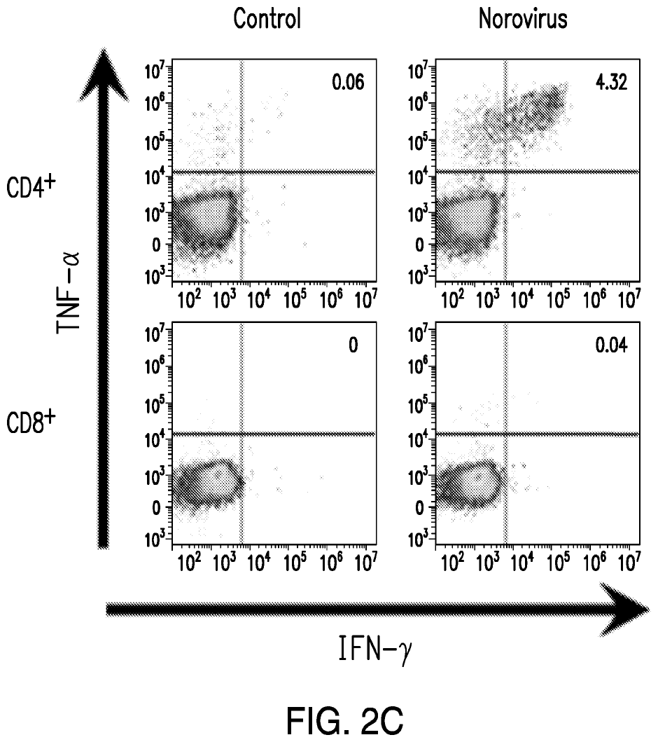

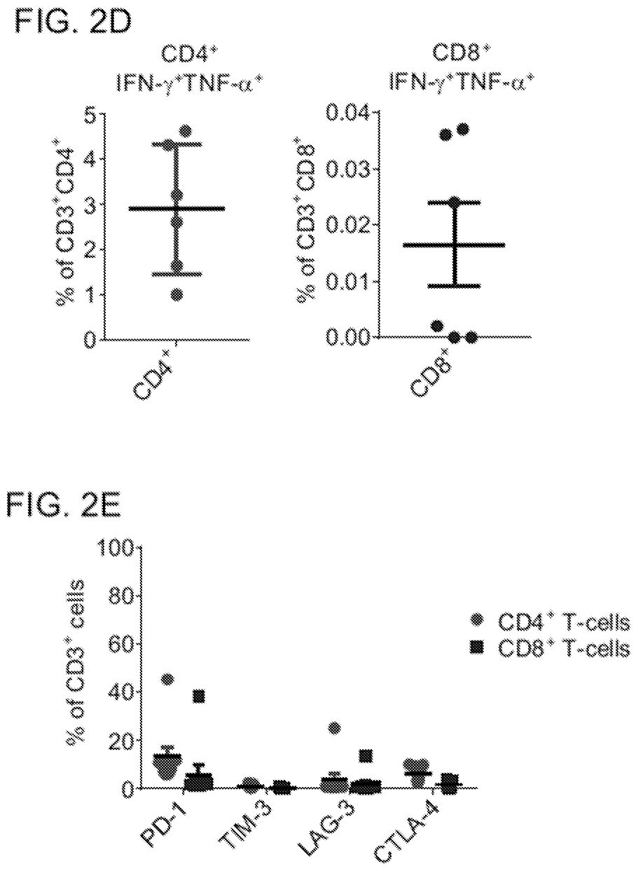

Recent studies suggest that polyfunctional antigen-specific T-cells have improved cytolytic function and superior in vivo activity [33]. The production of multiple pro-inflammatory cytokines to determine the polyfunctionality of NSTs was evaluated. NSTs released IFN-γ (87.4±53.8-fold), TNF-α (16.3±10.4-fold), IL-2 (3.6±1.3-fold) and GM-CSF (52.6±40.9-fold) upon stimulation with norovirus pepmixes compared to actin control (FIG. 2A) with minimal production of regulatory cytokines including IL-4, IL-10, and IL-13 (FIG. 2B). To further evaluate whether NSTs produce more than one cytokine, intracellular IFN-γ and TNF-α staining was performed, gating on CD4⁺ and CD8⁺ T-cells (FIG. 2C). After restimulation with norovirus pepmixes, T-cells were predominantly detected in the CD4⁺ T-cell compartment (2.9±0.5%) with a limited CD8⁺ component (FIG. 2D). Co-inhibitory receptors including PD-1, TIM-3, LAG-3, and CTLA-4 on CD4⁺ and CD8⁺ T-cells were measured. Despite restimulation with norovirus antigens, flow analysis revealed low expression of co-inhibitory molecules (FIG. 2E). Together, these data indicate that the ex vivo expanded NSTs are polyclonal and polyfunctional.

### Identification of T-Cell Epitopes

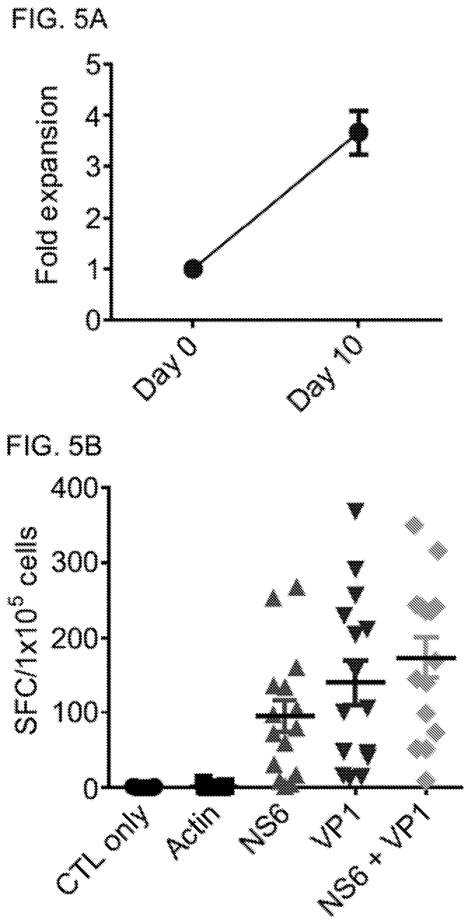

The hierarchy of immunodominance based on the number of responding donors and the magnitude of reactive cells is shown in Table 1. The two most immunogenic antigens, NS6 and VP1, were selected for further epitope mapping. To avoid antigen competition, PBMCs from healthy donors were stimulated with NS6 and VP1 pepmixes and cultured for 10 days with IL-4 and IL-7. The resultant T-cells showed a mean expansion of 3.4±0.4-fold (range, 1.9-6.7) in total cell numbers (FIG. 5A). Of note, they were specific for both NS6 (95.1±21.6 SFC/1×10⁵ cells) and VP1 antigens (140.0±29.4) at higher frequencies of specific cells compared to those achieved using whole antigens (FIG. 5B). Next, the breadth of epitope specificity of NS6 and VP1 were analyzed utilizing these NS6/VP1-specific T-cells. A representative example of mapping for VP1 is shown in FIG. 3A-3F. In this approach, 106 15mers spanning VP1 protein was divided into 21 mini-pools such that each peptide is uniquely present in 2 pools. Using this method, the T-cell product recognized 8 mini-pools (3, 4, 7, 10, 15, 16, 19 and 21), and individual 15mers that were present in the 2 of the peptides were selected (FIG. 3A). Testing of the single peptides by IFN-γ ELISpot revealed recognition of the single peptides 374, 375, 380, 381, 386, and 420 (FIG. 3B). HLA-restriction of these peptides was evaluated with intracellular IFN-γ staining. CD8⁺ T-cells released INF-γ in response to 15mer peptide 420 PSYSGRNTHNVHLAP (PSY), indicating that this peptide was HLA class I-restricted epitope (FIG. 3C). This was further confirmed by using HLA blocking experiment in IFN-γ ELISpot (FIG. 3D). The minimal 9mer epitope and restricted HLA class I allele of this peptide was determined. The NST was rescreened against a panel of 9mer peptides overlapping by 8 amino acids spanning the 15mer PSY peptide. The T-cells secreted IFN-γ upon stimulation with GRNTHNVHL (GRN), confirming GRN was the minimal 9mer epitope (FIG. 3E). Finally, to determine the restricted HLA allele of GRN, the predictive algorithm (NetMHC, cbs.dtu.dk/services/NetMHCpan/) was used to predict that GRN would bind strongly to HLA-B*27:05 among the HLA type of the donor. This predicted HLA restriction was tested using autologous and partially HLA-matched peptide-pulsed PHA blasts in an ELISpot assay. NSTs recognized peptide-pulsed PHA blasts matched for HLA-B*27:05 with no reactivity against PHA-blasts matched at other HLA alleles (FIG. 3F). The breadth of specificity to NS6 was tested using the same approach (FIG. 6A-6F). The complete NS6/VP1 mapping data of CD4⁺ and CD8-restricted epitopes in 11 donors tested is summarized in Tables 2 and 3, respectively. While specificity was detected predominantly in CD4⁺ T-cells, three novel CD8-restricted epitopes were identified.

TABLE 1
Hierarchy of immunodominance among norovirus antigens

| | | Response, SFC/1 × 10⁵ cells | | |
|---|---|---|---|---|
| Antigen | Responders (n = 20)^{a} | Mean ± SEM | Median (Range) | |
| NS1/2 | 5 | 23 ± 8 | 9 | (0-130) |
| NS3 | 5 | 40 ± 11 | 15 | (0-138) |
| NS4 | 7 | 49 ± 17 | 13 | (0-284) |
| NS5 | 6 | 36 ± 14 | 6 | (0-271) |
| NS6 | 11 | 62 ± 15 | 36 | (0-213) |
| NS7 | 7 | 41 ± 12 | 12 | (0-191) |
| VP1 | 13 | 85 ± 23 | 33 | (0-340) |
| VP2 | 8 | 68 ± 23 | 7 | (0-313) |

Abbreviations: SFC, spot forming cells; SEM, standard error of the mean.
^{a}Responses that exceeded 5 × actin control and were at least 20 SFC/1 ×10⁵ cells were regarded as significant.

TABLE 2
Peptide sequences of novel CD4⁺ T-cell epitopes identified in NS6 and VP1

| Norovirus | | Amino acid | | | |
|---|---|---|---|---|---|
| antigen | Peptide sequence | location | HLA-DRB1 | HLA-DQB1 | HLA-DPB1 |
| NS6 | GSGWGFWVSPSLFIT | 1021-1035 | 11:01, 07:01 | 02:02, 03:01 | 02:01, 04:01 |
| | | | 11:04, 07:01 | 02:02, 03:01 | — |
| | | | 04:05, 07:01 | 02:02, 04:01 | 02:01, 05:01 |
| | FWVSPSLFITSTHVI | 1026-1040 | 11:01, 07:01 | 02:02, 03:01 | 02:01, 04:01 |
| | | | 11:04, 07:01 | 02:02, 03:01 | — |
| | | | 04:05, 07:01 | 02:02, 04:01 | 02:01, 05:01 |
| | SLFITSTHVIPQSAK | 1031-1046 | 11:04, 07:01 | 02:02, 03:01 | — |
| | PQSAKEFFGVPIKQI | 1041-1955 | 04:05, 07:01 | 02:02, 04:01 | 02:01, 05:01 |
| | EFFGVPIKQIQIHKS | 1046-1060 | 04:05, 07:01 | 02:02, 04:01 | 02:01, 05:01 |
| | PIKQIQIHKSGEFCR | 1051-1065 | 15:01, 13:01 | 06:02, 06:03 | 02:01, 04:01 |
| | | | 15:01, 12:02 | 03:01, 06:02 | 05:01, 05:01 |
| | | | 01:01, 13:01 | 05:01, 06:03 | 03:01, 03:01 |
| | | | 11:01, 13:02 | 03:01, 06:04 | 04:01, 14:01 |
| | LRFPKPIRTDVTGMI | 1066-1080 | 15:01, 12:02 | 03:01, 06:02 | 05:01, 05:01 |
| | | | 11:01, 13:02 | 03:01, 06:04 | 04:01, 14:01 |
| | PIRTDVTGMILEEGA | 1071-1085 | 15:01, 12:02 | 03:01, 06:02 | 05:01, 05:01 |
| | | | 11:01, 13:02 | 03:01, 06:04 | 04:01, 14:01 |
| | LEEGAPEGTVATLLI | 1081-1095 | 11:01, 07:01 | 02:02, 03:01 | 02:01, 04:01 |
| | PEGTVATLLIKRPTG | 1086-1100 | 11:01, 07:01 | 02:02, 03:01 | 02:01, 04:01 |
| | GNDYVVIGVHTAAAR | 1156-1170 | 11:01, 07:01 | 02:02, 03:01 | 02:01, 04:01 |
| | | | 03:01, 04:04 | 02:01, 03:02 | 02:01, 03:01 |
| | | | 15:01, 12:02 | 03:01, 06:02 | 05:01, 05:01 |
| | | | 04:04, 11:01 | 03:01, 03:02 | 04:01, 04:01 |
| | | | 11:01, 13:02 | 03:01, 06:04 | 04:01, 14:01 |
| | VIGVHTAAARGGNTV | 1161-1175 | 15:01, 12:02 | 03:01, 06:02 | 05:01, 05:01 |
| | | | 04:04, 11:01 | 03:01, 03:02 | 04:01, 04:01 |
| | | | 11:01, 13:02 | 03:01, 06:04 | 04:01, 14:01 |
| VP1 | PVVGAAIAAPVAGQQ | 31-45 | 11:01, 07:01 | 02:02, 03:01 | 02:01, 04:01 |
| | LGPDLNPYLSHLARM | 81-95 | 15:01, 13:01 | 06:02, 06:03 | 02:01, 04:01 |
| | VRNNFYHYNQSNDPT | 161-175 | 11:01, 07:01 | 02:02, 03:01 | 02:01, 04:01 |
| | IKLIAMLYTPLRANN | 176-190 | 15:01, 13:01 | 06:02, 06:03 | 02:01, 04:01 |
| | | | 01:01, 13:01 | 05:01, 06:03 | 03:01, 03:01 |
| | MLYTPLRANNAGDDV | 181-195 | 01:01, 13:01 | 05:01, 06:03 | 03:01, 03:01 |
| | AGDDVFTVSCRVLTR | 186-200 | 11:01, 13:02 | 03:01, 06:04 | 04:01, 14:01 |
| | PSPDFDFIFLVPPTV | 206-220 | 01:01, 11:01 | 03:01, 05:01 | 04:01, 04:02 |
| | | | 10:01, 04:11 | 03:02, 05:01 | 04:02, 10:01 |
| | | | 01:01, 13:01 | 05:01, 06:03 | 03:01, 03:01 |
| | DFIFLVPPTVESRTK | 211-225 | 01:01, 11:01 | 03:01, 05:01 | 04:01, 04:02 |
| | | | 10:01, 04:11 | 03:02, 05:01 | 04:02, 10:01 |
| | | | 01:01, 13:01 | 05:01, 06:03 | 03:01, 03:01 |
| | EMTNSRFPIPLEKLF | 236-250 | 03:01, 04:04 | 02:01, 03:02 | 02:01, 03:01 |
| | | | 11:01, 13:02 | 03:01, 06:04 | 04:01, 14:01 |
| | RFPIPLEKLFTGPSS | 241-255 | 03:01, 04:04 | 02:01, 03:02 | 02:01, 03:01 |
| | | | 11:01, 13:02 | 03:01, 06:04 | 04:01, 14:01 |
| | SRNYTMNLASQNWND | 296-310 | 10:01, 04:11 | 03:02, 05:01 | 04:02, 10:01 |
| | | | 11:04, 07:01 | 02:02, 03:01 | — |
| | | | 04:04, 11:01 | 03:01, 03:02 | 04:01, 04:01 |
| | | | 11:01, 13:02 | 03:01, 06:04 | 04:01, 14:01 |
| | EWVQYFYQEAAPAQS | 456-470 | 01:01, 11:01 | 03:01, 05:01 | 04:01, 04:02 |
| | APAQSDVALLRFVNP | 466-480 | 11:01, 07:01 | 02:02, 03:01 | 02:01, 04:01 |
| | DVALLRFVNPDTGRV | 471-485 | 11:01, 07:01 | 02:02, 03:01 | 02:01, 04:01 |
| | | | 04:05, 07:01 | 02:02, 04:01 | 02:01, 05:01 |
| | DTGRVLFECKLHKSG | 481-495 | 03:01, 04:04 | 02:01, 03:02 | 02:01, 03:01 |
| | VNQFYTLAPMGNGTG | 526-540 | 10:01, 04:11 | 03:02, 05:01 | 04:02, 10:01 |

TABLE 3
Peptide sequences of novel CD8⁺ T-cell epitopes identified in NS6
and VP1

| Norovirus | | Amino acid | |
|---|---|---|---|
| antigen | Peptide sequence | location | HLA restriction |
| NS6 | YVVIGVHTA | 1159-1167 | A*02:06 |
| VP1 | GRNTHNVHL | 410-418 | B*27:05 |
| VP1 | FPGEQLLFF | 426-434 | B*35:01 |

### Cross-Reactivity of NSTs

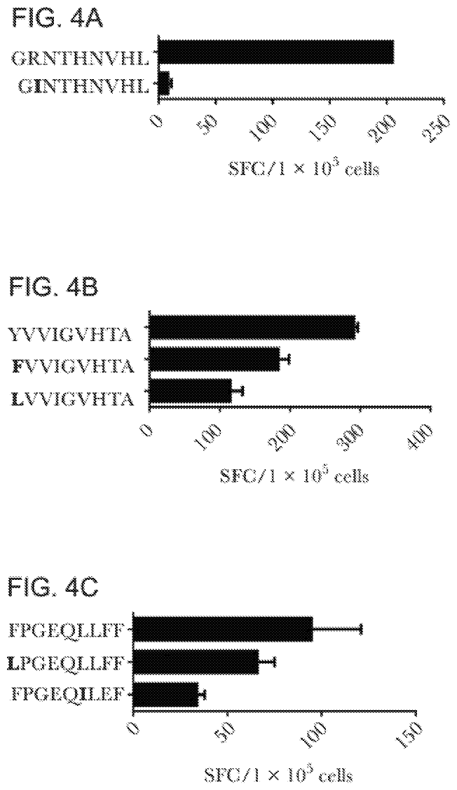

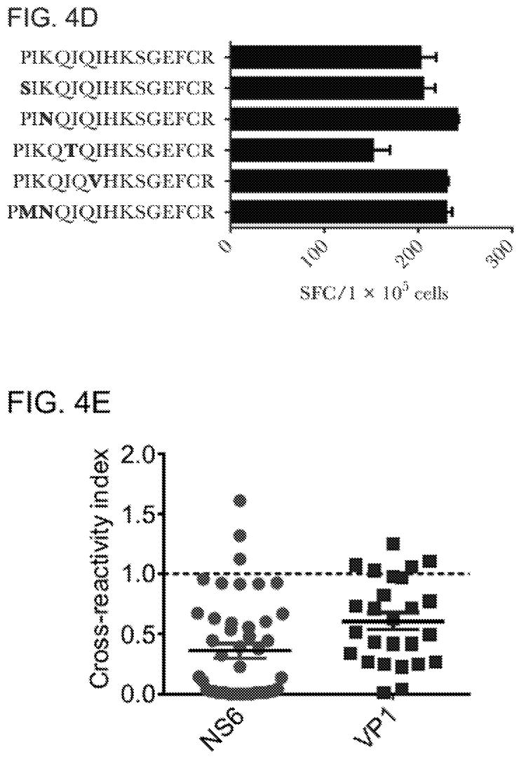

The norovirus peptide library used in this study was based on the GII.4 Sydney/2012 strain, which is currently the most prevalent strain worldwide. However, many other norovirus genotypes, recombinants, and strain variants cocirculate, and the RNA genome can evolve rapidly under selective pressure. To assess whether the newly identified immunogenic epitopes were conserved between different norovirus strains, the amino acid sequences of NS6/VP1 epitopes present in the NCBI database were compared to those of clinical isolates from patients with chronic norovirus infection. Among identified epitopes including both HLA class I and II-restricted, 1 to 7 amino acid sequences were altered in 22 of the 31 epitopes (Table 4). The cross-reactivity of NSTs against variant epitopes in IFN-γ ELISpot was evaluated. While the recognition of CD8⁺-restricted epitopes GRN was disrupted when amino acid was altered at an anchor site (FIG. 4A), NSTs widely responded to variants of CD8⁺ (FIG. 4B) and CD4⁺-restricted epitopes (FIG. 4C). Cross-reactivity index (variant response/GII.4 Sydney/2012 response) was used to normalize across different mutated epitopes, with 1 indicating an equal response to original and variant peptide (Table 4). Importantly, responses were highly cross-reactive against different norovirus strains and mutated epitopes in both NS6 (cross-reactivity index, 0.36±0.06) and VP1 (0.60±0.06) (FIG. 4D).

TABLE 4
Mutated peptides eliciting T-cell responses

| | | | | | | | Cross- |
|---|---|---|---|---|---|---|---|
| | | HLA | Amino acid | | | | reactivity |
| Antigen | Peptide | restriction | location | Variant | Strain/isolate | GenBank | index |
| NS6 | YVVIGVHTA | Class I | 1159-1167 | FVVIGVHTA | Norovirus Hu/GII.3/ | AEK99648.1 | 0.628 |
| | | | | | Maastricht021/2006/NL | | (n = 1) |
| | | | | LVVIGVHTA | Norovirus GII.17 | YP_009538338.1 | 0.393 |
| | | | | | | | (n = 1) |
| NS6 | GSGWGFWVSPS | Class II | 1021-1035 | GTGWGFWVSPS | Norovirus Hu/GII.3/ | AEK99648.1 | 1.318 |
| | LFIT | | | LFIT | Maastricht021/2006/NL | | (n = 1) |
| NS6 | PQSAKEFFGVP | Class II | 1041-1955 | PQGAKEFFGVP | Norovirus Hu/GII.4-2006b/ | AEK99652.1 | 0.590 |
| | IKQI | | | IKQI | Nijmegen115/2006/NL | | (n = 1) |
| | | | | PQGAQEFFGVP | Norovirus Hu/GII.b/ | AEK99654.1 | 0.326 |
| | | | | IKQI | Nieuwegein004/2006/NL | | (n = 1) |
| | | | | PQGAKEFFGVS | Norovirus GII | APE73914.1 | 0.108 |
| | | | | IKQI | | | (n = 1) |
| | | | | PQGAKEFFGVP | Norwalk virus | CAE47528.2 | 0.145 |
| | | | | IKQT | | | (n = 1) |
| | | | | PQGAQEFFGVS | Norwalk-like virus | YP_009238490.1 | 0.024 |
| | | | | IKQI | | | (n = 1) |
| | | | | PQGTQEFFGVS | Norovirus Hu/GII/21-5/ | BAV24787.1 | 0.024 |
| | | | | IKQI | Tokyo/1975/JPN | | (n = 1) |
| | | | | PQGSQEFFGVS | Norovirus Hu/GII.3/ | AGH7608.1 | 0.036 |
| | | | | IKQI | HK46/1977/CHN | | (n = 1) |
| | | | | PAGITEAFGVP | Norovirus GII | YP 009518848.1 | 0.012 |
| | | | | IKQI | | | (n = 1) |
| | | | | PKGITEAFGVP | Norovirus GII.17 | YP 009538338.1 | 0 |
| | | | | INQI | | | (n = 1) |
| | | | | PKGITEAFGVP | Norovirus Hu/GII.3/ | AEK99648.1 | 0 |
| | | | | MNQI | Maastricht021/2006/NL | | (n = 1) |
| NS6 | EFFGVPIKQIQ | Class II | 1046-1060 | EAFGVPIKQIQ | Norovirus GII | YP_009518848.1 | 0.444 |
| | IHKS | | | IHKS | | | (n = 1) |
| | | | | EFFGVSIKQIQ | Norovirus Hu/GII/21-5/ | BAV24787.1 | 0.138 |
| | | | | IHKS | Tokyo/1975/JPN | | (n = 1) |
| | | | | EFFGVPIKQTQ | Norwalk virus | CAE47528.2 | 0.444 |
| | | | | IHKS | | | (n = 1) |
| | | | | EFFGVPIKQIQ | Norovirus Hu/GII.4-2006b/ | AEK99652.1 | 1.611 |
| | | | | VHKS | Nijmegen115/2006/NL | | (n = 1) |
| | | | | EAFGVPINQIQ | Norovirus GII.17 | YP_009538338.1 | 0 |
| | | | | IHKS | Norovirus Hu/GII.3/ | | (n = 1) |
| | | | | EAFGVPMNQIQ | Maastricht021/2006/NL | AEK99648.1 | 0 |
| | | | | IHKS | | | (n = 1) |
| NS6 | PIKQIQIHKSG | Class II | 1051-1065 | SIKQIQIHKSG | Norovirus Hu/GII/21-5/ | BAV24787.1 | 0.914 |
| | EFCR | | | EFCR | Tokyo/1975/JPN | | (n = 3) |
| | | | | PINQIQIHKSG | Norovirus GII.17 | YP_009538338.1 | 0.666 |
| | | | | EFCR | Norwalk virus | | (n = 3) |
| | | | | PIKQTQIHKSG | | CAE47528.2 | 0.601 |
| | | | | EFCR | | | (n = 3) |
| | | | | PIKQIQVHKSG | Norovirus Hu/GII.4/ | AEK99653.1 | 0.955 |
| | | | | EFCR | Kreischa23773/2007/DE | | (n = 3) |
| | | | | PMNQIQIHKSG | Norovirus Hu/GII.3/ | AEK99648.1 | 0.671 |
| | | | | EFCR | Maastricht021/2006/NL | | (n = 3) |
| NS6 | LRFPKPIRTDV | Class II | 1066-1080 | LRFPKPIRTDV | Norovirus Hu/GII/53-1/ | BAV24793.1 | 0.921 |
| | TGMI | | | AGMI | Tokyo/1980/JPN | | ( n= 2) |
| | | | | FRFPKPIRPDV | Norovirus GII | YP_009518848.1 | 0.377 |
| | | | | TGMI | | | (n = 2) |
| | | | | LRFPKPIRPDV | Norovirus Hu/GII.3/ | AEK99648.1 | 0.476 |
| | | | | SGMI | Maastricht021/2006/NL | | (n = 2) |
| NS6 | PIRTDVTGMIL | Class II | 1071-1085 | PIRPDVTGMIL | Norovirus GII | YP_009518848.1 | 0.533 |
| | EEGA | | | EEGA | | | (n = 2) |
| | | | | PIRTDVAGMIL | Norovirus Hu/GII/53-1/ | BAV24793.1 | 0.555 |
| | | | | EEGA | Tokyo/1980/JPN | | (n = 2) |
| | | | | PIRPDVSGMIL | Norovirus Hu/GII.3/ | AEK99648.1 | 0.228 |
| | | | | EEGA | Maastricht021/2006/NL | | (n = 2) |
| NS6 | LEEGAPEGTVA | Class II | 1081- | LEEGAPGGTVA | Norovirus Hu/GII.4-2002/ | AEK99649.1 | 0 |
| | TLLI | | 1095 | TLLI | WeertE022/2002/NL | | (n = 1) |
| | | | | LEEGAPEGTVV | Norwalk-like virus | YP_009238490.1 | 0.044 |
| | | | | TLLI | | | (n = 1) |
| | | | | LEEGAPEGTVA | Norovirus GII | YP_009518848.1 | 0.014 |
| | | | | TVLI | | | (n = 1) |
| | | | | LEEGAPEGTVA | Norovirus Hu/GII.3/ | AEK99648.1 | 0.014 |
| | | | | SILI | Maastricht021/2006/NL | | (n = 1) |
| | | | | LEEGAPEGTVV | Norovirus GII.17 | YP_009538338.1 | 0 |
| | | | | SILI | | | (n = 1) |
| NS6 | PEGTVATLLIK | Class II | 1086- | PEGTVVTLLIK | Norwalk-like virus | YP_009238490.1 | 0.013 |
| | RPTG | | 1100 | RPTG | | | (n = 1) |
| | | | | PEGTVATVLIK | Norovirus GII | YP_009518848.1 | 0.013 |
| | | | | RPTG | | | (n = 1) |

| | | | | PEGTVATLLIK | Norovirus Hu/GII.4-2006b/ | AEK99652.1 | 0.026 |
|---|---|---|---|---|---|---|---|
| | | | | RSTG | Nijmegen115/2006/NL | | (n = 1) |
| | | | | PGGTVATLLIK | Norovirus Hu/GII.4-2002/ | AEK99649.1 | 0.013 |
| | | | | RSTG | WeertE022/2002/NL | | (n = 1) |
| | | | | PEGTVVTLLIK | Norovirus Hu/GII.4/ | AEK99653.1 | 0.013 |
| | | | | RSTG | Kreischa23773/2007/DE | | (n = 1) |
| | | | | PEGTVASILIK | Norovirus Hu/GII.3/ | AEK99648.1 | 0 |
| | | | | RTTG | Maastricht021/2006/NL | | (n = 1) |
| | | | | PEGTVVSILIK | Norovirus GII.17 | YP_009538338.1 | 0.026 |
| | | | | RTTG | | | (n = 1) |
| NS6 | GNDYVVIGVHT | Class II | 1156-1170 | ENDYVVIGVHT | Norovirus Hu/GII/52-2/ | BAV24790.1 | 1.124 |
| | AAAR | | | AAAR | Tokyo/1980/JPN | | (n = 5) |
| | | | | GNDFVVIGVHT | Norovirus Hu/GII.3/ | AEK99648.1 | 0.925 |
| | | | | AAAR | Maastricht021/2006/NL | | (n = 5) |
| | | | | GNDLVVIGVHT | Norovirus GII.17 | YP_009538338.1 | 0.918 |
| | | | | AAAR | | | (n = 5) |
| VP1 | GRNTHNVHL | Class I | 410-418 | GINTHNVHL | NIHIC_76.1 | | 0.039 |
| | | | | | | | (n = 1) |
| VP1 | FPGEQLLFF | Class I | 426-434 | LPGEQLLFF | NIHIC_76.1 | | 0.698 |
| | | | | | | | (n = 1) |
| | | | | FPGEQILFF | NIHIC_78.3 | | 0.359 |
| | | | | | | | (n = 1) |
| VP1 | PVVGAAIAAPV | Class II | 31-45 | PVAGAAIAAPV | Norovirus Hu/GII.4/ | AGS08109.1 | 1.250 |
| | AGQQ | | | AGQQ | NSW628G/2012/AU | | (n = 1) |
| VP1 | LGPDLNPYLSH | Class II | 81-95 | LGPGLNPYLSH | NIHIC_76.1 | | 0.823 |
| | LARM | | | LARM | | | (n = 1) |
| | | | | LGPDLNPYLSH | NIHIC_85.1 | | 0.623 |
| | | | | LSRM | | | (n = 1) |
| VP1 | VRNNFYHYNQS | Class II | 161-175 | VRNNFYHYNQS | Norovirus GII.4 | ATI15266.1 | 0.977 |
| | NDPT | | | NDST | | | (n = 1) |
| | | | | VRNNFYHYNQI | NIHIC_85.1 | | 0.227 |
| | | | | NEPT | | | (n = 1) |
| VP1 | IKLIAMLYTPL | Class II | 175-190 | LKLIAMLYTPL | NIHIC_70.1 | | 0.494 |
| | RANN | | | RANN | | | (n = 1) |
| VP1 | DFIFLVPPTVE | Class II | 211-225 | DFIFLVPPTIE | NIHIC_85.1 | | 1.061 |
| | SRTK | | | SRTK | | | (n = 1) |
| | | | | DFIFLVPPTVE | NIHIC_70.2 | | 0.417 |
| | | | | SKTK | | | (n = 1) |
| VP1 | RFPIPLEKLFT | Class II | 241-255 | RFPIPLEKLFT | Norovirus Hu/GII.4/ | AGS08167.1 | 1.103 |
| | GPSS | | | GPSN | NLV-12-65/2012/NZ | | (n = 2) |
| VP1 | SRNYTMNLASQ | Class II | 295-310 | SHNYTMNLASQ | NIHIC_78.4 | | 1.027 |
| | NWND | | | NWND | | | (n = 3) |
| | | | | SRNYSMNLASQ | NIHIC_66.5 | | 0.709 |
| | | | | NWND | | | (n = 1) |
| | | | | SRNYSMDLASQ | NIHIC_66.10 | | 0.248 |
| | | | | NWND | | | (n = 1) |
| | | | | SRNYTMNLASV | NIHIC_70.2 | | 0.340 |
| | | | | NWND | | | (n = 1) |
| | | | | SHNYTMNLASP | NIHIC_78.6 | | 0.016 |
| | | | | NWND | | | (n = 1) |
| | | | | THNYIMNLASQ | NIHIC_85.1 | | 0.510 |
| | | | | NWND | | | (n = 1) |
| | | | | SRNYTMNLVSQ | Norovirus GII/Hu/JP/2016/ | BAV10402.1 | 0.267 |
| | | | | NWND | GII.P16_GII.4_Sydney2012/ | | (n = 1) |
| | | | | | OC16023 | | |
| VP1 | EWVQYFYQEAA | Class II | 455-470 | EWVQHFYQEAA | NIHIC_70.1 | | 1.073 |
| | PAQS | | | PAQS | | | (n = 1) |
| | | | | EWVSHFYQEAA | NIHIC_78.3 | | 0.735 |
| | | | | PAQS | | | (n = 1) |
| VP1 | DVALLRFVNPD | Class II | 471-485 | DVALLRFVNPE | NIHIC_76.3 | | 0.417 |
| | TGRV | | | TGRV | | | (n = 1) |
| VP1 | DTGRVLFECKL | Class II | 481-495 | ETGRVLFECKL | NIHIC_76.1 | | 0.713 |
| | HKSG | | | HKSG | | | (n = 1) |
| | | | | DTGRVLFECKL | NIHIC_70.1 | | 0.965 |
| | | | | HRSG | | | (n = 1) |
| | | | | DTGRVLFECKL | NIHIC_78.5 | | 0.433 |
| | | | | HKLG | | | (n = 1) |
| | | | | DTGRVLFECKL | NIHIC_78.6 | | 0.265 |
| | | | | HKTG | | | (n = 1) |

Cross-reactivity index = variant response/original response. Variation sequences are indicated with underline. NIHIC = strains from clinical isolates at NIH.

### SUMMARY

Chronic norovirus infection in immunocompromised patients is associated with severe morbidity and potential risk of death due to malnutrition and end-organ damage. Currently, there is no effective treatment. The present study showed that it is possible to generate NST products from healthy seropositive donors using a rapid ex vivo expansion, GMP-applicable, and reproducible platform approach. Cell yields were moderate (median 4.2-fold increase), but they should allow generation of >10⁸ NSTs from as little as 25 million PBMCs, which would permit multiple doses on the order of 10⁷ NSTs/m². Norovirus-specific T cells were polyclonal with both CD4⁺ and CD8⁺ populations, and they showed reactivity to multiple norovirus antigens with a polyfunctional cytokine profile. Moreover, a hierarchy of immunodominance was defined and multiple novel CD4- and CD8-restricted epitopes within NS6 and VP1 antigens were identified. This profile is comparable to the expansion of adenovirus-specific T cells, which tend to have a CD4⁺ T-cell predominance [34]. Use of third-party VSTs with HLA class II-restricted adenovirus hexon protein responses has been successful across several studies [20, 21, 35], suggesting that CD4 T cells may be beneficial in targeting gastrointestinal viruses.

It was also demonstrated that the cross-reactivity of NSTs against variant viral epitopes, which may be indicative of broad clinical application against different circulating viral strains. This is the first report describing the manufacture and comprehensive epitope mapping of NSTs as a proof-of-principle demonstration for the development of a novel off-the-shelf T-cell immunotherapeutic targeting norovirus.

Identification of immunogenic viral antigens is essential for developing immunotherapies. To date, comprehensive T-cell epitope mapping data are lacking in human norovirus although a small number of epitopes have been described [15,16,25]. In the current study, NSTs were generated by stimulating with pooled pepmixes spanning entire norovirus antigens and analyzing the breadth of epitope specificity. Among the 8 antigens, NS6 and VP1, which encode the viral protease and major capsid protein respectively, were recognized by most of the donors and induced the highest magnitude of the responses. The NS6 and VP1 immunogenic epitopes were found to be predominantly HLA class II-restricted. The predominance of CD4⁺ reactive T-cells might be compatible with the notion, supported by an animal study, that murine norovirus reduces the expression of MEW class I proteins and impairs CD8⁺ T-cell recognition and activation [26]. Gerdemann et al. demonstrated similar results in manufacturing human herpesvirus 6-specific T-cells, which might also be explained by herpesvirus-derived immune modulation [27]. Despite the bias of CD4⁺-reactive T-cells, three immunodominant CD8⁺-restricted epitopes were identified, which produced IFN-γ with response to single peptide stimulation.

The strategy used here to identify CD4- and CD8-restricted immunologic epitopes was based on the previous successful epitope mapping approach evaluated with EBV-specific T cells [28]. One potential problem with this method is that not all epitopes may be detected using ex vivo expanded T cells. In a recent study, Malm et al identified human norovirus HLA-A*02:01-restricted minimal 10 mer epitope TMFPHIIVDV (TMF) in VP1 antigen by screening unexpanded PBMCs with matrix peptide approach [16]. NSTs generated from 4 HLA-A*02:01+ donors were evaluated, but the TMF epitope was not identified. One possible explanation for this difference would be antigenic competition for binding to HLA molecules might lead to under-detection of TMF epitope in expanding T-cells with multiple peptide libraries. However, the GMP-compliant overlapping peptide-based method described here allowed the identification of multiple target epitopes, which could minimize the likelihood of viral immune escape and may have superior clinical efficacy compared to targeting a single antigen in isolation [29].

Norovirus, similar to other RNA viruses, is known to evolve quickly and undergo rapid immune escape. An analysis of the norovirus variants present in the stool of immunocompromised patients with chronic norovirus infection showed a diverse norovirus population [30]. The newly identified epitope sequences disclosed herein were compared with virus sequences from the patients with chronic norovirus infection and a high degree of cross-reactivity was observed in NSTs targeting NS6 and VP1, including both CD4- and CD8-restricted epitopes. Cross-reactive T-cell responses have been shown to play a role in protection among viruses with high degree of sequence identity such as dengue virus and Zika virus [31]. This is also similar to previous observations with adenovirus-specific T cells that were reported to be cross-reactive to different adenovirus serogroups [32]. Furthermore, Muftuoglu et al recently showed the advantage of shared epitopes among the members of the Polyomaviridae family by administering BK virus-specific T-cells to treat patients with PML [23]. They found that patients had reduced clinical symptoms and clearance of JC virus after the infusion of T-cell products. Thus, the cross-reactivity of NST responses to variant epitopes has promising implications for adoptive immunotherapy for patients with chronic norovirus infection.

In summary, functionally active NSTs can be generated from seropositive healthy donors in 10 days. These products have wide norovirus antigen recognition and show polyfunctionality, polyclonality, and cross-reactivity to variant epitopes. Furthermore, the identification of multiple novel CD4- and CD8-restricted epitopes may help to identify protective immunogens for a norovirus vaccine as well as a rationale for the creation of third-party banks of NST products. Therefore, it is envisioned that this strategy could translate to future clinical studies to evaluate the safety and efficacy of vaccine approaches and adoptively transferred third-party, partially HLA-matched NST products.

### REFERENCES

1. Glass R I, Parashar U D, Estes M K. Norovirus Gastroenteritis. N Engl J Med 2009; 361:1776-1785.
2. Robilotti E, Deresinski S, Pinsky B A. Norovirus. Clin Microbiol Rev 2015; 28:134-164.
3. Bok K, Green K Y. Norovirus Gastroenteritis in Immunocompromised Patients. N Engl J Med 2012; 367:2126-2132.
4. Green K Y. Norovirus infection in immunocompromised hosts. Clin. Microbiol. Infect. 2014; 20:717-723.
5. Bok K, Prevots D R, Binder A M, et al. Epidemiology of Norovirus Infection Among Immunocompromised Patients at a Tertiary Care Research Hospital, 2010-2013. Open Forum Infect Dis 2016; 3:ofw169.
6. Frange P, Touzot F, Debré M, et al. Prevalence and clinical impact of norovirus fecal shedding in children with inherited immune deficiencies. J Infect Dis 2012; 206:1269-1274.
7. Woodward J, Gkrania-Klotsas E, Kumararatne D. Chronic norovirus infection and common variable immunodeficiency. Clin. Exp. Immunol. 2017; 188:363-370.
8. Angarone M, Ison M G. Diarrhea in solid organ transplant recipients. Curr. Opin. Infect. Dis. 2015; 28:308-316.
9. Roos-Weil D, Ambert-Balay K, Lanternier F, et al. Impact of norovirus/sapovirus-related diarrhea in renal transplant recipients hospitalized for diarrhea. Transplantation 2011; 92:61-69.
10. Schwartz S, Vergoulidou M, Schreier E, et al. Norovirus gastroenteritis causes severe and lethal complications after chemotherapy and hematopoietic stem cell transplantation. Blood 2011; 117:5850-5856.
11. Chachu K A, Strong D W, LoBue A D, Wobus C E, Baric R S, Virgin H W. Antibody Is Critical for the Clearance of Murine Norovirus Infection. J Virol 2008; 82:6610-6617.
12. Chachu K A, LoBue A D, Strong D W, Baric R S, Virgin H W. Immune mechanisms responsible for vaccination against and clearance of mucosal and lymphatic norovirus infection. PLoS Pathog 2008; 4.
13. Wingfield T, Gallimore C I, Xerry J, et al. Chronic norovirus infection in an HIV-positive patient with persistent diarrhoea: A novel cause. J Clin Virol 2010; 49:219-222.
14. De Ravin S S, Wu X, Moir S, et al. Lentiviral hematopoietic stem cell gene therapy for X-linked severe combined immunodeficiency. Sci Transl Med 2016; 8.
15. Malm M, Tamminen K, Vesikari T, Blazevic V. Norovirus-specific memory T cell responses in adult human donors. Front Microbiol 2016; 7.
16. Malm M, Vesikari T, Blazevic V. Identification of a first human norovirus CD8+ T cell epitope restricted to HLA-A*0201 allele. Front Immunol 2018; 9.
17. Leen A M, Myers G D, Sili U, et al. Monoculture-derived T lymphocytes specific for multiple viruses expand and produce clinically relevant effects in immunocompromised individuals. Nat Med 2006; 12:1160-1166.
18. Gerdemann U, Katari U L, Papadopoulou A, et al. Safety and clinical efficacy of rapidly-generated trivirus-directed T cells as treatment for adenovirus, EBV, and CMV infections after allogeneic hematopoietic stem cell transplant. Mol Ther 2013; 21:2113-2121.
19. Papadopoulou A, Gerdemann U, Katari U L, et al. Activity of broad-spectrum T cells as treatment for AdV, EBV, CMV, BKV, and HHV6 infections after HSCT. Sci Transl Med 2014; 6.
20. Leen A M, Bollard C M, Mendizabal A M, et al. Multicenter study of banked third-party virus-specific T cells to treat severe viral infections after hematopoietic stem cell transplantation. Blood 2013; 121:5113-5123.
21. Tzannou I, Papadopoulou A, Naik S, et al. Off-the-shelf virus-specific T cells to treat B K virus, human herpesvirus 6, cytomegalovirus, Epstein-Barr virus, and adenovirus infections after allogeneic hematopoietic stem-cell transplantation. J Clin Oncol 2017; 35:3547-3557.
22. Neuenhahn M, Albrecht J, Odendahl M, et al. Transfer of minimally manipulated CMV-specific T cells from stem cell or third-party donors to treat CMV infection after allo-HSCT. Leukemia 2017; 31:2161-2171.
23. Muftuoglu M, Olson A, Marin D, et al. Allogeneic B K Virus—Specific T Cells for Progressive Multifocal Leukoencephalopathy. N Engl J Med 2018; 379:1443-1451.
24. Karangwa C K, Parra G I, Bok K, Johnson J A, Levenson E A, Green K Y. Sequential Gastroenteritis Outbreaks in a Single Year Caused by Norovirus Genotypes GII.2 and GII.6 in an Institutional Setting. Open forum Infect Dis 2017; 4:ofx236.
25. LoBue A D, Lindesmith L C, Baric R S. Identification of Cross-Reactive Norovirus CD4+ T Cell Epitopes. J Virol 2010; 84:8530-8538.
26. Fritzlar S, Jegaskanda S, Aktepe T E, et al. Mouse Norovirus Infection Reduces the Surface Expression of Major Histocompatibility Complex Class I Proteins and Inhibits CD8+ T Cell Recognition and Activation. J Virol 2018; 92.
27. Gerdemann U, Keukens L, Keirnan J M, et al. Immunotherapeutic strategies to prevent and treat human herpesvirus 6 reactivation after allogeneic stem cell transplantation. Blood 2013; 121:207-18.
28. Straathof K C, Leen A M, Buza E L, et al. Characterization of Latent Membrane Protein 2 Specificity in CTL Lines from Patients with EBV-Positive Nasopharyngeal Carcinoma and Lymphoma. J Immunol 2005; 175:4137-4147.
29. Goldszmid R S, Dzutsev A, Trinchieri G. Host immune response to infection and cancer: Unexpected commonalities. Cell Host Microbe 2014; 15:295-305.
30. Bull R A, Eden J-S, Luciani F, McElroy K, Rawlinson W D, White P A. Contribution of intra- and interhost dynamics to norovirus evolution. J Virol 2012; 86:3219-29.
31. Wen J, Tang W W, Sheets N, et al. Identification of Zika virus epitopes reveals immunodominant and protective roles for dengue virus cross-reactive CD8+ T cells. Nat Microbiol 2017; 2:17036.
32. Leen A M, Sili U, Vanin E F, et al. Conserved CTL epitopes on the adenovirus hexon protein expand subgroup cross-reactive and subgroup-specific CD8+ T cells. Blood 2004; 104:2432-40.
33. Camargo J F, Wieder E D, et al. Deep functional immunophenotyping predicts risk of cytomegalovirus reactivation after hematopoietic cell transplantation. Blood 2019, 133:867-877.
34. Leen A M, Christin A, Khalil M, et al. Identification of hexon-specific CD4 and CD8 T-cell epitopes for vaccine and immunotherapy. J Virol 2008; 82:546-54.
35. Withers B, Blyth E, Clancy L E, et al. Long-term control of recurrent or refractory viral infections after allogeneic HSCT with third-party virus-specific T cells. Blood Adv 2017; 1:2193-205.

1. An isolated T-cell composition comprising one or a plurality of T-cell subpopulations, wherein each T-cell subpopulation is specific for one or a plurality of norovirus antigens.

2. The composition of claim 1, wherein the one or plurality of norovirus antigens are chosen from one or a combination of: NS1-2, NS3, NS4, NS5, NS6, NS7, VP1, or VP2, or functional fragments thereof.

3. The composition of claim 1 or 2, wherein each of the one or plurality of T-cell subpopulations are primed and expanded separately from each other.

4. The composition of any of claims 1 through 3, wherein each of the one or plurality of T-cell subpopulations are primed and expanded ex vivo.

5. The composition of any of claims 1 through 4, wherein each of the T-cell subpopulations is primed and expanded using a group of peptides comprising peptides specific to each norovirus antigen that are HLA-restricted to one or more HLA alleles of a donor cell source.

6. The composition of any of claims 1 through 5, wherein each of the T-cell subpopulations is combined in the T-cell composition in a defined ratio, wherein the defined ratio is based on either total cell number or normalized cell activity.

7. The composition of any of claims 1 through 6, wherein each of the T-cell subpopulation is specific for a norovirus antigen.

8. The composition of any of claims 1 through 7, wherein the T-cell composition consists of from about one to about five T-cell subpopulations.

9. The composition of any of claims 1 through 8, wherein the composition comprises at least about 45% of a first T-cell subpopulation, at least about 10% of a second T-cell subpopulation, and at least about 5% of a third T-cell subpopulation, and at least about 5% of a fourth T-cell subpopulation.

10. The composition of any of claims 1 to 9, wherein one or more of the T-cell subpopulations is derived from umbilical cord blood.

11. The composition of any of claims 1 to 10, wherein each of the T-cell subpopulations is primed and expanded using a group of peptides comprising peptides specific to each norovirus antigen that are HLA-restricted to at least a donor's HLA-A alleles, HLA-B alleles, and HLA-DR alleles.

12. The composition of any of claims 1 to 11, wherein each of the T-cell subpopulations is primed and expanded using a group of peptides comprising peptides specific to each norovirus antigen that are HLA-restricted to at least one of the donor's HLA-A alleles, at least one of the donor's HLA-B allele, and at least one of the donor's HLA-DR alleles.

13. The composition of any of claims 1 to 12, wherein each of the T-cell subpopulations is primed and expanded using a group of peptides comprising peptides specific to each norovirus antigen that are HLA-restricted to at least both of the donor's HLA-A alleles, at least both of the donor's HLA-B allele, and at least both of the donor's HLA-DR alleles.

14. The composition of any of claims 1 to 13, wherein the HLA-A alleles are selected from a group comprising HLA-A*01, HLA-A*02:01, HLA-A*03, HLA-A*11:01, HLA-A*24:02, HLA-A*26, and HLA-A*68:01.

15. The composition of any of claims 1 to 14, wherein the HLA-B alleles are selected from a group comprising HLA-B*07:02, HLA-B*08, HLA-B*15:01 (B62), HLA-B*18, HLA-B*27:05, HLA-B*35:01, and HLA-B*58:02.

16. The composition of any of claims 1 to 15, wherein the HLA-DR alleles are selected from a group comprising HLA-DRB1*0101, HLA-DRB1*0301 (DR17), HLA-DRB1*0401 (DR4Dw4), HLA-DRB1*0701, HLA-DRB1*1101, and HLA-DRB1*1501 (DR2b).

17. A pharmaceutical composition comprising: (i) any one or plurality of compositions of any of claims 1 to 16; and (ii) a pharmaceutically acceptable carrier.

18. A method of treating norovirus infection in a subject comprising administering an effective amount of the composition of any of claims 1 to 17 to a subject in need thereof.

19. The method of claim 18, wherein the norovirus infection is acute.

20. The method of claim 18, wherein the norovirus infection is a chronic infection.

21. The method of any of claims 18 to 20, wherein the T-cell composition has at least one HLA allele or HLA allele combination in common with the subject.

22. The method of any of claims 18 to 20, wherein the T-cell composition has more than one HLA alleles or HLA allele combinations in common with the subject.

23. The method of any of claims 18 to 20, wherein the administration comprises administering a first dose followed by at least one additional dose, wherein the additional dose is administered at an interval selected from every 1 week, every 2 weeks, every 3 weeks, every 4 weeks, every 5 weeks, every 6 weeks, every 7 weeks or every 8 weeks.

24. The method of any of claims 18 to 23, wherein the therapeutically effective amount of cells results in >1 log-fold reduction of detectable virus as measured by ELISpot or detection of norovirus genome levels.

25. A method of treating a subject with a norovirus infection comprising:
i) determining an HLA subtype of the subject;
ii) identifying two or more norovirus antigens associated with tissue from the subject for targeting with norovirus antigen-specific T-cell subpopulations;
iii) selecting a stored T-cell subpopulation having the highest activity against each targeted norovirus antigen through one or more HLA-alleles shared between the subject and the T-cell subpopulations, wherein each T-cell subpopulation is specific for one or a plurality of norovirus antigens;
iv) combining each selected stored T-cell subpopulation to create a T-cell composition; and,
v) administering an effective amount of the T-cell composition to the subject.

26. The method of claim 25, wherein each of the T-cell subpopulations are primed and expanded separately from each other ex vivo.

27. A method of treating a patient with a norovirus infection comprising:
i) determining the HLA subtype of the patient;
ii) determining the norovirus specific antigen expression profile of the patient's norovirus;
iii) identifying two or more norovirus specific antigens expressed by the patient's norovirus for targeting with norovirus antigen-specific T-cell subpopulations;
iv) selecting one banked T-cell subpopulation having the highest activity against each targeted norovirus specific antigen through one or more HLA-alleles shared between the patient and the T-cell subpopulations, wherein each T-cell subpopulation is specific for a single norovirus specific antigen, wherein each T-cell subpopulation is specific for a different norovirus specific antigen, wherein each of the T-cell subpopulations are primed and expanded separately from each other, wherein each of the T-cell subpopulations are expanded ex vivo;
v) combining each selected banked T-cell subpopulation to create a T-cell composition; and,
vi) administering an effective amount of the T-cell composition to the patient.

28. A method of treating a patient with a norovirus infection comprising:
i) determining the HLA subtype of the patient;
ii) determining the norovirus specific antigen expression profile of the patient's norovirus;
iii) identifying two or more norovirus specific antigens expressed by the patient's norovirus for targeting with norovirus antigen-specific T-cell subpopulations;
iv) selecting one banked T-cell subpopulation having the highest activity against each targeted norovirus specific antigen through one or more HLA-alleles shared between the patient and the T-cell subpopulations, wherein each T-cell subpopulation is specific for a single norovirus specific antigen, wherein each of the T-cell subpopulations is specific for a different norovirus specific antigen, wherein each of the T-cell subpopulations are primed and expanded separately from each other, wherein each of the T-cell subpopulations are primed and expanded ex vivo;
v) combining each selected banked T-cell subpopulation to create a first T-cell composition;
vi) administering an effective amount of the first T-cell composition to the patient;
vii) monitoring the patient's response to the first T-cell composition by measuring the presence of circulating norovirus antigen specific T-cells
viii) monitoring changes to the patient's norovirus specific antigen expression profile;
ix) if the patient's norovirus specific antigen expression profile has changed, identifying two or more norovirus specific antigens expressed by the patient's norovirus for targeting with norovirus antigen-specific T-cell subpopulations, wherein if the patient is showing a robust response to any specific norovirus antigen T-cell subpopulation(s) from the first T-cell composition, exclude targeting that norovirus specific antigen;
x) selecting one banked T-cell subpopulation having the highest activity against each targeted norovirus specific antigen from step ix) through one or more HLA-alleles shared between the patient and the T-cell subpopulations, wherein each T-cell subpopulation is specific for a single norovirus specific antigen, wherein each of the T-cell subpopulations is specific for a different norovirus specific antigen, wherein each of the T-cell subpopulations are primed and expanded separately from each other, wherein each of the T-cell subpopulations are primed and expanded ex vivo;
xi) combining each selected banked T-cell subpopulation to create a second T-cell composition;
xii) administering an effective amount of the second T-cell composition to the patient; and,
xiii) optionally repeating steps viii) to xii); and
xiv) combining each selected banked T-cell subpopulation to create a third T-cell composition; and
xv) administering an effective amount of the third T-cell composition to the patient.

29. A method of treating a patient with a norovirus infection comprising:
i) generating one or more norovirus specific T-cell subpopulations from the patient or a healthy relative of the patient, wherein each T-cell subpopulation is specific for a single norovirus specific antigen, wherein each T-cell subpopulation is specific for a different norovirus specific antigen, wherein each of the T-cell subpopulations are primed and expanded separately from each other, wherein each of the T-cell subpopulations are expanded ex vivo;
ii) combining each T-cell subpopulation to create a T-cell composition; and,
iii) administering an effective amount of the T-cell composition to the patient.

30. A library of isolated T-cell subpopulations comprising two or more characterized T-cell subpopulations,
wherein each T-cell subpopulation has been derived from an allogeneic donor;
wherein each T-cell subpopulation is specific for one or plurality of norovirus antigens;
wherein each of the T-cell subpopulations are primed and expanded separately from each other;
wherein each of the T-cell subpopulations are primed and expanded ex vivo;
wherein each of the T-cell subpopulation has been characterized by:
i) HLA-phenotype;
ii) specificity to its specific norovirus antigen;
iii) epitope or epitopes each T-cell subpopulation is specific to;
iv) which MHC Class I and Class II the T-cell subpopulation is restricted to;
v) antigenic activity through the T-cell's corresponding HLA-allele; and
vi) immune effector subtype concentration;
wherein the characterization of each T-cell subpopulation is recorded in a database for future reference, and the T-cell subpopulations are cryopreserved for future use.

31. The methods of any of claims 18 to 26, wherein one or more T-cell subpopulations is primed and expanded with an overlapping peptide library.

32. A composition comprising two or more isolated T-cell subpopulations,
wherein each T-cell subpopulation is specific for a single norovirus antigen;
wherein each of the T-cell subpopulations are primed and expanded separately from each other ex vivo;
wherein each of the T-cell subpopulations are combined in the T-cell composition in a defined ratio, wherein the defined ratio is based on either total cell number or normalized cell activity; and
wherein the single norovirus antigen comprises one or a combination of antigens chosen from: VP1 and NS6, or a functional fragment thereof.


SEQUENCE LISTING (146 sequences)

SEQ ID NO: 1 - protein, 1699 aa, Norovirus
MKMASNDASAAAVANSNNDIAKSSSDGVLSNMAVTFKRALGARPKQPPPKEIPPRPPRPPTPELVKKIPPPPPNGEDELVVSYSAKDGVSGLPELTTVRQPEETNTAFSVPPLNQRESRDAKEPLTGTIIEMWDGEIYHYGLYVERGLILGVHKPPAAISLAKVELAPLSLFWRPVYTPQYLISPDTLRRLHGESFPYTAFDNNCYAFCCWVLDLNDSWLSRRMIQRTTGFFRPYQDWNRKPLPTMDDSKLKKVANIFLCTLSSLFTRPIKDIIGKLKPLNILNILATCDWTFAGIVESLILLAELFGVFWTPPDVSAMIAPLLGDYELQGPEDLAVELVPIVMGGIGLVLGFTKEKIGKMLSSAASTLRACKDLGAYGLEILKLVMKWFFPKKEEANELAMVRSIEDAVLDLEAIENNHMTTLLKDKDSLATYMRTLDLEEEKARKLSTKSASPDIVGTINSLLARIAAARSLVHRAKEELSSRPRPVVVMISGKPGIGKTHLARELAKKIAASLTGDQRVGLIPRNGVDHWDAYKGERVVLWDDYGMSNPIHDALRLQELADTCPLTLNCDRIENKGKVFDSDAIIITTNLANPAPLDYVNFEACSRRIDFLVYAEAPEVEKAKRDFPGQPDMWKNAFSPDFSHIKLSLAPQGGFDKNGNTPHGKGVMKTLTTGSLIARASGLLHERLDEYELQGPALTTFNFDRNKILAFRQLAAENKYGLMDTMRVGKQLKDVKTMSDLKQALKNIAIKKCQIVYNGSTYTLEADGKGSVKVDKVQSAIVQTNNELAGALHHLRCARIRYYVKCVQEALYSIIQIAGAAFVTTRIAKRMNIQNLWSKPQVEDTEEMANKDGCLKPKDDEEFVVSSDDIKTEGKKGKNKSGRGKKHTAFSSKGLSDEEYDEYKRIREERNGKYSIEEYLQDRDRYYEEVAIARATEEDFCEEEEAKIRQRIFRPTRKQRKEERASLGLVTGSEIRKRNPEDFKPKGKLWADDDRSVDYNEKLNFEAPPSIWSRIVNFGSGWGFWVSPSLFITSTHVIPQSAKEFFGVPIKQIQIHKSGEFCRLRFPKPIRTDVTGMILEEGAPEGTVATLLIKRPTGELMPLAARMGTHATMKIQGRTVGGQMGMLLTGSNAKSMDLGTTPGDCGCPYIYKRGNDYVVIGVHTAAARGGNTVICATQGSEGEATLEGGDSKGTYCGAPILGPGSAPKLSTKTKFWRSSTTPLPPGTYEPAYLGGKDPRVKGGPSLQQVMRDQLKPFTEPRGKPPRPNVLEAAKKTIINVLEQTIDPPQKWSFAQACASLDKTTSSGHPHHMRKNDCWNGESFTGKLADQASKANLMFEEGKSMTPVYTGALKDELVKTDKVYGKVKKRLLWGSDLATMIRCARAFGGLMDELKAHCVTLPVRVGMNMNEDGPIIFEKHSRYRYHYDADYSRWDSTQQRDVLAAALEIMVKFSPEPHLAQIVAEDLLSPSVMDVGDFQISISEGLPSGVPCTSQWNSIAHWLLTLCALSEVTDLSPDIIQANSLFSFYGDDEIVSTDIKLDPEKLTAKLKEYGLKPTRPDKTEGPLVISEDLDGLTFLRRTVTRDPAGWFGKLEQSSILRQMYWTRGPNHEDPFETMIPHSQRPIQLMSLLGEAALHGPAFYSKISKLVIAELKEGGMDFYVPRQEPMFRWMRFSDLSTWEGDRNLAPSFVNEDGVE

SEQ ID NO: 2 - protein, 358 aa, Norovirus
RESRDAKEPLTGTIIEMWDGEIYHYGLYVERGLILGVHKPPAAISLAKVELAPLSLFWRPVYTPQYLISPDTLRRLHGESFPYTAFDNNCYAFCCWVLDLNDSWLSRRMIQRTTGFFRPYQDWNRKPLPTMDDSKLKKVANIFLCTLSSLFTRPIKDIIGKLKPLNILNILATCDWTFAGIVESLILLAELFGVFWTPPDVSAMIAPLLGDYELQGPEDLAVELVPIVMGGIGLVLGFTKEKIGKMLSSAASTLRACKDLGAYGLEILKLVMKWFFPKKEEANELAMVRSIEDAVLDLEAIENNHMTTLLKDKDSLATYMRTLDLEEEKARKLSTKSASPDIVGTINSLLARIAAARS

SEQ ID NO: 3 - protein, 102 aa, Norovirus
VMISGKPGIGKTHLARELAKKIAASLTGDQRVGLIPRNGVDHWDAYKGERVVLWDDYGMSNPIHDALRLQELADTCPLTLNCDRIENKGKVFDSDAIIITTN

SEQ ID NO: 4 - protein, 368 aa, Norovirus
SPDFSHIKLSLAPQGGFDKNGNTPHGKGVMKTLTTGSLIARASGLLHERLDEYELQGPALTTFNFDRNKILAFRQLAAENKYGLMDTMRVGKQLKDVKTMSDLKQALKNIAIKKCQIVYNGSTYTLEADGKGSVKVDKVQSAIVQTNNELAGALHHLRCARIRYYVKCVQEALYSIIQIAGAAFVTTRIAKRMNIQNLWSKPQVEDTEEMANKDGCLKPKDDEEFVVSSDDIKTEGKKGKNKSGRGKKHTAFSSKGLSDEEYDEYKRIREERNGKYSIEEYLQDRDRYYEEVAIARATEEDFCEEEEAKIRQRIFRPTRKQRKEERASLGLVTGSEIRKRNPEDFKPKGKLWADDDRSVDYNEKLNFE

SEQ ID NO: 5 - protein, 181 aa, Norovirus
APPSIWSRIVNFGSGWGFWVSPSLFITSTHVIPQSAKEFFGVPIKQIQIHKSGEFCRLRFPKPIRTDVTGMILEEGAPEGTVATLLIKRPTGELMPLAARMGTHATMKIQGRTVGGQMGMLLTGSNAKSMDLGTTPGDCGCPYIYKRGNDYVVIGVHTAAARGGNTVICATQGSEGEATLE

SEQ ID NO: 6 - protein, 467 aa, Norovirus
PKLSTKTKFWRSSTTPLPPGTYEPAYLGGKDPRVKGGPSLQQVMRDQLKPFTEPRGKPPRPNVLEAAKKTIINVLEQTIDPPQKWSFAQACASLDKTTSSGHPHHMRKNDCWNGESFTGKLADQASKANLMFEEGKSMTPVYTGALKDELVKTDKVYGKVKKRLLWGSDLATMIRCARAFGGLMDELKAHCVTLPVRVGMNMNEDGPIIFEKHSRYRYHYDADYSRWDSTQQRDVLAAALEIMVKFSPEPHLAQIVAEDLLSPSVMDVGDFQISISEGLPSGVPCTSQWNSIAHWLLTLCALSEVTDLSPDIIQANSLFSFYGDDEIVSTDIKLDPEKLTAKLKEYGLKPTRPDKTEGPLVISEDLDGLTFLRRTVTRDPAGWFGKLEQSSILRQMYWTRGPNHEDPFETMIPHSQRPIQLMSLLGEAALHGPAFYSKISKLVIAELKEGGMDFYVPRQEPMFRWMR

SEQ ID NO: 7 - protein, 540 aa, Norovirus
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKVIFAAVPPNFPTEGLSPSQVTMFPHIVVDVRQLEPVLIPLPDVRNNFYHYNQSNDPTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSRNYTMNLASQNWNDYDPTEEIPAPLGTPDFVGKIQGVLTQTTRTDGSTRGHKATVYTGSADFAPKLGRVQFETDTDRDFEANQNTKFTPVGVIQDGGTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAV

SEQ ID NO: 8 - protein, 268 aa, Norovirus
MAGAFFAGLASDVLGSGLGSLINAGAGAINQKVEFENNRKLQQASFQFSSNLQQASFQHDKEMLQAQIEATKKLQQEMMKVKQAMLLEGGFFETDAARGAINAPMTKALDWSGTRYWAPDARTTTYNAGRFSTPQPSGALPGRANLRDAVPARGSSSKSSNSSTATSVYSNQTTSTRLGSTAVSGTSVSSFPSTARTRSWVEDQSRNLSPSMRGAHNISFVTPPSSRSSSQGTVSTVPKEVLDSWTGAFNTRRQPLFAHIRKRGESRA

SEQ ID NO: 9 - protein, 312 aa, Homo sapiens
RMSIGLLCCAALSLLWAGPVNAGVTGTPKFGVLKTGGSMTLGCAGDMNHEYMSWYRGDPGMGLRLIHYSVGAGITDGGEVPNGYNVSRSTTEDFPLRLLSAAPSGTSVYFCASSYVGNTGELFFGEGSRLTVLEDLKNVFPPEVAVFEPSEAEISHTCKATLVCLATGFYPDHVELSWWVNGKEVHSGVSTDPGPLKEGPALNDSRYCLSSRLRVSATFWGNPRNHFRCGVGFYGLSENDEWTQDRAKPVTQIVSAEAWGRADCGFTSESYQQGVLSATILYEILLGKATLYAVLVSALVLMAMVKRKDSRG

SEQ ID NO: 10 - protein, 274 aa, Homo sapiens
METLLGLLILWLGLGWVSSKGEVTGIPAALSVPEGENLVLNCSFTDSAIYNLGWFRGDPGKGLTSLLLIGSSGREGTSGRLNASLDKSSGRSTLYIAASGPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFCNLSVIGFRILLLKVAGFNLLMTLRLWSS

SEQ ID NO: 11 - protein, 15 aa, Artificial Sequence
  Norovirus peptide
PSYSGRNTHNVHLAP

SEQ ID NO: 12 - protein, 15 aa, Artificial Sequence
  Norovirus peptide
GSGWGFWVSPSLFIT

SEQ ID NO: 13 - protein, 15 aa, Artificial Sequence
  Norovirus peptide
FWVSPSLFITSTHVI

SEQ ID NO: 14 - protein, 15 aa, Artificial Sequence
  Norovirus peptide
SLFITSTHVIPQSAK

SEQ ID NO: 15 - protein, 15 aa, Artificial Sequence
  Norovirus peptide
PQSAKEFFGVPIKQI

SEQ ID NO: 16 - protein, 15 aa, Artificial Sequence
  Norovirus peptide
EFFGVPIKQIQIHKS

SEQ ID NO: 17 - protein, 15 aa, Artificial Sequence
  Norovirus peptide
PIKQIQIHKSGEFCR

SEQ ID NO: 18 - protein, 15 aa, Artificial Sequence
  Norovirus peptide
LRFPKPIRTDVTGMI

SEQ ID NO: 19 - protein, 15 aa, Artificial Sequence
  Norovirus peptide
PIRTDVTGMILEEGA

SEQ ID NO: 20 - protein, 15 aa, Artificial Sequence
  Norovirus peptide
LEEGAPEGTVATLLI

SEQ ID NO: 21 - protein, 15 aa, Artificial Sequence
  Norovirus peptide
PEGTVATLLIKRPTG

SEQ ID NO: 22 - protein, 15 aa, Artificial Sequence
  Norovirus peptide
GNDYVVIGVHTAAAR

SEQ ID NO: 23 - protein, 15 aa, Artificial Sequence
  Norovirus peptide
VIGVHTAAARGGNTV

SEQ ID NO: 24 - protein, 15 aa, Artificial Sequence
  Norovirus peptide
PVVGAAIAAPVAGQQ

SEQ ID NO: 25 - protein, 15 aa, Artificial Sequence
  Norovirus peptide
LGPDLNPYLSHLARM

SEQ ID NO: 26 - protein, 15 aa, Artificial Sequence
  Norovirus peptide
VRNNFYHYNQSNDPT

SEQ ID NO: 27 - protein, 15 aa, Artificial Sequence
  Norovirus peptide
IKLIAMLYTPLRANN

SEQ ID NO: 28 - protein, 15 aa, Artificial Sequence
  Norovirus peptide
MLYTPLRANNAGDDV

SEQ ID NO: 29 - protein, 15 aa, Artificial Sequence
  Norovirus peptide
AGDDVFTVSCRVLTR

SEQ ID NO: 30 - protein, 15 aa, Artificial Sequence
  Norovirus peptide
PSPDFDFIFLVPPTV

SEQ ID NO: 31 - protein, 15 aa, Artificial Sequence
  Norovirus peptide
DFIFLVPPTVESRTK

SEQ ID NO: 32 - protein, 15 aa, Artificial Sequence
  Norovirus peptide
EMTNSRFPIPLEKLF

SEQ ID NO: 33 - protein, 15 aa, Artificial Sequence
  Norovirus peptide
RFPIPLEKLFTGPSS

SEQ ID NO: 34 - protein, 15 aa, Artificial Sequence
  Norovirus peptide
SRNYTMNLASQNWND

SEQ ID NO: 35 - protein, 15 aa, Artificial Sequence
  Norovirus peptide
EWVQYFYQEAAPAQS

SEQ ID NO: 36 - protein, 15 aa, Artificial Sequence
  Norovirus peptide
APAQSDVALLRFVNP

SEQ ID NO: 37 - protein, 15 aa, Artificial Sequence
  Norovirus peptide
DVALLRFVNPDTGRV

SEQ ID NO: 38 - protein, 15 aa, Artificial Sequence
  Norovirus peptide
DTGRVLFECKLHKSG

SEQ ID NO: 39 - protein, 15 aa, Artificial Sequence
  Norovirus peptide
VNQFYTLAPMGNGTG

SEQ ID NO: 40 - protein, 9 aa, Artificial Sequence
  Norovirus peptide
YVVIGVHTA

SEQ ID NO: 41 - protein, 9 aa, Artificial Sequence
  Norovirus peptide variant
FVVIGVHTA

SEQ ID NO: 42 - protein, 9 aa, Artificial Sequence
  Norovirus peptide variant
LVVIGVHTA

SEQ ID NO: 43 - protein, 15 aa, Artificial Sequence
  Norovirus peptide
GSGWGFWVSPSLFIT

SEQ ID NO: 44 - protein, 15 aa, Artificial Sequence
  Norovirus peptide variant
GTGWGFWVSPSLFIT

SEQ ID NO: 45 - protein, 15 aa, Artificial Sequence
  Norovirus peptide
PQSAKEFFGVPIKQI

SEQ ID NO: 46 - protein, 15 aa, Artificial Sequence
  Norovirus peptide variant
PQGAKEFFGVPIKQI

SEQ ID NO: 47 - protein, 15 aa, Artificial Sequence
  Norovirus peptide variant
PQGAQEFFGVPIKQI

SEQ ID NO: 48 - protein, 15 aa, Artificial Sequence
  Norovirus peptide variant
PQGAKEFFGVSIKQI

SEQ ID NO: 49 - protein, 15 aa, Artificial Sequence
  Norovirus peptide variant
PQGAKEFFGVPIKQT

SEQ ID NO: 50 - protein, 15 aa, Artificial Sequence
  Norovirus peptide variant
PQGAQEFFGVSIKQI

SEQ ID NO: 51 - protein, 15 aa, Artificial Sequence
  Norovirus peptide variant
PQGTQEFFGVSIKQI

SEQ ID NO: 52 - protein, 15 aa, Artificial Sequence
  Norovirus peptide variant
PQGSQEFFGVSIKQI

SEQ ID NO: 53 - protein, 15 aa, Artificial Sequence
  Norovirus peptide variant
PAGITEAFGVPIKQI

SEQ ID NO: 54 - protein, 15 aa, Artificial Sequence
  Norovirus peptide variant
PKGITEAFGVPINQI

SEQ ID NO: 55 - protein, 15 aa, Artificial Sequence
  Norovirus peptide variant
PKGITEAFGVPMNQI

SEQ ID NO: 56 - protein, 15 aa, Artificial Sequence
  Norovirus peptide
EFFGVPIKQIQIHKS

SEQ ID NO: 57 - protein, 15 aa, Artificial Sequence
  Norovirus peptide variant
EAFGVPIKQIQIHKS

SEQ ID NO: 58 - protein, 15 aa, Artificial Sequence
  Norovirus peptide variant
EFFGVSIKQIQIHKS

SEQ ID NO: 59 - protein, 15 aa, Artificial Sequence
  Norovirus peptide variant
EFFGVPIKQTQIHKS

SEQ ID NO: 60 - protein, 15 aa, Artificial Sequence
  Norovirus peptide variant
EFFGVPIKQIQVHKS

SEQ ID NO: 61 - protein, 15 aa, Artificial Sequence
  Norovirus peptide variant
EAFGVPINQIQIHKS

SEQ ID NO: 62 - protein, 15 aa, Artificial Sequence
  Norovirus peptide variant
EAFGVPMNQIQIHKS

SEQ ID NO: 63 - protein, 15 aa, Artificial Sequence
  Norovirus peptide
PIKQIQIHKSGEFCR

SEQ ID NO: 64 - protein, 15 aa, Artificial Sequence
  Norovirus peptide variant
SIKQIQIHKSGEFCR

SEQ ID NO: 65 - protein, 15 aa, Artificial Sequence
  Norovirus peptide variant
PINQIQIHKSGEFCR

SEQ ID NO: 66 - protein, 15 aa, Artificial Sequence
  Norovirus peptide variant
PIKQTQIHKSGEFCR

SEQ ID NO: 67 - protein, 15 aa, Artificial Sequence
  Norovirus peptide variant
PIKQIQVHKSGEFCR

SEQ ID NO: 68 - protein, 15 aa, Artificial Sequence
  Norovirus peptide variant
PMNQIQIHKSGEFCR

SEQ ID NO: 69 - protein, 15 aa, Artificial Sequence
  Norovirus peptide
LRFPKPIRTDVTGMI

SEQ ID NO: 70 - protein, 15 aa, Artificial Sequence
  Norovirus peptide variant
LRFPKPIRTDVAGMI

SEQ ID NO: 71 - protein, 15 aa, Artificial Sequence
  Norovirus peptide variant
FRFPKPIRPDVTGMI

SEQ ID NO: 72 - protein, 15 aa, Artificial Sequence
  Norovirus peptide variant
LRFPKPIRPDVSGMI

SEQ ID NO: 73 - protein, 15 aa, Artificial Sequence
  Norovirus peptide
PIRTDVTGMILEEGA

SEQ ID NO: 74 - protein, 15 aa, Artificial Sequence
  Norovirus peptide variant
PIRPDVTGMILEEGA

SEQ ID NO: 75 - protein, 15 aa, Artificial Sequence
  Norovirus peptide variant
PIRTDVAGMILEEGA

SEQ ID NO: 76 - protein, 15 aa, Artificial Sequence
  Norovirus peptide variant
PIRPDVSGMILEEGA

SEQ ID NO: 77 - protein, 15 aa, Artificial Sequence
  Norovirus peptide
LEEGAPEGTVATLLI

SEQ ID NO: 78 - protein, 15 aa, Artificial Sequence
  Norovirus peptide variant
LEEGAPGGTVATLLI

SEQ ID NO: 79 - protein, 15 aa, Artificial Sequence
  Norovirus peptide variant
LEEGAPEGTVVTLLI

SEQ ID NO: 80 - protein, 15 aa, Artificial Sequence
  Norovirus peptide variant
LEEGAPEGTVATVLI

SEQ ID NO: 81 - protein, 15 aa, Artificial Sequence
  Norovirus peptide variant
LEEGAPEGTVASILI

SEQ ID NO: 82 - protein, 15 aa, Artificial Sequence
  Norovirus peptide variant
LEEGAPEGTVVSILI

SEQ ID NO: 83 - protein, 15 aa, Artificial Sequence
  Norovirus peptide
PEGTVATLLIKRPTG

SEQ ID NO: 84 - protein, 15 aa, Artificial Sequence
  Norovirus peptide variant
PEGTVVTLLIKRPTG

SEQ ID NO: 85 - protein, 15 aa, Artificial Sequence
  Norovirus peptide variant
PEGTVATVLIKRPTG

SEQ ID NO: 86 - protein, 15 aa, Artificial Sequence
  Norovirus peptide variant
PEGTVATLLIKRSTG

SEQ ID NO: 87 - protein, 15 aa, Artificial Sequence
  Norovirus peptide variant
PGGTVATLLIKRSTG

SEQ ID NO: 88 - protein, 15 aa, Artificial Sequence
  Norovirus peptide variant
PEGTVVTLLIKRSTG

SEQ ID NO: 89 - protein, 15 aa, Artificial Sequence
  Norovirus peptide variant
PEGTVASILIKRTTG

SEQ ID NO: 90 - protein, 15 aa, Artificial Sequence
  Norovirus peptide variant
PEGTVVSILIKRTTG

SEQ ID NO: 91 - protein, 15 aa, Artificial Sequence
  Norovirus peptide
GNDYVVIGVHTAAAR

SEQ ID NO: 92 - protein, 15 aa, Artificial Sequence
  Norovirus peptide variant
ENDYVVIGVHTAAAR

SEQ ID NO: 93 - protein, 15 aa, Artificial Sequence
  Norovirus peptide variant
GNDFVVIGVHTAAAR

SEQ ID NO: 94 - protein, 15 aa, Artificial Sequence
  Norovirus peptide variant
GNDLVVIGVHTAAAR

SEQ ID NO: 95 - protein, 9 aa, Artificial Sequence
  Norovirus peptide
GRNTHNVHL

SEQ ID NO: 96 - protein, 9 aa, Artificial Sequence
  Norovirus peptide variant
GINTHNVHL

SEQ ID NO: 97 - protein, 9 aa, Artificial Sequence
  Norovirus peptide
FPGEQLLFF

SEQ ID NO: 98 - protein, 9 aa, Artificial Sequence
  Norovirus peptide variant
LPGEQLLFF

SEQ ID NO: 99 - protein, 9 aa, Artificial Sequence
  Norovirus peptide variant
FPGEQILFF

SEQ ID NO: 100 - protein, 15 aa, Artificial Sequence
  Norovirus peptide
PVVGAAIAAPVAGQQ

SEQ ID NO: 101 - protein, 15 aa, Artificial Sequence
  Norovirus peptide variant
PVAGAAIAAPVAGQQ

SEQ ID NO: 102 - protein, 15 aa, Artificial Sequence
  Norovirus peptide
LGPDLNPYLSHLARM

SEQ ID NO: 103 - protein, 15 aa, Artificial Sequence
  Norovirus peptide variant
LGPGLNPYLSHLARM

SEQ ID NO: 104 - protein, 15 aa, Artificial Sequence
  Norovirus peptide variant
LGPDLNPYLSHLSRM

SEQ ID NO: 105 - protein, 15 aa, Artificial Sequence
  Norovirus peptide
VRNNFYHYNQSNDPT

SEQ ID NO: 106 - protein, 15 aa, Artificial Sequence
  Norovirus peptide variant
VRNNFYHYNQSNDST

SEQ ID NO: 107 - protein, 15 aa, Artificial Sequence
  Norovirus peptide variant
VRNNFYHYNQINEPT

SEQ ID NO: 108 - protein, 15 aa, Artificial Sequence
  Norovirus peptide
IKLIAMLYTPLRANN

SEQ ID NO: 109 - protein, 15 aa, Artificial Sequence
  Norovirus peptide variant
LKLIAMLYTPLRANN

SEQ ID NO: 110 - protein, 15 aa, Artificial Sequence
  Norovirus peptide
DFIFLVPPTVESRTK

SEQ ID NO: 111 - protein, 15 aa, Artificial Sequence
  Norovirus peptide variant
DFIFLVPPTIESRTK

SEQ ID NO: 112 - protein, 15 aa, Artificial Sequence
  Norovirus peptide variant
DFIFLVPPTVESKTK

SEQ ID NO: 113 - protein, 15 aa, Artificial Sequence
  Norovirus peptide
RFPIPLEKLFTGPSS

SEQ ID NO: 114 - protein, 15 aa, Artificial Sequence
  Norovirus peptide variant
RFPIPLEKLFTGPSN

SEQ ID NO: 115 - protein, 15 aa, Artificial Sequence
  Norovirus peptide
SRNYTMNLASQNWND

SEQ ID NO: 116 - protein, 15 aa, Artificial Sequence
  Norovirus peptide variant
SHNYTMNLASQNWND

SEQ ID NO: 117 - protein, 15 aa, Artificial Sequence
  Norovirus peptide variant
SRNYSMNLASQNWND

SEQ ID NO: 118 - protein, 15 aa, Artificial Sequence
  Norovirus peptide variant
SRNYSMDLASQNWND

SEQ ID NO: 119 - protein, 15 aa, Artificial Sequence
  Norovirus peptide variant
SRNYTMNLASVNWND

SEQ ID NO: 120 - protein, 15 aa, Artificial Sequence
  Norovirus peptide variant
SHNYTMNLASPNWND

SEQ ID NO: 121 - protein, 15 aa, Artificial Sequence
  Norovirus peptide variant
THNYIMNLASQNWND

SEQ ID NO: 122 - protein, 15 aa, Artificial Sequence
  Norovirus peptide variant
SRNYTMNLVSQNWND

SEQ ID NO: 123 - protein, 15 aa, Artificial Sequence
  Norovirus peptide
EWVQYFYQEAAPAQS

SEQ ID NO: 124 - protein, 15 aa, Artificial Sequence
  Norovirus peptide variant
EWVQHFYQEAAPAQS

SEQ ID NO: 125 - protein, 15 aa, Artificial Sequence
  Norovirus peptide variant
EWVSHFYQEAAPAQS

SEQ ID NO: 126 - protein, 15 aa, Artificial Sequence
  Norovirus peptide
DVALLRFVNPDTGRV

SEQ ID NO: 127 - protein, 15 aa, Artificial Sequence
  Norovirus peptide variant
DVALLRFVNPETGRV

SEQ ID NO: 128 - protein, 15 aa, Artificial Sequence
  Norovirus peptide
DTGRVLFECKLHKSG

SEQ ID NO: 129 - protein, 15 aa, Artificial Sequence
  Norovirus peptide variant
ETGRVLFECKLHKSG

SEQ ID NO: 130 - protein, 15 aa, Artificial Sequence
  Norovirus peptide variant
DTGRVLFECKLHRSG

SEQ ID NO: 131 - protein, 15 aa, Artificial Sequence
  Norovirus peptide variant
DTGRVLFECKLHKLG

SEQ ID NO: 132 - protein, 15 aa, Artificial Sequence
  Norovirus peptide variant
DTGRVLFECKLHKTG

SEQ ID NO: 133 - protein, 9 aa, Artificial Sequence
  Norovirus peptide
PSYSGRNTH

SEQ ID NO: 134 - protein, 9 aa, Artificial Sequence
  Norovirus peptide
SYSGRNTHN

SEQ ID NO: 135 - protein, 9 aa, Artificial Sequence
  Norovirus peptide
YSGRNTHNV

SEQ ID NO: 136 - protein, 9 aa, Artificial Sequence
  Norovirus peptide
SGRNTHNVH

SEQ ID NO: 137 - protein, 9 aa, Artificial Sequence
  Norovirus peptide
GRNTHNVHL

SEQ ID NO: 138 - protein, 9 aa, Artificial Sequence
  Norovirus peptide
RNTHNVHLA

SEQ ID NO: 139 - protein, 9 aa, Artificial Sequence
  Norovirus peptide
NTHNVHLAP

SEQ ID NO: 140 - protein, 9 aa, Artificial Sequence
  Norovirus peptide
GNDYVVIGV

SEQ ID NO: 141 - protein, 9 aa, Artificial Sequence
  Norovirus peptide
NDYVVIGVH

SEQ ID NO: 142 - protein, 9 aa, Artificial Sequence
  Norovirus peptide
DYVVIGVHT

SEQ ID NO: 143 - protein, 9 aa, Artificial Sequence
  Norovirus peptide
YVVIGVHTA

SEQ ID NO: 144 - protein, 9 aa, Artificial Sequence
  Norovirus peptide
VVIGVHTAA

SEQ ID NO: 145 - protein, 9 aa, Artificial Sequence
  Norovirus peptide
VIGVHTAAA

SEQ ID NO: 146 - protein, 9 aa, Artificial Sequence
  Norovirus peptide
IGVHTAAAR